(19) United States
(12) Patent Application Publication
(10) Pub. No.: US 2022/0265803 A1
(43) Pub. Date: Aug. 25, 2022

(54) MULTIVALENT PNEUMOCOCCAL POLYSACCHARIDE-PROTEIN CONJUGATE COMPOSITIONS AND METHODS OF USING THE SAME

(71) Applicants: SANOFI PASTEUR INC., Swiftwater, PA (US); SK BIOSCIENCE CO., LTD., Seongnam-si, Gyeonggi-do (KR)

(72) Inventors: Kyungjun AN, Seoul (KR); Hun KIM, Suwon-si, Gyeonggi-do (KR); Sunghyun KIM, Yongin-si, Gyeonggi-do (KR); Moe KYAW, Swiftwater, PA (US); Jeong-min LEE, Anyang-si, Gyeonggi-do (KR); Jinhwan SHIN, Seoul (KR); Philippe TALAGA, Lyon Cedex 07 (FR)

(21) Appl. No.: 17/630,841

(22) PCT Filed: Jul. 27, 2020
(86) PCT No.: PCT/US2020/043729

§ 371 Date: Jan. 27, 2022

Related U.S. Application Data
(60) Provisional application No. 62/949,164, filed on Dec. 17, 2019.

(30) Foreign Application Priority Data
Jul. 31, 2019 (KR) 10-2019-0093276

(51) Int. Cl. A61K 39/09; A61K 47/42; A61K 33/06

(52) U.S. Cl. CPC A61K 39/092; A61K 47/42; A61K 33/06; A61K 2039/55505

(57) ABSTRACT

Provided are multivalent pneumococcal conjugate compositions comprising 22-27 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae CA serotypes are selected from 1, 3, 4, 5, 6A, 6B, 7F, 8, 9N, 9V, 10 A, 11 A, 12F, 14, 15A, 15B, 15C, 18C, 19A, 19F, 22F, 23 A, 23B, 23F, 24F, 33F, and 35B. Also provided are methods of producing the multivalent pneumococcal conjugate compositions and methods of using the same for prophylaxis against Streptococcus pneumoniae infection or disease in a subject. Also provided are immunogenic compositions comprising at least one polysaccharide-protein conjugate wherein the polysaccharide is a capsular polysaccharide from CN Streptococcus pneumoniae serotype 15A, 15C, 23 A, 23B, 24F, and/or 35B and methods of preparing the same.

### CROSS REFERENCE TO RELATED APPLICATIONS

This application claims the benefit of, and relies on the filing date of, U.S. provisional patent application No. 62/949,164, filed 17 Dec. 2019, and Korean patent application number 10-2019-0093276, filed 31 Jul. 2019, the entire disclosures of which are herein incorporated by reference.

### TECHNICAL FIELD

This application relates generally to multivalent pneumococcal conjugate compositions, vaccines comprising the same and methods of using these compositions and vaccines for prophylaxis of Streptococcus pneumoniae infection or disease in a subject.

### BACKGROUND

Pneumococcus (Streptococcus pneumoniae) is a Gram-positive, lancet-shaped, facultative anaerobic bacteria with over 90 known serotypes. Most S. pneumoniae serotypes have been shown to cause disease (such as pneumoniae, bacteremia, meningitis, and Otitis disease), with the 23 most common serotypes accounting for approximately 90% of invasive disease worldwide. Serotypes are classified based on the serological response of the capsular polysaccharides, the most important virulence factor for pneumococcus. Capsular polysaccharides are T-cell independent antigens that induce antibody production in the absence of T helper cells. T-cell independent antigens generally induce antibodies with low affinity and short-lived immune responses with little to no immunological memory.

Initial pneumococcal vaccines included combinations of capsular polysaccharides from different serotypes. These vaccines can confer immunity against S. pneumoniae in patients with developed or healthy immune systems, however, they were not effective in infants, who lack a developed immune system, and elderly subjects, who often have impaired immune function. To improve the immune response to pneumococcal vaccines, particularly in infants and elderly subjects, who are at higher risk to develop S. pneumoniae infection, capsular polysaccharides were conjugated to suitable carrier proteins to create pneumococcal conjugate vaccines. Conjugation to a suitable carrier protein changes the capsular polysaccharide from a T-cell independent antigen to a T-cell dependent antigen. As such, the immune response against the conjugated capsular polysaccharide involves T helper cells, which help induce a more potent and rapid immune response upon re-exposure to the capsular polysaccharide.

There are at least two approaches to developing pneumococcal glycoconjugate vaccines: the single carrier approach and the mixed carrier approach. The immunogenicity of different capsular polysaccharide conjugates may vary depending on the pneumococcal serotype and carrier protein used. In the single carrier approach, the capsular polysaccharides from different serotypes are conjugated to a single protein carrier. Pfizer's PREVNAR series of vaccines is an example of a single carrier approach where the different capsular polysaccharides are conjugated to the CRM₁₉₇ protein carrier, a non-toxic variant of the diphtheria toxoid having a single amino acid substitution of glutamic acid for glycine. The 7-valent PREVNAR vaccine (PREVNAR) was first approved in 2000 and contains the capsular polysaccharides from the S. pneumoniae serotypes that were most prevalent at the time of approval: 4, 6B, 9V, 14, 18C, 19F and 23F. The 13-valent vaccine, PREVNAR 13, added the serotypes 1, 5, 7F, 3, 6A, and 19A to the CRM₁₉₇ protein carrier. Merck is developing a 15-valent V114 vaccine that includes the 13 serotypes present in PREVNAR 13 plus 22F and 33F conjugated to CRM₁₉₇. See U.S. Pat. No. 8,192,746. Merck also discloses a 21-valent pneumococcal conjugate composition (PCV21) that includes the following 21 S. pneumoniae serotypes conjugated to CRM₁₉₇: 3, 6C, 7F, 8, 9N, 10A, 11A, 12F, 15A, 16F, 17F, 19A, 20A, 22F, 23A, 23B, 24F, 31, 33F, 35B, and at least one of 15B, 15C, or de-O-acetylated 15B. See US2019/0192648.

The second pneumococcal conjugate vaccine approach is the mixed carrier approach. In the mixed carrier approach, instead of using a single protein carrier, two or more protein carriers are used, with capsular polysaccharides from specific serotypes conjugated to a first protein carrier and capsular polysaccharides from different serotypes conjugated to at least a second, different protein carrier. For example, GlaxoSmithKline has developed SYNFLORIX, a 10-valent (serotypes 1, 4, 5, 6B, 7F, 9V, 14, 18C, 19F and 23F), mixed carrier, pneumococcal conjugate vaccine that uses H. influenzae protein D, tetanus toxoid, and diphtheria toxoid as the protein carriers. In SYNFLORIX, serotypes 1, 4, 5, 6B, 7F, 9V, 14, and 23F are conjugated to protein D; serotype 18C is conjugated to tetanus toxoid; and serotype 19F is conjugated to diphtheria toxoid. Vesikari et al., PIDJ, 28(4):S66-76 (2009). More recently, Sanofi Pasteur and SK Biosciences have made 16-valent (serotypes 1, 3, 4, 5, 6A, 6B, 7F, 9V, 12F, 14, 18C, 19A, 19F, 22F, 23F, and 33F), 20-valent (serotypes 1, 3, 4, 5, 6A, 6B, 7F, 8, 9V, 10A, 11A, 12F, 14, 15B, 18C, 19A, 19F, 22F, 23F and 33F) and 21-valent (1, 3, 4, 5, 6A, 6B, 7F, 8, 9N, 9V, 10A, 11A, 12F, 14, 15B, 18C, 19A, 19F, 22F, 23F and 33F), mixed carrier, pneumococcal conjugate vaccines, as disclosed in published international applications WO2018/027123, WO2018/027126, WO2019/152921, and WO2019/152925, each of which is incorporated by reference in its entirety. In these mixed carrier, multivalent pneumococcal conjugate vaccines two serotypes (two of serotypes 1, 3, and 5) or four serotypes (serotypes 15B and 22F and two of serotypes 1, 3, and 5) are conjugated to tetanus toxoid, while the remaining serotypes are conjugated to CRM₁₉₇.

While both single carrier and mixed carrier glyoconjugate vaccines have been used to provide varying levels of protection against the pneumococcal serotypes contained in the vaccines, serotype replacement, or an increase in the prevalence of virulent pneumococcal strains/serotypes that are not contained in the glycoconjugate vaccines, has been observed and remains a concern. Daniels et al., J Pediatr Pharmacol Ther. 2016 January-February; 21(1): 27-35.

### SUMMARY

This application provides new and improved multivalent pneumococcal conjugate compositions and vaccines comprising the same. In one aspect, this application provides a multivalent pneumococcal conjugate composition, comprising 22-27 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae serotypes are selected from 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B. Other Streptococcus pneumoniae serotypes of interest can be added to the multivalent pneumococcal conjugate composition. In certain embodiments, each capsular polysaccharide is conjugated to the same protein carrier. In certain embodiments, called mixed carrier embodiments, more than one protein carrier, for example two different protein carriers, are used. For example, in certain embodiments, certain capsular polysaccharides are conjugated to a first protein carrier and the remaining capsular polysaccharides are attached to a second protein carrier. In certain embodiments, the first and second protein carriers comprise CRM₁₉₇ and tetanus toxoid. In certain embodiments, two of the capsular polysaccharides are conjugated to tetanus toxoid and the remaining capsular polysaccharides are conjugated to CRM₁₉₇. In certain embodiments, the two capsular polysaccharides that are conjugated to tetanus toxoid are selected from the group consisting of serotypes 1, 3, and 5. In certain embodiments, the two capsular polysaccharides that are conjugated to tetanus toxoid are selected from the group consisting of serotypes 1, 3, 5, 15B, and 22F. In certain embodiments, four of the capsular polysaccharides are conjugated to tetanus toxoid and the remaining capsular polysaccharides are conjugated to CRM₁₉₇. In certain embodiments, four capsular polysaccharides that are conjugated to tetanus toxoid, wherein two of the four capsular polysaccharides that are conjugated to tetanus toxoid are selected from the group consisting of serotypes 1, 3 and 5, and the remaining two capsular polysaccharides are serotypes 15B and 22F.

In one aspect, the multivalent pneumococcal conjugate composition, comprises 27 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae serotypes are selected from 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B.

In certain embodiments, the capsular polysaccharides from serotypes 1 and 5 are conjugated to tetanus toxoid, and the capsular polysaccharides from serotypes 3, 4, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B are conjugated to CRM₁₉₇. In another embodiment, the capsular polysaccharides from serotypes 1 and 3 are conjugated to tetanus toxoid, and the capsular polysaccharides from serotypes 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B are conjugated to CRM₁₉₇. In yet another embodiment, the capsular polysaccharides from serotypes 3 and 5 are conjugated to tetanus toxoid, and the capsular polysaccharides from serotypes 1, 4, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B are conjugated to CRM₁₉₇.

In certain embodiments, four of the capsular polysaccharides are conjugated to tetanus toxoid and the remaining capsular polysaccharides are conjugated to CRM₁₉₇, wherein two of the four capsular polysaccharides that are conjugated to tetanus toxoid are selected from the group consisting of serotypes 1, 3 and 5, and the remaining two capsular polysaccharides are serotypes 15B and 22F.

In one embodiment the mixed carrier, multivalent pneumococcal conjugate composition comprises 27 different pneumococcal capsular polysaccharide-protein conjugates, wherein the capsular polysaccharides from serotypes 1, 5, 15B and 22F are conjugated to tetanus toxoid, and the capsular polysaccharides from serotypes 3, 4, 6A, 6B, 7F, 8, 9N, 9V, 10A, 11A, 12F, 14, 15A, 15C, 18C, 19A, 19F, 23A, 23B, 23F, 24F, 33F, and 35B are conjugated to CRM₁₉₇.

In another embodiment, the mixed carrier, multivalent pneumococcal conjugate composition comprises 27 different pneumococcal capsular polysaccharide-protein conjugates, wherein the capsular polysaccharides from serotypes 1, 3, 15B and 22F are conjugated to tetanus toxoid, and the capsular polysaccharides from serotypes 4, 5, 6A, 6B, 7F, 8, 9N, 9V, 10A, 11A, 12F, 14, 15A, 15C, 18C, 19A, 19F, 23A, 23B, 23F, 24F, 33F, and 35B are conjugated to CRM₁₉₇.

In another embodiment, the mixed carrier, multivalent pneumococcal conjugate composition comprises 27 different pneumococcal capsular polysaccharide-protein conjugates, wherein the capsular polysaccharides from serotypes 3, 5, 15B and 22F are conjugated to tetanus toxoid, and the capsular polysaccharides from serotypes 1, 4, 6A, 6B, 7F, 8, 9N, 9V, 10A, 11A, 12F, 14, 15A, 15C, 18C, 19A, 19F, 23A, 23B, 23F, 24F, 33F, and 35B are conjugated to CRM₁₉₇.

In certain embodiments, the multivalent pneumococcal conjugate composition, comprises 26 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae serotypes are selected from 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B.

In certain embodiments, the multivalent pneumococcal conjugate composition, comprises 25 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae serotypes are selected from 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B.

In certain embodiments, the multivalent pneumococcal conjugate composition, comprises 24 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae serotypes are selected from 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B.

In certain embodiments, the multivalent pneumococcal conjugate composition, comprises 23 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae serotypes are selected from 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B.

In certain embodiments, the multivalent pneumococcal conjugate composition, comprises 22 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae serotypes are selected from 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B.

In some embodiments, the multivalent pneumococcal conjugate composition further comprises an adjuvant, such as an aluminum-based adjuvant, including, but not limited to aluminum phosphate, aluminum sulfate, and aluminum hydroxide.

Another aspect is directed to the use of the multivalent pneumococcal conjugate composition as a vaccine.

Yet another aspect is directed to a vaccine comprising the multivalent pneumococcal conjugate composition and a pharmaceutically acceptable excipient.

Yet another aspect is directed to a method for prophylaxis of Streptococcus pneumoniae infection or disease in a subject, such as a human, the method comprising administering a prophylactically effective amount of the multivalent pneumococcal conjugate compositions or a vaccine comprising the same to the subject.

In certain embodiments, the subject is a human who is at least 50 years old and the disease is pneumonia or invasive pneumococcal disease (IPD).

In other embodiments, the subject is a human who is at least 6 weeks old and the disease is pneumonia, invasive pneumococcal disease (IPD), or acute otitis media (AOM). In some embodiments, the human subject is 6 weeks to 5 years of age. In other embodiments, the human subject is 2 to 15 months of age or 6 to 17 years of age.

In certain embodiments, the multivalent pneumococcal conjugate composition or vaccine is administered by intramuscular injection. In certain embodiments, the multivalent pneumococcal conjugate composition or vaccine is administered as part of an immunization series.

Yet another aspect is directed to an immunogenic composition comprising at least one polysaccharide-protein conjugate and methods for preparing the same, wherein the polysaccharide in the at least one polysaccharide-protein conjugate is a capsular polysaccharide from Streptococcus pneumoniae serotype 15A.

Yet another aspect is directed to an immunogenic composition comprising at least one polysaccharide-protein conjugate and methods for preparing the same, wherein the polysaccharide in the at least one polysaccharide-protein conjugate is a capsular polysaccharide from Streptococcus pneumoniae serotype 15C.

Yet another aspect is directed to an immunogenic composition comprising at least one polysaccharide-protein conjugate and methods for preparing the same, wherein the polysaccharide in the at least one polysaccharide-protein conjugate is a capsular polysaccharide from Streptococcus pneumoniae serotype 23A.

Yet another aspect is directed to an immunogenic composition comprising at least one polysaccharide-protein conjugate and methods for preparing the same, wherein the polysaccharide in the at least one polysaccharide-protein conjugate is a capsular polysaccharide from Streptococcus pneumoniae serotype 23B.

Yet another aspect is directed to an immunogenic composition comprising at least one polysaccharide-protein conjugate and methods for preparing the same, wherein the polysaccharide in the at least one polysaccharide-protein conjugate is a capsular polysaccharide from Streptococcus pneumoniae serotype 24F.

Yet another aspect is directed to an immunogenic composition comprising at least one polysaccharide-protein conjugate and methods for preparing the same, wherein the polysaccharide in the at least one polysaccharide-protein conjugate is a capsular polysaccharide from Streptococcus pneumoniae serotype 35B.

The foregoing and other objects, features, and advantages of the pneumococcal conjugate compositions will become more apparent from the following detailed description.

### Definitions

In order for the present disclosure to be more readily understood, certain terms are first defined below. Additional definitions for the following terms and other terms may be set forth through the specification.

As used in this specification and the appended claims, the singular forms “a,” “an,” and “the” include plural references unless the context clearly dictates otherwise. Thus for example, a reference to “a method” includes one or more methods, and/or steps of the type described herein and/or which will become apparent to those persons skilled in the art upon reading this disclosure and so forth.

Administer: As used herein, “administering” a composition to a subject means to give, apply or bring the composition into contact with the subject. Administration can be accomplished by any of a number of routes, such as, for example, topical, oral, subcutaneous, intramuscular, intraperitoneal, intravenous, intrathecal and intradermal.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 140%, 13%, 120%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Conjugate: As used herein, and understood from the proper context, the terms “conjugate(s)” or “glycoconjugate(s)” refer to a Streptococcus pneumoniae polysaccharide conjugated to a carrier protein using any covalent or non-covalent bioconjugation strategy.

Degree of oxidation: As used herein, the term “degree of oxidation” (DO) refers to the number of sugar repeat units per aldehyde group generated when a purified or sized saccharide is activated with an oxidizing agent. The degree of oxidation of a saccharide can be determined using routine methods known to those of ordinary skill in the art.

### Embodiments: As used herein, the terms “in certain embodiments,” “in some

embodiments,” or the like, refer to embodiments of all aspects of the disclosure, unless the context clearly indicates otherwise.

Excipient: As used herein, the term “excipient” refers to a non-therapeutic agent that may be included in a composition, for example to provide or contribute to a desired consistency or stabilizing effect.

Mixed carrier: As used herein, a mixed carrier, pneumococcal conjugate composition refers to a pneumococcal conjugate composition having more than one type of protein carrier.

22-valent pneumococcal conjugate composition: As used herein, the term “22-valent pneumococcal conjugate composition(s)” or “PCV-22” refers to a composition comprising pneumococcal capsular polysaccharide-protein conjugates, wherein the pneumococcal capsular polysaccharide-protein conjugates comprise or consist of 22 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae serotypes are selected from 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B.

23-valent pneumococcal conjugate composition: As used herein, the term “23-valent pneumococcal conjugate composition(s)” or “PCV-23” refers to a composition comprising pneumococcal capsular polysaccharide-protein conjugates, wherein the pneumococcal capsular polysaccharide-protein conjugates comprise or consist of 23 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae serotypes are selected from 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B.

24-valent pneumococcal conjugate composition: As used herein, the term “24-valent pneumococcal conjugate composition(s)” or “PCV-24” refers to a composition comprising pneumococcal capsular polysaccharide-protein conjugates, wherein the pneumococcal capsular polysaccharide-protein conjugates comprise or consist of 24 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae serotypes are selected from 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B.

25-valent pneumococcal conjugate composition: As used herein, the term “25-valent pneumococcal conjugate composition(s)” or “PCV-25” refers to a composition comprising pneumococcal capsular polysaccharide-protein conjugates, wherein the pneumococcal capsular polysaccharide-protein conjugates comprise or consist of 25 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae serotypes are selected from 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B.

26-valent pneumococcal conjugate composition: As used herein, the term “26-valent pneumococcal conjugate composition(s)” or “PCV-26” refers to a composition comprising pneumococcal capsular polysaccharide-protein conjugates, wherein the pneumococcal capsular polysaccharide-protein conjugates comprise or consist of 26 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae serotypes are selected from 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B.

27-valent pneumococcal conjugate composition: As used herein, the term “27-valent pneumococcal conjugate composition(s)” or “PCV-27” refers to a composition comprising pneumococcal capsular polysaccharide-protein conjugates, wherein the pneumococcal capsular polysaccharide-protein conjugates comprise or consist of 27 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae serotypes are 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B.

Molecular weight: Unless specified otherwise, as used herein, the term “molecular weight” of a capsular saccharide or a capsular saccharide-carrier protein conjugate refers to the average molecular weight calculated by size exclusion chromatography (SEC) in combination with multi-angle laser light scattering (MALLS).

Multivalent: As used herein, the term “multivalent” refers to a pneumococcal conjugate composition having pneumococcal capsular polysaccharides from more than one Streptococcus pneumoniae serotype.

Pharmaceutically acceptable excipient: The pharmaceutically acceptable excipients useful in this disclosure are conventional. Remington's Pharmaceutical Sciences, by E. W. Martin, Mack Publishing Co., Easton, Pa., 15^{th} Edition (1975), describes compositions and formulations suitable for pharmaceutical delivery of one or more therapeutic compositions, including vaccines, and additional pharmaceutical agents. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. In general, the nature of the excipient will depend on the particular mode of administration being employed. For instance, parenteral formulations usually comprise injectable fluids that include pharmaceutically and physiologically acceptable fluids such as water, physiological saline, balanced salt solutions, buffers, aqueous dextrose, glycerol or the like as a vehicle. For solid compositions (for example, powder, pill, tablet, or capsule forms), conventional non-toxic solid excipients can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, a surface active agent, preservatives, and pH buffering agents and the like, for example sodium acetate or sorbitan monolaurate.

Prophylactically Effective Amount: As defined herein, the term “a prophylactically effective amount” or “a prophylactically effective dose” refers to the amount or dose required to induce an immune response sufficient to delay onset and/or reduce in frequency and/or severity one or more symptoms caused by an infection with Streptococcus pneumoniae.

Prophylaxis: The term “prophylaxis,” as used herein, refers to avoidance of disease manifestation, a delay of onset, and/or reduction in frequency and/or severity of one or more symptoms of a particular disease, disorder or condition (e.g., infection with Streptococcus pneumoniae). In some embodiments, prophylaxis is assessed on a population basis such that an agent is considered to provide prophylaxis against a particular disease, disorder or condition if a statistically significant decrease in the development, frequency, and/or intensity of one or more symptoms of the disease, disorder or condition is observed in a population susceptible to the disease, disorder, or condition.

Subject: As used herein, the term “subject” means any mammal, including mice, rabbits, and humans. In certain embodiments the subject is an adult, an adolescent or an infant. In some embodiments, terms “individual” or “patient” are used and are intended to be interchangeable with “subject.”

### DETAILED DESCRIPTION

The following description of the disclosed embodiment(s) and Examples is merely exemplary in nature and is in no way intended to limit the invention, its application, or uses.

This application provides new and improved multivalent pneumococcal conjugate compositions and vaccines comprising the same. As shown in the examples, robust antibody responses were obtained against the 27 serotypes in PCV-27, including serotypes that are not covered by existing pneumococcal vaccines, such as serotype 15A, serotype 15C, serotype 23A, serotype 23B, serotype 24F, and serotype 35B.

### Pneumococcal Polysaccharide Serotype 15A

The serotype 15A polysaccharide may be obtained directly from the bacteria by using an isolation procedure known to those of ordinary skill in the art (including, but not limited to, the methods disclosed in US Patent Application Publication No. 2006/0228380). In addition, 15A oligosaccharides can be produced using synthetic protocols.

The serotype 15A Streptococcus pneumoniae strain may be obtained from established culture collections (e.g., the Streptococcal Reference Laboratory of the Centers for Disease Control and Prevention (Atlanta, Ga.)) or clinical specimens.

The bacterial cell is typically grown in a medium, such as a soy-based medium. Following fermentation of the bacterial cell producing Streptococcus pneumoniae serotype 15A capsular polysaccharide, the bacterial cell is lysed to produce a cell lysate. Then, the serotype 15A polysaccharide may be isolated from the cell lysate using purification techniques known in the art, including centrifugation, depth filtration, precipitation, ultrafiltration, treatment with activated carbon, diafiltration and/or column chromatography (including, but not limited to, the methods disclosed in US Patent Application Publication No. 2006/0228380).

The purified serotype 15A polysaccharide is conjugated to a carrier protein to form an immunogenic composition comprising at least one polysaccharide-protein conjugate comprising the serotype 15A polysaccharide and the carrier protein. In one aspect, the 15A polysaccharide-protein conjugate can be made by a method comprising the steps of:

(i) subjecting a purified Streptococcus pneumoniae serotype 15A polysaccharide to an acid hydrolysis reaction and heat or a microfluidizer, and then reacting with an oxidizing agent to produce an activated Streptococcus pneumoniae serotype 15A polysaccharide;

(ii) optionally lyophilizing the activated Streptococcus pneumoniae serotype 15A polysaccharide and a carrier protein;

(iii) suspending the activated Streptococcus pneumoniae serotype 15A polysaccharide and the carrier protein in dimethyl sulfoxide (DMSO);

(iv) reacting the activated Streptococcus pneumoniae serotype 15A polysaccharide and the carrier protein with a reducing agent to produce Streptococcus pneumoniae serotype 15A polysaccharide-carrier protein conjugate; and

(v) capping unreacted aldehydes in the Streptococcus pneumoniae serotype 15A polysaccharide-carrier protein conjugate to prepare an immunogenic conjugate comprising the Streptococcus pneumoniae serotype 15A polysaccharide covalently linked to the carrier protein. Further details about the reagents (e.g., oxidizing agent, reducing agent, carrier protein, etc.) and conditions that can be used in this method are disclosed elsewhere in this application, including in the sections that follow and the Examples.

The activated serotype 15A capsular polysaccharide may be characterized by different parameters including, for example, the molecular weight (MW) and/or degree of oxidation (Do).

In one aspect, an activated Streptococcus pneumoniae serotype 15A polysaccharide has a molecular weight of less than 120 kDa before conjugation, including, for example, an activated serotype 15A capsular polysaccharide having a molecular weight of about 10-120 kDa, 50-120 kDa, 70-120 kDa, 70-80 kDa, 70-118 kDa, 114-118 kDa, or about 116 kDa before conjugation. Any whole number within any of the above ranges is contemplated as an embodiment of the present disclosure.

In one aspect, when the molecular weight of the Streptococcus pneumoniae serotype 15A polysaccharide is less than 120 kDa before conjugation, a polysaccharide-protein conjugate of about 1,000-5,000 kDa can be produced, such as a polysaccharide-protein conjugate of about 1,200-4,000 kDa, 1,200-1,500 kDa, 1,200-3,500 kDa, 1,400-4,000 kDa, about 1,200 kDa, about 1,400 kDa, or about 4,000 kDa. Any whole number within any of the above ranges is contemplated as an embodiment of the present disclosure.

The purified serotype 15A polysaccharide may be characterized by a degree of oxidation following activation with an oxidation agent. In one aspect, the activated serotype 15A polysaccharide may have a degree of oxidation ranging from 1 to 15, such as, 4-10, 4-8, 4-5, 5-8, or about 4.

In one aspect, an activated polysaccharide of Streptococcus pneumoniae serotype 15A having an oxidation level (Do) of about 4 is conjugated with a carrier protein to obtain a serotype 15A capsular polysaccharide-protein conjugate with a content of free polysaccharide (Free PS) of 40% or less, such as 5-40%, 20-40%, 25-40%, 20-35%, 25-35%, or 30-35%.

The polysaccharide may become slightly reduced in size during a normal purification procedure. Additionally, as described in the present disclosure, the polysaccharide may be subjected to sizing before conjugation. The molecular weight range mentioned above refers to that of the purified polysaccharide after the final sizing step (e.g., after purification, hydrolysis and activation) before conjugation.

### Pneumococcal Polysaccharide Serotype 15C

The serotype 15C polysaccharide may be obtained directly from the bacteria by using an isolation procedure known to those of ordinary skill in the art (including, but not limited to, the methods disclosed in US Patent Application Publication No. 2006/0228380). In addition, 15C oligosaccharides can be produced using synthetic protocols.

The serotype 15C Streptococcus pneumoniae strain may be obtained from established culture collections (e.g., the Streptococcal Reference Laboratory of the Centers for Disease Control and Prevention (Atlanta, Ga.)) or clinical specimens. Alternatively, the serotype 15C polysaccharide may be obtained by de-O-acetylation of the serotype 15B polysaccharide, typically by alkaline treatment.

The bacterial cell is typically grown in a medium, such as a soy-based medium. Following fermentation of the bacterial cell producing Streptococcus pneumoniae serotype 15C capsular polysaccharide, the bacterial cell is lysed to produce a cell lysate. Then, the serotype 15C polysaccharide may be isolated from the cell lysate using purification techniques known in the art, including centrifugation, depth filtration, precipitation, ultrafiltration, treatment with activated carbon, diafiltration and/or column chromatography (including, but not limited to, the methods disclosed in US Patent Application Publication No. 2006/0228380).

The purified serotype 15C polysaccharide is conjugated to a carrier protein to form an immunogenic composition comprising at least one polysaccharide-protein conjugate comprising the serotype 15C polysaccharide and the carrier protein. In one aspect, the 15C polysaccharide-protein conjugate can be made by a method comprising the steps of:

(i) reacting a purified Streptococcus pneumoniae serotype 15C polysaccharide with an oxidizing agent to produce an activated Streptococcus pneumoniae serotype 15C polysaccharide;

(ii) optionally lyophilizing the activated Streptococcus pneumoniae serotype 15C polysaccharide and a carrier protein;

(iii) suspending the activated Streptococcus pneumoniae serotype 15C polysaccharide and the carrier protein in dimethyl sulfoxide (DMSO) or phosphate buffer;

(iv) reacting the mixture of the activated serotype 15C polysaccharide and the carrier protein with a reducing agent to produce a serotype 15C polysaccharide-carrier protein conjugate; and

(v) capping unreacted aldehydes in the serotype 15C polysaccharide-carrier protein conjugate to prepare an immunogenic conjugate comprising the Streptococcus pneumoniae serotype 15C polysaccharide covalently linked to the carrier protein. Further details about the reagents (e.g., oxidizing agent, reducing agent, carrier protein, etc.) and conditions that can be used in this method are disclosed elsewhere in this application, including in the sections that follow and the Examples.

The activated serotype 15C capsular polysaccharide may be characterized by different parameters including, for example, the molecular weight (MW) and/or degree of oxidation (Do).

In one aspect, an activated Streptococcus pneumoniae serotype 15C polysaccharide before conjugation may have a molecular weight of 200-1,000 kDa, such as 400-800 kDa, 500-775 kDa, 470-775 kDa, 500-770 kDa, 520-680 kDa, 510-770 kDa, 510-550 kDa, 670-770 kDa, or similar molecular weight ranges. Any whole number within any of the above ranges is contemplated as an embodiment of the present disclosure.

A 15C polysaccharide-protein conjugate having a molecular weight of about 1,000-10,000 kDa can be produced, such as a 15C polysaccharide-protein conjugate of about 2,000-6,000 kDa, 2,500-5,000 kDa, 6,000-10,000 kDa, or 6,200-9,400 kDa. Any whole number within any of the above ranges is contemplated as an embodiment of the present disclosure.

The purified serotype 15C polysaccharide may be characterized by a degree of oxidation following activation with an oxidation agent. In one aspect, the activated serotype 15C polysaccharide may have a degree of oxidation ranging from 1 to 40. A degree of oxidation of 8-35, 15-35, 8-20, 8-9, 9-20, or 30-35 may be obtained by adding sodium periodate to a Streptococcus pneumoniae serotype 15C polysaccharide.

In one aspect, an activated polysaccharide of Streptococcus pneumoniae serotype 15C having an oxidation level (Do) of 30-35 is conjugated with a carrier protein to obtain a serotype 15C capsular polysaccharide-protein conjugate with a content of free polysaccharide (Free PS) of 40% or less, such as 5-40%, 20-40%, 25-40%, 20-35%, 25-35%, or 30-35%.

The polysaccharide may become slightly reduced in size during a normal purification procedure. Additionally, as described in the present disclosure, the polysaccharide may be subjected to sizing before conjugation. The molecular weight range mentioned above refers to that of the purified polysaccharide after the final sizing step (e.g., after purification, hydrolysis and activation) before conjugation.

### Pneumococcal Polysaccharide Serotype 23A

The serotype 23A polysaccharide may be obtained directly from the bacteria by using an isolation procedure known to those of ordinary skill in the art (including, but not limited to, the methods disclosed in US Patent Application Publication No. 2006/0228380). In addition, 23A oligosaccharides can be produced using synthetic protocols.

The serotype 23A Streptococcus pneumoniae strain may be obtained from established culture collections (e.g., the Streptococcal Reference Laboratory of the Centers for Disease Control and Prevention (Atlanta, Ga.)) or clinical specimens.

The bacterial cell is typically grown in a medium, such as a soy-based medium. Following fermentation of the bacterial cell producing Streptococcus pneumoniae serotype 23A capsular polysaccharide, the bacterial cell is lysed to produce a cell lysate. Then, the serotype 23A polysaccharide may be isolated from the cell lysate using purification techniques known in the art, including centrifugation, depth filtration, precipitation, ultrafiltration, treatment with activated carbon, diafiltration and/or column chromatography (including, but not limited to, the methods disclosed in US Patent Application Publication No. 2006/0228380).

The purified serotype 23A polysaccharide is conjugated to a carrier protein to form an immunogenic composition comprising at least one polysaccharide-protein conjugate comprising the serotype 23A polysaccharide and the carrier protein. In one aspect, the 23A polysaccharide-protein conjugate can be made by a method comprising the steps of:

(i) reacting a purified Streptococcus pneumoniae serotype 23A with an oxidizing agent to produce an activated Streptococcus pneumoniae serotype 23A polysaccharide;

(ii) optionally lyophilizing the activated Streptococcus pneumoniae serotype 23A polysaccharide and a carrier protein;

(iii) suspending the activated Streptococcus pneumoniae serotype 23A polysaccharide and the carrier protein in dimethyl sulfoxide (DMSO) or phosphate buffer;

(iv) reacting the mixture of the activated Streptococcus pneumoniae serotype 23A polysaccharide and the carrier protein with a reducing agent to produce a Streptococcus pneumoniae serotype 23A polysaccharide-carrier protein conjugate; and

(v) capping unreacted aldehydes in the Streptococcus pneumoniae serotype 23A polysaccharide-carrier protein conjugate to prepare an immunogenic conjugate comprising the Streptococcus pneumoniae serotype 23A polysaccharide covalently linked to the carrier protein. Further details about the reagents (e.g., oxidizing agent, reducing agent, carrier protein, etc.) and conditions that can be used in this method are disclosed elsewhere in this application, including in the sections that follow and the Examples.

The activated serotype 23A capsular polysaccharide may be characterized by different parameters including, for example, the molecular weight (MW) and/or degree of oxidation (Do).

In one aspect, an activated Streptococcus pneumoniae serotype 23A polysaccharide before conjugation may have a molecular weight of 300-700 kDa, such as 400-650 kDa, 430-650 kDa, 470-650 kDa, 470-570 kDa, 470-490 kDa, or similar molecular weight ranges. Any whole number within any of the above ranges is contemplated as an embodiment of the present disclosure.

A serotype 23A polysaccharide-protein conjugate of about 2,000-7,000 kDa can be produced using the methods disclosed herein. The molecular weight of the serotype 23A capsular polysaccharide-protein conjugate may be about 2,000-4,000 kDa, 4,000-7,000 kDa, 4,200-6,700 kDa, 4,350-6,650 kDa, 5,000-6,700 kDa, about 4,300 kDa, about 5,000 kDa, or about 6,600 kDa. Any whole number within any of the above ranges is contemplated as an embodiment of the present disclosure.

The purified serotype 23A polysaccharide may be characterized by a degree of oxidation following activation with an oxidation agent. In one aspect, the activated serotype 23A polysaccharide may have a degree of oxidation ranging from 4 to 25, such as 6-24, 6-18, 9-18, 6-9, 6-10, 6-11, or 9-11.

In one aspect, an activated polysaccharide of Streptococcus pneumoniae serotype 23A having an oxidation level (Do) of 9-11 is conjugated with a carrier protein to obtain a serotype 23A capsular polysaccharide-protein conjugate with a content of free polysaccharide (Free PS) of 40% or less, such as 5-40%, 20-40%, 25-40%, 20-35%, 25-35%, or 30-35%.

Any suitable buffer can be used for conjugation, including DMSO or phosphate buffer. When DMSO is used, the reaction concentration of the polysaccharide can be 2.5 mg/mL or less, including, for example 1.0 mg/mL to 2.5 mg/mL, 1.0 mg/mL to 2.0 mg/mL, or 1.0 mg/mL to 1.5 mg/mL. When phosphate buffer is used the reaction concentration of the polysaccharide can be 10 to 20 mg/mL, including, for example, 15 mg/mL.

The polysaccharide may become slightly reduced in size during a normal purification procedure. Additionally, as described in the present disclosure, the polysaccharide may be subjected to sizing before conjugation.

### Pneumococcal Polysaccharide Serotype 23B

The serotype 23B polysaccharide may be obtained directly from the bacteria by using an isolation procedure known to those of ordinary skill in the art (including, but not limited to, the methods disclosed in US Patent Application Publication No. 2006/0228380). In addition, 23B oligosaccharides can be produced using synthetic protocols.

The serotype 23B Streptococcus pneumoniae strain may be obtained from established culture collections (e.g., the Streptococcal Reference Laboratory of the Centers for Disease Control and Prevention (Atlanta, Ga.)) or clinical specimens.

The bacterial cell is typically grown in a medium, such as a soy-based medium. Following fermentation of the bacterial cell producing Streptococcus pneumoniae serotype 23B capsular polysaccharide, the bacterial cell is lysed to produce a cell lysate. Then, the serotype 23B polysaccharide may be isolated from the cell lysate using purification techniques known in the art, including centrifugation, depth filtration, precipitation, ultrafiltration, treatment with activated carbon, diafiltration and/or column chromatography (including, but not limited to, the methods disclosed in US Patent Application Publication No. 2006/0228380).

The purified serotype 23B polysaccharide is conjugated to a carrier protein to form an immunogenic composition comprising at least one polysaccharide-protein conjugate comprising the serotype 23B polysaccharide and the carrier protein. In one aspect, the 23B polysaccharide-protein conjugate can be made by a method comprising the steps of:

(i) reacting a purified Streptococcus pneumoniae serotype 23B with an oxidizing agent to produce an activated Streptococcus pneumoniae serotype 23B polysaccharide;

(ii) optionally lyophilizing the activated Streptococcus pneumoniae serotype 23B polysaccharide and a carrier protein;

(iii) suspending the activated Streptococcus pneumoniae serotype 23B polysaccharide and the carrier protein in dimethyl sulfoxide (DMSO);

(iv) reacting the mixture of the activated Streptococcus pneumoniae serotype 23B polysaccharide and the carrier protein with a reducing agent to produce a Streptococcus pneumoniae serotype 23B polysaccharide-carrier protein conjugate; and

(v) capping unreacted aldehydes in the Streptococcus pneumoniae serotype 23B polysaccharide-carrier protein conjugate to prepare an immunogenic conjugate comprising the Streptococcus pneumoniae serotype 23B polysaccharide covalently linked to the carrier protein. Further details about the reagents (e.g., oxidizing agent, reducing agent, carrier protein, etc.) and conditions that can be used in this method are disclosed elsewhere in this application, including in the sections that follow and the Examples.

The activated serotype 23B capsular polysaccharide may be characterized by different parameters including, for example, the molecular weight (MW) and/or degree of oxidation (Do).

In one aspect, an activated Streptococcus pneumoniae serotype 23B polysaccharide before conjugation may have a molecular weight of 100-800 kDa, such as 200-700 kDa, 200-650 kDa, 300-650 kDa, 380-640 kDa, 550-675 kDa, 200-250 kDa, 220-230 kDa, 220-225 kDa, or similar molecular weight ranges. Any whole number within any of the above ranges is contemplated as an embodiment of the present disclosure.

A serotype 23B polysaccharide-protein conjugate of about 2,000-7,000 kDa can be produced using the methods disclosed herein. The molecular weight of the serotype 23B capsular polysaccharide-protein conjugate can range from about 2,000-4,000 kDa, 2,000-5,000, 4,000-7,000 kDa, 2,400-6,800 kDa, 4,600-6,800 kDa, or 6,400-6,800 kDa. Any whole number within any of the above ranges is contemplated as an embodiment of the present disclosure.

The purified serotype 23B polysaccharide may be characterized by a degree of oxidation following activation with an oxidation agent. In one aspect, the activated serotype 23B polysaccharide may have a degree of oxidation of 5.4 or less, such as a degree of oxidation of 1-5.4, 2-5.4, 2.3-5.4, 2-3, or 2.3-2.8.

In one aspect, an activated polysaccharide of Streptococcus pneumoniae serotype 23B having an oxidation level (Do) of 3 or less (as discussed above) is conjugated with a carrier protein to obtain a serotype 23B capsular polysaccharide-protein conjugate with a content of free polysaccharide (Free PS) of 40% or less, such as 5-40%, 20-40%, 25-40%, 20-35%, 25-35%, or 30-35%.

The polysaccharide may become slightly reduced in size during a normal purification procedure. Additionally, as described in the present disclosure, the polysaccharide may be subjected to sizing before conjugation.

### Pneumococcal Polysaccharide Serotype 24F

The serotype 24F polysaccharide may be obtained directly from the bacteria by using an isolation procedure known to those of ordinary skill in the art (including, but not limited to, the methods disclosed in US Patent Application Publication No. 2006/0228380). In addition, 24F oligosaccharides can be produced using synthetic protocols.

The serotype 24F Streptococcus pneumoniae strain may be obtained from established culture collections (e.g., the Streptococcal Reference Laboratory of the Centers for Disease Control and Prevention (Atlanta, Ga.)) or clinical specimens.

The bacterial cell is typically grown in a medium, such as a soy-based medium. Following fermentation of the bacterial cell producing Streptococcus pneumoniae serotype 24F capsular polysaccharide, the bacterial cell is lysed to produce a cell lysate. Then, the serotype 24F polysaccharide may be isolated from the cell lysate using purification techniques known in the art, including centrifugation, depth filtration, precipitation, ultrafiltration, treatment with activated carbon, diafiltration and/or column chromatography (including, but not limited to, the methods disclosed in US Patent Application Publication No. 2006/0228380).

The purified serotype 24F polysaccharide is conjugated to a carrier protein to form an immunogenic composition comprising at least one polysaccharide-protein conjugate comprising the serotype 24F polysaccharide and the carrier protein. In one aspect, the 24F polysaccharide-protein conjugate can be made by a method comprising the steps of:

(i) subjecting a purified Streptococcus pneumoniae serotype 24F polysaccharide to an acid hydrolysis reaction or a microfluidizer, and then reacting with an oxidizing agent to produce an activated Streptococcus pneumoniae serotype 24F polysaccharide;

(ii) optionally lyophilizing the activated Streptococcus pneumoniae serotype 24F polysaccharide and a carrier protein;

(iii) suspending the activated Streptococcus pneumoniae serotype 24F polysaccharide and the carrier protein in dimethyl sulfoxide (DMSO) or phosphate buffer;

(iv) reacting the activated Streptococcus pneumoniae serotype 24F polysaccharide and the carrier protein with a reducing agent to produce Streptococcus pneumoniae serotype 24F polysaccharide-carrier protein conjugate; and

(v) capping unreacted aldehydes in the Streptococcus pneumoniae serotype 24F polysaccharide-carrier protein conjugate to prepare an immunogenic conjugate comprising the Streptococcus pneumoniae serotype 24F polysaccharide covalently linked to the carrier protein. Further details about the reagents (e.g., oxidizing agent, reducing agent, carrier protein, etc.) and conditions that can be used in this method are disclosed elsewhere in this application, including in the sections that follow and the Examples.

The activated serotype 24F capsular polysaccharide may be characterized by different parameters including, for example, the molecular weight (MW) and/or degree of oxidation (Do).

In one aspect, an activated Streptococcus pneumoniae serotype 24F polysaccharide before conjugation may have a molecular weight of 100-500 kDa, such as 150-350 kDa, 200-400 kDa, 200-300 kDa, 225-275 kDa, 240-260 kDa, 245-255 kDa, about 250 kDa, or similar molecular weight ranges. Any whole number within any of the above ranges is contemplated as an embodiment of the present disclosure.

A serotype 24F polysaccharide-protein conjugate of about 1,000-5,000 kDa can be produced using the methods disclosed herein. The molecular weight of the serotype 24F capsular polysaccharide-protein conjugate can range from about 1,500-5,000 kDa, 2,000-4,500, or 2,500-3,500 kDa. Any whole number within any of the above ranges is contemplated as an embodiment of the present disclosure.

The purified serotype 24F polysaccharide may be characterized by a degree of oxidation following activation with an oxidation agent. In one aspect, the activated serotype 24F polysaccharide may have a degree of oxidation of at least 90, including about 90-100.

In one aspect, a molar equivalent of reducing agent of 2.0 or less can be used in a step of reacting an activated serotype 24F polysaccharide having a degree of oxidation of at least 90 and a carrier protein to obtain a serotype 24F capsular polysaccharide-protein conjugate with a free sugar (Free PS) of 40% or less, such as 5-40%, 20-40%, 25-40%, 20-35%, 25-35%, or 30-35%. A molar equivalent of reducing agent of 0.5 to 1.2, 1.0 to 1.2 or about 1.2 can be used.

The polysaccharide may become slightly reduced in size during a normal purification procedure. Additionally, as described in the present disclosure, the polysaccharide may be subjected to sizing before conjugation.

### Pneumococcal Polysaccharide Serotype 35B

The serotype 35B polysaccharide may be obtained directly from the bacteria by using an isolation procedure known to those of ordinary skill in the art (including, but not limited to, the methods disclosed in US Patent Application Publication No. 2006/0228380). In addition, 35B oligosaccharides can be produced using synthetic protocols.

The serotype 35B Streptococcus pneumoniae strain may be obtained from established culture collections (e.g., the Streptococcal Reference Laboratory of the Centers for Disease Control and Prevention (Atlanta, Ga.)) or clinical specimens.

The bacterial cell is typically grown in a medium, such as a soy-based medium. Following fermentation of the bacterial cell producing Streptococcus pneumoniae serotype 35B capsular polysaccharide, the bacterial cell is lysed to produce a cell lysate. Then, the serotype 35B polysaccharide may be isolated from the cell lysate using purification techniques known in the art, including centrifugation, depth filtration, precipitation, ultrafiltration, treatment with activated carbon, diafiltration and/or column chromatography (including, but not limited to, the methods disclosed in US Patent Application Publication No. 2006/0228380).

The purified serotype 35B polysaccharide is conjugated to a carrier protein to form an immunogenic composition comprising at least one polysaccharide-protein conjugate comprising the serotype 35B polysaccharide and the carrier protein. In one aspect, the 35B polysaccharide-protein conjugate can be made by a method comprising the steps of:

(i) reacting a purified Streptococcus pneumoniae serotype 35B with an oxidizing agent to produce an activated Streptococcus pneumoniae serotype 35B polysaccharide;

(ii) optionally lyophilizing the activated Streptococcus pneumoniae serotype 35B polysaccharide and a carrier protein;

(iii) suspending the activated Streptococcus pneumoniae serotype 35B polysaccharide and the carrier protein in dimethyl sulfoxide (DMSO) or phosphate buffer;

(iv) reacting the activated Streptococcus pneumoniae serotype 35B polysaccharide and the carrier protein with a reducing agent to produce Streptococcus pneumoniae serotype 35B polysaccharide-carrier protein conjugate; and

(v) capping unreacted aldehydes in the Streptococcus pneumoniae serotype 35B polysaccharide-carrier protein conjugate to prepare an immunogenic conjugate comprising the Streptococcus pneumoniae serotype 35B polysaccharide covalently linked to the carrier protein. Further details about the reagents (e.g., oxidizing agent, reducing agent, carrier protein, etc.) and conditions that can be used in this method are disclosed elsewhere in this application, including in the sections that follow and the Examples.

The activated serotype 35B capsular polysaccharide may be characterized by different parameters including, for example, the molecular weight (MW) and/or degree of oxidation (Do).

For example, the size of the purified serotype 35B polysaccharide may be reduced, for example by high pressure homogenization or mechanical homogenization, before conjugation to the carrier protein. In one aspect, the activated serotype 35B polysaccharide has a molecular weight of 10 to 20,000 kDa, 10 to 1,000 kDa, 10 to 500 kDa, 10 to 300 kDa, 20 to 200 kDa, or 20 to 120 kDa before conjugation.

The purified serotype 35B polysaccharide may be characterized by a degree of oxidation following activation with an oxidation agent. In one aspect, the activated serotype 35B polysaccharide may have a degree of oxidation of 1-50, 1-45, 1-40, 1-35, 1-30, 2-50, 2-45, 2-40, 2-35, 2-30, 3-40, 3-35, 3-30, 4-40 4-35, or 4-30.

In one aspect, an activated polysaccharide of Streptococcus pneumoniae serotype 35B having an oxidation level (Do) of 4-30 is conjugated with a carrier protein to obtain a serotype 35B capsular polysaccharide-protein conjugate with a content of free polysaccharide (Free PS) of 40% or less, such as 5-40%, 20-40%, 25-40%, 20-35%, 25-35%, or 30-35%.

To produce a serotype 35B glycoconjugate having advantageous immunogenic properties one or more of the following process parameters in the activation (oxidation), conjugation, and/or capping steps can be combined:
in the activation step, a periodate (e.g., sodium or potassium periodate) is reacted with a molar equivalent of 0.005 to 0.5, 0.005 to 0.3, 0.005 to 0.2, or 0.007 to 0.15 per 1M of the serotype 35B polysaccharide;
the activation step may be performed in an aqueous solvent, such as sodium acetate buffer or deionized water;
the activation step may be performed in 0.1 mM to 15 mM or 0.1 to 10 mM sodium acetate buffer;
the activation step may be carried out at pH 4-8 or pH 4-7.5;
in the activation step, the periodate may be treated at 21° C. to 25° C.;
in the activation step, the periodate and serotype 35B polysaccharide may be reacted for 0.5 to 50 hours or 1 to 25 hours;
following the activation step, the activated serotype 35B polysaccharide may be concentrated using, for example, a 30 kDa MWCO ultrafiltration filter;
in the conjugation step, the concentration of the activated serotype 35B polysaccharide in the conjugation reaction may be 5 mg/mL to 30 mg/mL or 10 mg/mL to 20 mg/mL;
in the conjugation step, the initial loading ratio of the carrier protein and activated serotype 35B polysaccharide (PR:PS) may be 1:0.3, 1:0.4, 1:0.5, 1:0.6, 1:0.7, 1:0.8, 1:0.9, 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9 or 1:3, and preferably 1:0.5 to 2;
in the conjugation step, the amount of reducing agent used may be 0.1 to 5 molar or 0.5 to 2 equivalents per 1M activated polysaccharide, preferably 0.5, 0.6, 0.7, 0.8, 0.9, 1, 1.1, 1.2, 1.3, 1.4, 1.5, 1.6, 1.7, 1.8, 1.9 or 2 molar equivalents per 1M activated saccharide, or more preferably 0.8 to 1.6 molar equivalents of reducing agent per 1M activated polysaccharide;
in the conjugation step, the temperature may be 20° C. to 45° C., 30° C. to 40° C., 35 to 40° C., or 37±2° C.;
in the conjugation step, the pH may be 5.5 to 8.5, 5.5 to 7.5, or 6 to 7.5;
in the conjugation step, the carrier protein and activated serotype 35B polysaccharide may be reacted with the reducing agent for 1 to 70 hours or 40 to 60 hours;
following the conjugation step, the yield of serotype 35B glycoconjugate may be at least 20%, 30%, 40%, 50%, 55%, 60%, 65%, 70%, 75%, 80%, 85% or 90%;
in the capping step, sodium borohydride may be treated at 0.5 to 5 molar equivalents per 1M of activated serotype 35B polysaccharide, such as 1 to 3 or 1.5 to 2.5 molar equivalents per 1M of activated serotype 35B polysaccharide, or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3 molar equivalents of sodium borohydride per 1M of activated polysaccharide;
in the capping step, the temperature may be 10 to 40° C., 15 to 30° C., 20 to 26° C., or 23±2° C.;
in the capping step, the reaction time may be 0.5 to 10 hours or 2 to 8 hours; and/or
following the capping step, the serotype 35B glycoconjugate may be concentrated using, for example, a 100 kDa MWCO ultrafiltration filter.

In one exemplary embodiment, the method of producing a serotype 35B glycoconjugate comprises the following steps:

(i) diluting an isolated serotype 35B polysaccharide with sodium acetate buffer (NaOAc, pH 4.5 to pH 6.0) or DW (deionized water);

(ii) reacting the serotype 35B polysaccharide with a 0.005 to 0.5 molar equivalent of sodium periodate to prepare an activated serotype 35B polysaccharide;

(iii) purifying the activated serotype 35B polysaccharide and then mixing with cryoprotectant;

(iv) lyophilizing the activated serotype 35B and carrier protein, respectively;

(v) resuspending the activated serotype 35B polysaccharide and carrier protein in DMSO or phosphate buffer;

(vi) mixing the resuspended activated serotype 35B polysaccharide with the carrier protein and reacting with sodium cyanoborohydride to produce the serotype 35B polysaccharide-carrier protein conjugate;

(vii) capping unreacted aldehyde in serotype 35B polysaccharide-carrier protein conjugate with sodium borohydride; and

(viii) obtaining an immunogenic conjugate comprising Streptococcus pneumoniae serotype 35B polysaccharide covalently linked to the carrier protein.

In another exemplary embodiment, the method of producing a serotype 35B glycoconjugate comprises the following steps:

(i) diluting an isolated serotype 35B polysaccharide with sodium acetate buffer (NaOAc, pH 4.5 to pH 6.0) or DW (deionized water);

(ii) reacting the serotype 35B polysaccharide with a 0.005 to 0.5 molar equivalent of sodium periodate to prepare an activated serotype 35B polysaccharide;

(iii) purifying the activated serotype 35B polysaccharide;

(iv) mixing the activated serotype 35B polysaccharide with a carrier protein, followed by co-lyophilizing;

(v) resuspending co-lyophilized activated serotype 35B polysaccharide and carrier protein in DMSO or phosphate buffer;

(vi) reacting with sodium cyanoborohydride to produce serotype 35B polysaccharide-carrier protein conjugate;

(vii) capping unreacted aldehyde in serotype 35B polysaccharide-carrier protein conjugate with sodium borohydride; and

(viii) obtaining an immunogenic conjugate comprising Streptococcus pneumoniae serotype 35B polysaccharide covalently linked to the carrier protein.

### Pneumococcal Polysaccharide Serotype 22F

The activated serotype 22F capsular polysaccharide may be characterized by different parameters including, for example, the degree of oxidation (Do) following activation with an oxidation agent. In certain embodiments, the activated serotype 22F polysaccharide may have a Do of 20-100, 20-80, 20-60, 20-50, 20-40, 20-35, 25-100, 25-50, 25-35, 28-32, or 29-31. Any whole number within any of the above ranges is contemplated as an embodiment of the present disclosure.

The serotype 22F polysaccharide-protein conjugate may be characterized by different parameters, including, for example, the protein to polysaccharide (PS/PR) ratio, free sugar (Free PS), MSD %, or molecular weight (MALLS) following conjugation. In certain embodiments, the PS/PR ratio of the 22F capsular polysaccharide-protein conjugate (e.g., 22F-TT) may be 0.2 to 1.5, 0.2 to 0.5, 0.3 to 0.4, 0.6 to 1.0, 0.7 to 0.9, or 0.6 to 0.8. In certain embodiments, the 22F capsular polysaccharide-protein conjugate (e.g., 22F-TT) has a Free PS of 40% or less, such as 2-40%, 2-20%, 2-10%, 5-30%, 10-25%, 15-25%, 17-21%, or about 19%. In certain embodiments, the serotype 22F capsular polysaccharide-protein conjugate (e.g., 22F-TT) has an MSD (%) of 5-60%, 5-10%, 5-50%, 10-50%, 25-50%, 40-50%, 42-46% or about 44%. In certain embodiments, the molecular weight of the 22F capsular polysaccharide-protein conjugate (e.g., 22F-TT) can range from about 1,000-6,000 kDa, 2,000-5,000 kDa, 2,500-4,000 kDa, 3,000 to 3,500 kDa, or 3,000 to 3,100 kDa. Any whole number within any of the above ranges is contemplated as an embodiment of the present disclosure.

Any of the above-described parameters for serotype 22F can be combined as desired. For example, in certain embodiments, the activated serotype 22F polysaccharide used to make the serotype 22F polysaccharide-protein conjugate has a Do of about 29-31 with a reaction ratio of protein (TT) to polysaccharide of about 1:1. And in certain embodiments the polysaccharide/carrier protein ratio (PS/PR) in the final conjugate is about 0.6 to 0.8, the Free PS is about 17-21%, and the MSD % is about 42-46%, optionally with a molecular weight of about 3,000 to 3,100 kDa by MALLS.

### Pneumococcal Polysaccharide Serotype 15B

The activated serotype 15B capsular polysaccharide may be characterized by different parameters including, for example, the degree of oxidation (Do) following activation with an oxidation agent. In certain embodiments, the activated serotype 15B polysaccharide may have a degree of oxidation of 1 to 15, 5 to 10, 6 to 8, or about 7. Any whole number within any of the above ranges is contemplated as an embodiment of the present disclosure.

The serotype 15B polysaccharide-protein conjugate may be characterized by different parameters, including, for example, the protein to polysaccharide (PS/PR) ratio, free sugar (Free PS), MSD %, or molecular weight (MALLS) following conjugation. In certain embodiments, the PS/PR ratio of the 15B capsular polysaccharide-protein conjugate (e.g., 15B-TT) may be 0.2 to 1.5, 0.2 to 0.5, 0.3 to 0.4, 0.6 to 1.0, 0.7 to 0.9, or 0.8 to 1.0. In certain embodiments, the 15B capsular polysaccharide-protein conjugate (e.g., 15B-TT) has a Free PS of 30% or less, such as 2-30%, 2-20%, 2-10%, 5-10%, 8-10%, or about 9%. In certain embodiments, the serotype 15B capsular polysaccharide-protein conjugate (e.g., 15B-TT) has an MSD (%) of 50-90%, 60-85%, 65-80%, 70-80%, 74-78%, or about 76%. In certain embodiments, the molecular weight of the 15B capsular polysaccharide-protein conjugate (e.g., 15B-TT) can range from about 2,000-15,000 kDa, 10,000-15,000 kDa, 2,000-10,000 kDa, 3,000 to 7,500 kDa, 4,000 to 6,000 kDa, 5,000 to 6,000 kDa, or 5,500 to 5,600 kDa. Any whole number within any of the above ranges is contemplated as an embodiment of the present disclosure.

Any of the above-described parameters for serotype 15B can be combined as desired. For example, in certain embodiments, the activated serotype 15B polysaccharide used to make the serotype 15B polysaccharide-protein conjugate has a Do of about 7.0 with a reaction ratio of protein (TT) to polysaccharide of about 1.25:1. And in certain embodiments the polysaccharide/carrier protein ratio (PS/PR) in the final conjugate is about 0.8 to 1.0, the Free PS is about 8-10%, and the MSD % is about 74-78%, optionally with a molecular weight of about 5,500 to 5,600 by MALLS.

### Pneumococcal Polysaccharide Serotype 19A

The activated serotype 19A capsular polysaccharide may be characterized by different parameters including, for example, the degree of oxidation (Do) following activation with an oxidation agent. In certain embodiments, the activated serotype 19A polysaccharide may have a degree of oxidation of 20-40, 30-40, 35-40, 30-35, 20-30, 22-28, 24-28, 25-30, or 25-27. Any whole number within any of the above ranges is contemplated as an embodiment of the present disclosure.

The serotype 19A polysaccharide-protein conjugate may be characterized by different parameters, including, for example, the protein to polysaccharide (PS/PR) ratio, free sugar (Free PS), MSD (%) or molecular weight (MALLS) following conjugation. In certain embodiments, the PS/PR ratio of the 19A capsular polysaccharide-protein conjugate (e.g., 19A-CRM₁₉₇) may be 0.2 to 1.5, 0.2 to 0.5, 0.3 to 0.4, 0.6 to 1.0, 0.7 to 0.9, or 0.6 to 0.8. In certain embodiments, the 19A capsular polysaccharide-protein conjugate (e.g., 19A-CRM₁₉₇) has a Free PS of 50% or less, such as 10-40%, 15-40%, 20-40%, 25-40%, 25-35%, 30 to 40%, 30 to 35%, 32 to 34% or about 33%. In certain embodiments, the serotype 19A capsular polysaccharide-protein conjugate (e.g., 19A-CRM₁₉₇) has an MSD (%) of 35-70%, 40-50%, 50-70%, 60-70%, 63-68%, or about 65%. In certain embodiments, the molecular weight of the serotype 19A capsular polysaccharide-protein conjugate (e.g., 19A-CRM₁₉₇) can range from about 2,000-8,000 kDa, 3,500-7,000 kDa, 4,500-6,500 kDa, 5,000 to 6,500 kDa, or 5,250 to 6,250 kDa. Any whole number within any of the above ranges is contemplated as an embodiment of the present disclosure.

Any of the above-described parameters for serotype 19A can be combined as desired. For example, in certain embodiments, the activated serotype 19A polysaccharide used to make the serotype 19A polysaccharide-protein conjugate has a Do of about 25 to 27 with a reaction ratio of protein (CRM₁₉₇) to polysaccharide of about 1:1. And in certain embodiments the polysaccharide/carrier protein ratio (PS/PR) in the final conjugate is about 0.7, the Free PS is about 30-35%, and the MSD % is about 63-68%, optionally with a molecular weight of about 5,250 to 6,250 by MALLS.

### Pneumococcal Polysaccharide Serotype 19F

The activated serotype 19F capsular polysaccharide may be characterized by different parameters including, for example, the degree of oxidation (Do) following activation with an oxidation agent. In certain embodiments, the activated serotype 19F polysaccharide may have a degree of oxidation of 20-50, 30-50, 40-50, 25-35, 20-30, 22-28, 25-30, 23-27, or 24-26. Any whole number within any of the above ranges is contemplated as an embodiment of the present disclosure.

The serotype 19F polysaccharide-protein conjugate may be characterized by different parameters, including, for example, the protein to polysaccharide (PS/PR) ratio, MSD (%) or free sugar (Free PS) following conjugation. In certain embodiments, the PS/PR ratio of the 19F capsular polysaccharide-protein conjugate (e.g., 19F-CRM₁₉₇) may be 0.2 to 1.5, 0.2 to 0.5, 0.3 to 0.4, 0.6 to 1.0, 0.7 to 0.9, or 0.6 to 0.8. In certain embodiments, the serotype 19F capsular polysaccharide-protein conjugate (e.g., 19F-CRM₁₉₇) has an MSD (%) of 25-80%, 35-75%, 40-60%, 70-80%, 75-80%, or about 77%. In certain embodiments, the serotype 19F capsular polysaccharide-protein conjugate (e.g., 19F-CRM₁₉₇) has a Free PS of 30% or less, such as 2-30%, 2-20%, 2-10%, 2-9%, 3-7%, 4-6%, or about 5%.

Any of the above-described parameters for serotype 19F can be combined as desired. For example, in certain embodiments, the activated serotype 19F polysaccharide used to make the serotype 19F polysaccharide-protein conjugate has a Do of about 24 to 26 with a reaction ratio of protein (CRM₁₉₇) to polysaccharide of about 1.5:1. And in certain embodiments the polysaccharide/carrier protein ratio (PS/PR) in the final conjugate is about 0.7, the Free PS is about 4-6%, and the MSD % is about 75-80%.

### Pneumococcal Polysaccharide Serotype 4

The serotype 4 polysaccharide-protein conjugate may be characterized by different parameters, including, for example, the protein to polysaccharide (PS/PR) ratio, free sugar (Free PS), MSD (%), or molecular weight (MALLS) following conjugation. In certain embodiments, the PS/PR ratio of the 4 capsular polysaccharide-protein conjugate (e.g., 4-CRM₁₉₇) may be 0.2 to 1.5, 0.8 to 1.1, 0.8 to 1.3, 0.9 to 1.1, or about 1.0. In certain embodiments, the serotype 4 capsular polysaccharide-protein conjugate (e.g., 4-CRM₁₉₇) has a Free PS of 40% or less, such as 5-30%, 15-35%, 5-15%, 7-13%, 9-11% or about 10%. In certain embodiments, the serotype 4 capsular polysaccharide-protein conjugate (e.g., 4-CRM₁₉₇) has an MSD (%) of 40-80%, 45-75%, 45-55%, 60-75%, or 70-75%. In certain embodiments, the molecular weight of the serotype 4 capsular polysaccharide-protein conjugate (e.g., 4-CRM₁₉₇) can range from about 500-2,500 kDa, 500-1,000 kDa, 1,000-2,000 kDa, 1,500 to 2,000 kDa, 1,800 to 2,000 kDa, or 1,850 to 1,950 kDa. Any whole number within any of the above ranges is contemplated as an embodiment of the present disclosure.

Any of the above-described parameters for serotype 4 can be combined as desired. For example, in certain embodiments, the activated serotype 4 polysaccharide used to make the serotype 4 polysaccharide-protein conjugate has a Do of about 1.4 with a reaction ratio of protein (CRM₁₉₇) to polysaccharide of about 1.25:1. And in certain embodiments the polysaccharide/carrier protein ratio (PS/PR) in the final conjugate is about 1.0, the Free PS is about 9-11%, and the MSD % is about 70-75%, optionally with a molecular weight of about 1,850 to 1,950 by MALLS.

### Pneumococcal Polysaccharide Serotype 9V

The serotype 9V polysaccharide-protein conjugate may be characterized by different parameters, including, for example, the protein to polysaccharide (PS/PR) ratio, free sugar (Free PS), MSD (%) or molecular weight (MALLS) following conjugation. In certain embodiments, the PS/PR ratio of the 9V capsular polysaccharide-protein conjugate (e.g., 9V-CRM₁₉₇) may be 0.2 to 1.5, 0.2 to 0.5, 0.3 to 0.4, 0.8 to 1.3, 1.0 to 1.2, or about 1.1. In certain embodiments, the serotype 9V capsular polysaccharide-protein conjugate (e.g., 9V-CRM₁₉₇) has a Free PS of 35% or less, such as 10-35%, 20-35%, 5-15%, 7-13%, 9-11% or about 10%. In certain embodiments, the serotype 9V capsular polysaccharide-protein conjugate (e.g., 9V-CRM₁₉₇) has an MSD (%) of 40-80%, 45-75%, 45-60%, 50-65%, 55-65, 57-61%, or about 59%. In certain embodiments, the molecular weight of the serotype 9V capsular polysaccharide-protein conjugate (e.g., (9V-CRM₁₉₇) can range from about 500-2,000 kDa, 500-1,500 kDa, 1,000-2,000 kDa, 1,000 to 1,500 kDa, 1,000 to 1,200 kDa, or 1,100 to 1,200 kDa. Any whole number within any of the above ranges is contemplated as an embodiment of the present disclosure.

Any of the above-described parameters for serotype 9V can be combined as desired. For example, in certain embodiments, the activated serotype 9V polysaccharide used to make the serotype 9V polysaccharide-protein conjugate has a Do of about 7.4 with a reaction ratio of protein (CRM₁₉₇) to polysaccharide of about 1.25:1. And in certain embodiments the polysaccharide/carrier protein ratio (PS/PR) in the final conjugate is about 1.1, the Free PS is about 9-11%, and the MSD % is about 57-61%, optionally with a molecular weight of about 1,100 to 1,200 by MALLS.

Multivalent Pneumococcal Conjugate Compositions and Methods of Making the Same

This disclosure provides multivalent pneumococcal conjugate compositions comprising different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae. Different aspects and embodiments of the multivalent pneumococcal conjugate compositions are described herein.

In one aspect, the multivalent pneumococcal conjugate composition comprises pneumococcal capsular polysaccharide-protein conjugates, wherein the pneumococcal capsular polysaccharide-protein conjugates comprise or consist of 22-27 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae serotypes are selected from 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B.

In one aspect, the multivalent pneumococcal conjugate composition comprises pneumococcal capsular polysaccharide-protein conjugates, wherein the pneumococcal capsular polysaccharide-protein conjugates comprise or consist of 27 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae serotypes are 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B. This multivalent pneumococcal conjugate composition is also referred to as PCV-27.

In one aspect, the multivalent pneumococcal conjugate composition comprises pneumococcal capsular polysaccharide-protein conjugates, wherein the pneumococcal capsular polysaccharide-protein conjugates comprise or consist of 26 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae serotypes are selected from 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B. This multivalent pneumococcal conjugate composition is also referred to as PCV-26. In certain embodiments of PCV-26, at least one of the Streptococcus pneumoniae serotypes is 35B. In certain embodiments of PCV-26, the Streptococcus pneumoniae serotypes comprise or consist of 1, 3, 4, 5, 6A, 6B, 7F, 8, 9N, 9V, 10A, 11A, 12F, 14, 15B, 18C, 19A, 19F, 22F, 23F, 33F, and 35B and four serotypes selected from 15A, 15C, 23A, 23B, and 24F. For example, the PCV-26 comprises pneumococcal capsular polysaccharide-protein conjugates, wherein the pneumococcal capsular polysaccharide-protein conjugates may comprise or consist of 26 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae serotypes are:
a) 1, 3, 4, 5, 6A, 6B, 7F, 8, 9N, 9V, 10A, 11A, 12F, 14, 15B, 18C, 19A, 19F, 22F, 23F, 33F, 35B, 15A, 15C, 23A, and 23B;
b) 1, 3, 4, 5, 6A, 6B, 7F, 8, 9N, 9V, 10A, 11A, 12F, 14, 15B, 18C, 19A, 19F, 22F, 23F, 33F, 35B, 15A, 15C, 23A, and 24F;
c) 1, 3, 4, 5, 6A, 6B, 7F, 8, 9N, 9V, 10A, 11A, 12F, 14, 15B, 18C, 19A, 19F, 22F, 23F, 33F, 35B, 15A, 15C, 23B, and 24F.
d) 1, 3, 4, 5, 6A, 6B, 7F, 8, 9N, 9V, 10A, 11A, 12F, 14, 15B, 18C, 19A, 19F, 22F, 23F, 33F, 35B, 15A, 23A, 23B, and 24F; or
e) 1, 3, 4, 5, 6A, 6B, 7F, 8, 9N, 9V, 10A, 11A, 12F, 14, 15B, 18C, 19A, 19F, 22F, 23F, 33F, 35B, 15C, 23A, 23B, and 24F

In one aspect, the multivalent pneumococcal conjugate composition comprises pneumococcal capsular polysaccharide-protein conjugates, wherein the pneumococcal capsular polysaccharide-protein conjugates comprise or consist of 25 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae serotypes are selected from 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B. This multivalent pneumococcal conjugate composition is also referred to as PCV-25. In certain embodiments of PCV-25, at least one of the Streptococcus pneumoniae serotypes is 35B. In certain embodiments of PCV-25, the Streptococcus pneumoniae serotypes comprise or consist of 1, 3, 4, 5, 6A, 6B, 7F, 8, 9N, 9V, 10A, 11A, 12F, 14, 15B, 18C, 19A, 19F, 22F, 23F, 33F, and 35B and three serotypes selected from 15A, 15C, 23A, 23B, and 24F. For example, the PCV-25 comprises pneumococcal capsular polysaccharide-protein conjugates, wherein the pneumococcal capsular polysaccharide-protein conjugates may comprise or consist of 25 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae serotypes are:
a) 1, 3, 4, 5, 6A, 6B, 7F, 8, 9N, 9V, 10A, 11A, 12F, 14, 15B, 18C, 19A, 19F, 22F, 23F, 33F, 35B, 15A, 15C, and 23A;
b) 1, 3, 4, 5, 6A, 6B, 7F, 8, 9N, 9V, 10A, 11A, 12F, 14, 15B, 18C, 19A, 19F, 22F, 23F, 33F, 35B, 15A, 15C, and 23B;
c) 1, 3, 4, 5, 6A, 6B, 7F, 8, 9N, 9V, 10A, 11A, 12F, 14, 15B, 18C, 19A, 19F, 22F, 23F, 33F, 35B, 15A, 15C, and 24F;
d) 1, 3, 4, 5, 6A, 6B, 7F, 8, 9N, 9V, 10A, 11A, 12F, 14, 15B, 18C, 19A, 19F, 22F, 23F, 33F, 35B, 15A, 23A, and 23B;
e) 1, 3, 4, 5, 6A, 6B, 7F, 8, 9N, 9V, 10A, 11A, 12F, 14, 15B, 18C, 19A, 19F, 22F, 23F, 33F, 35B, 15A, 23A, and 24F;
f) 1, 3, 4, 5, 6A, 6B, 7F, 8, 9N, 9V, 10A, 11A, 12F, 14, 15B, 18C, 19A, 19F, 22F, 23F, 33F, 35B, 15A, 23B, and 24F;
g) 1, 3, 4, 5, 6A, 6B, 7F, 8, 9N, 9V, 10A, 11A, 12F, 14, 15B, 18C, 19A, 19F, 22F, 23F, 33F, 35B, 15C, 23A, and 23B;
h) 1, 3, 4, 5, 6A, 6B, 7F, 8, 9N, 9V, 10A, 11A, 12F, 14, 15B, 18C, 19A, 19F, 22F, 23F, 33F, 35B, 15C, 23A, and 24F;
i) 1, 3, 4, 5, 6A, 6B, 7F, 8, 9N, 9V, 10A, 11A, 12F, 14, 15B, 18C, 19A, 19F, 22F, 23F, 33F, 35B, 15C, 23B, and 24F; or
j) 1, 3, 4, 5, 6A, 6B, 7F, 8, 9N, 9V, 10A, 11A, 12F, 14, 15B, 18C, 19A, 19F, 22F, 23F, 33F, 35B, 23A, 23B, and 24F.

In one aspect, the multivalent pneumococcal conjugate composition comprises pneumococcal capsular polysaccharide-protein conjugates, wherein the pneumococcal capsular polysaccharide-protein conjugates comprise or consist of 24 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae serotypes are selected from 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B. This multivalent pneumococcal conjugate composition is also referred to as PCV-24. In certain embodiments PCV-24, at least one of the Streptococcus pneumoniae serotypes is 35B. In certain embodiments of PCV-24, the Streptococcus pneumoniae serotypes comprise or consist of 1, 3, 4, 5, 6A, 6B, 7F, 8, 9N, 9V, 10A, 11A, 12F, 14, 15B, 18C, 19A, 19F, 22F, 23F, 33F, and 35B and two serotypes selected from 15A, 15C, 23A, 23B, and 24F. For example, the PCV-24 comprises pneumococcal capsular polysaccharide-protein conjugates, wherein the pneumococcal capsular polysaccharide-protein conjugates may comprise or consist of 24 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae serotypes are:
a) 1, 3, 4, 5, 6A, 6B, 7F, 8, 9N, 9V, 10A, 11A, 12F, 14, 15B, 18C, 19A, 19F, 22F, 23F, 33F, 35B, 15A, and 15C;
b) 1, 3, 4, 5, 6A, 6B, 7F, 8, 9N, 9V, 10A, 11A, 12F, 14, 15B, 18C, 19A, 19F, 22F, 23F, 33F, 35B, 15A and 23A;
c) 1, 3, 4, 5, 6A, 6B, 7F, 8, 9N, 9V, 10A, 11A, 12F, 14, 15B, 18C, 19A, 19F, 22F, 23F, 33F, 35B, 15A, and 23B;
d) 1, 3, 4, 5, 6A, 6B, 7F, 8, 9N, 9V, 10A, 11A, 12F, 14, 15B, 18C, 19A, 19F, 22F, 23F, 33F, 35B, 15A, and 24F;
e) 1, 3, 4, 5, 6A, 6B, 7F, 8, 9N, 9V, 10A, 11A, 12F, 14, 15B, 18C, 19A, 19F, 22F, 23F, 33F, 35B, 15C, and 23A;
f) 1, 3, 4, 5, 6A, 6B, 7F, 8, 9N, 9V, 10A, 11A, 12F, 14, 15B, 18C, 19A, 19F, 22F, 23F, 33F, 35B, 15C, and 23B;
g) 1, 3, 4, 5, 6A, 6B, 7F, 8, 9N, 9V, 10A, 11A, 12F, 14, 15B, 18C, 19A, 19F, 22F, 23F, 33F, 35B, 15C, and 24F;
h) 1, 3, 4, 5, 6A, 6B, 7F, 8, 9N, 9V, 10A, 11A, 12F, 14, 15B, 18C, 19A, 19F, 22F, 23F, 33F, 35B, 23A, and 23B;
i) 1, 3, 4, 5, 6A, 6B, 7F, 8, 9N, 9V, 10A, 11A, 12F, 14, 15B, 18C, 19A, 19F, 22F, 23F, 33F, 35B, 23A, and 24F; or
j) 1, 3, 4, 5, 6A, 6B, 7F, 8, 9N, 9V, 10A, 11A, 12F, 14, 15B, 18C, 19A, 19F, 22F, 23F, 33F, 35B, 23B, and 24F.

In one aspect, the multivalent pneumococcal conjugate composition comprises pneumococcal capsular polysaccharide-protein conjugates, wherein the pneumococcal capsular polysaccharide-protein conjugates comprise or consist of 23 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae serotypes are selected from 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B. This multivalent pneumococcal conjugate composition is also referred to as PCV-23. In certain embodiments of PCV-23, at least one of the Streptococcus pneumoniae serotypes is 35B. In certain embodiments of PCV-23, the Streptococcus pneumoniae serotypes comprise or consist of 1, 3, 4, 5, 6A, 6B, 7F, 8, 9N, 9V, 10A, 11A, 12F, 14, 15B, 18C, 19A, 19F, 22F, 23F, 33F, and 35B and one serotype selected from 15A, 15C, 23A, 23B, and 24F. For example, the PCV-23 comprises pneumococcal capsular polysaccharide-protein conjugates, wherein the pneumococcal capsular polysaccharide-protein conjugates may comprise or consist of 23 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae serotypes are:
a) 1, 3, 4, 5, 6A, 6B, 7F, 8, 9N, 9V, 10A, 11A, 12F, 14, 15B, 18C, 19A, 19F, 22F, 23F, 33F, 35B, and 15A;
b) 1, 3, 4, 5, 6A, 6B, 7F, 8, 9N, 9V, 10A, 11A, 12F, 14, 15B, 18C, 19A, 19F, 22F, 23F, 33F, 35B, and 15C;
c) 1, 3, 4, 5, 6A, 6B, 7F, 8, 9N, 9V, 10A, 11A, 12F, 14, 15B, 18C, 19A, 19F, 22F, 23F, 33F, 35B, and 23A;
d) 1, 3, 4, 5, 6A, 6B, 7F, 8, 9N, 9V, 10A, 11A, 12F, 14, 15B, 18C, 19A, 19F, 22F, 23F, 33F, 35B, and 23B; or
e) 1, 3, 4, 5, 6A, 6B, 7F, 8, 9N, 9V, 10A, 11A, 12F, 14, 15B, 18C, 19A, 19F, 22F, 23F, 33F, 35B, and 24F.

In one aspect, the multivalent pneumococcal conjugate composition comprises pneumococcal capsular polysaccharide-protein conjugates, wherein the pneumococcal capsular polysaccharide-protein conjugates comprise or consist of 22 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae serotypes are selected from 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B. This multivalent pneumococcal conjugate composition is also referred to as PCV-22. For example, the PCV-22 comprises pneumococcal capsular polysaccharide-protein conjugates, wherein the pneumococcal capsular polysaccharide-protein conjugates may comprise or consist of 22 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae serotypes are:
a) 1, 3, 4, 5, 6A, 6B, 7F, 8, 9N, 9V, 10A, 11A, 12F, 14, 15B, 18C, 19A, 19F, 22F, 23F, 33F, and 15A;
b) 1, 3, 4, 5, 6A, 6B, 7F, 8, 9N, 9V, 10A, 11A, 12F, 14, 15B, 18C, 19A, 19F, 22F, 23F, 33F, and 15C;
c) 1, 3, 4, 5, 6A, 6B, 7F, 8, 9N, 9V, 10A, 11A, 12F, 14, 15B, 18C, 19A, 19F, 22F, 23F, 33F, and 23A;
d) 1, 3, 4, 5, 6A, 6B, 7F, 8, 9N, 9V, 10A, 11A, 12F, 14, 15B, 18C, 19A, 19F, 22F, 23F, 33F, and 23B;
e) 1, 3, 4, 5, 6A, 6B, 7F, 8, 9N, 9V, 10A, 11A, 12F, 14, 15B, 18C, 19A, 19F, 22F, 23F, 33F, and 24F; or
f) 1, 3, 4, 5, 6A, 6B, 7F, 8, 9N, 9V, 10A, 11A, 12F, 14, 15B, 18C, 19A, 19F, 22F, 23F, 33F, and 35B.

The PCV-22, PCV-23, PCV-24, PCV-25, PCV-26, and PCV-27 embodiments can also include Streptococcus pneumoniae serotypes of interest other than serotypes 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B. For example, in certain embodiments, PCV-22, PCV-23, PCV-24, PCV-25, PCV-26, or PCV-27 further comprise one or more of Streptococcus pneumoniae serotypes 2, 12A, 16F, 17F, 20A, 20B, 20F, 31, 45, and 46. In certain embodiments, PCV-22, PCV-23, PCV-24, PCV-25, PCV-26, or PCV-27 further comprise one or more of Streptococcus pneumoniae serotypes 6C, 6D, 7B, 7C, 18B, 21, 22A, 24B, 27, 28A, 34, 35F, 38, and 39. Other Streptococcus pneumoniae serotypes of interest may also be added to any one of PCV-22, PCV-23, PCV-24, PCV-25, PCV-26, or PCV-27.

It is also possible to replace one or more of the Streptococcus pneumoniae serotypes of PCV-22, PCV-23, PCV-24, PCV-25, PCV-26, or PCV-27 with one or more Streptococcus pneumoniae serotypes of interest other than serotypes 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B. For example, in certain embodiments, one or more of serotypes 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B in PCV-22, PCV-23, PCV-24, PCV-25, PCV-26, or PCV-27 is replaced with one or more of Streptococcus pneumoniae serotypes 2, 12A, 14, 16F, 20A, 20B, 20F, 31, 45, and 46. In certain embodiments, one or more of serotypes 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B in PCV-22, PCV-23, PCV-24, PCV-25, PCV-26, or PCV-27 is replaced with one or more of Streptococcus pneumoniae serotypes 6C, 6D, 7B, 7C, 18B, 21, 22A, 24B, 27, 28A, 34, 35F, 38, and 39. Other Streptococcus pneumoniae serotypes of interest may also be used replace one or more of serotypes 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B in any one of PCV-22, PCV-23, PCV-24, PCV-25, PCV-26, or PCV-27.

Carrier Protein

In a polysaccharide-protein conjugate vaccine, a carrier protein is conjugated to a polysaccharide antigen to form a glycoconjugate. The carrier protein helps to enhance the immune response (e.g. antibody response) to the polysaccharide antigen. Carrier proteins should be amenable to conjugation with a pneumococcal polysaccharide using standard conjugation procedures.

Carrier proteins that can be used in the glycoconjugate include, but are not limited to, DT (diphtheria toxoid), TT (tetanus toxoid), fragment C of TT, CRM₁₉₇ (a genetically derived non-toxic variant of diphtheria toxin that retains the immunologic properties of the wild type diphtheria toxin), other genetically derived diphtheria toxin variants (for example, CRM176, CRM228, CRM45 (Uchida et al. (1973) J. Biol. Chem. 218:3838-3844), CRM9, CRM102, CRM103 or CRM107; and other mutations described by Nicholls and Youle in Genetically Engineered Toxins, Ed: Frankel, Marcel Dekker Inc. (1992); deletion or mutation of Glu-148 to Asp, Gln or Ser and/or Ala 158 to Gly and other mutations disclosed in U.S. Pat. Nos. 4,709,017 and 4,950,740; mutation of at least one or more residues Lys 516, Lys 526, Phe 530 and/or Lys 534 and other mutations disclosed in U.S. Pat. Nos. 5,917,017 and 6,455,673; or fragment disclosed in U.S. Pat. No. 5,843,711, pneumococcal pneumolysin (ply) (Kuo et al. (1995) Infect Immun 63:2706-2713) including ply detoxified in some fashion, for example dPLY-GMBS (WO 2004/081515, WO 2006/032499) or dPLY-formol, PhtX, including PhtA, PhtB, PhtD, PhtE (sequences of PhtA, PhtB, PhtD or PhtE are disclosed in WO 00/37105 and WO 00/39299) and fusions of Pht proteins, for example PhtDE fusions, PhtBE fusions, Pht A-E (WO 01/98334, WO 03/054007, WO 2009/000826), OMPC (meningococcal outer membrane protein), which is usually extracted from Neisseria meningitidis serogroup B (EP0372501), PorB (from N. meningitidis), PD (Haemophilus influenzae protein D; see, e.g., EP0594610 B), or immunologically functional equivalents thereof, synthetic peptides (EP0378881, EP0427347), heat shock proteins (WO 93/17712, WO 94/03208), pertussis proteins (WO 98/58668, EP0471177), cytokines, lymphokines, growth factors or hormones (WO 91/01146), artificial proteins comprising multiple human CD4+ T cell epitopes from various pathogen derived antigens (Falugi et al. (2001) Eur J Immunol 31:3816-3824) such as N19 protein (Baraldoi et al. (2004) Infect Immun 72:4884-4887) pneumococcal surface protein PspA (WO 02/091998), iron uptake proteins (WO 01/72337), toxin A or B of Clostridium difficile (WO 00/61761), transferrin binding proteins, pneumococcal adhesion protein (PsaA), recombinant Pseudomonas aeruginosa exotoxin A (in particular non-toxic mutants thereof (such as exotoxin A bearing a substitution at glutamic acid 553 (Douglas et al. (1987) J Bacteriol. 169(11):4967-4971)). Other proteins, such as ovalbumin, keyhole limpet hemocyanin (KLH), bovine serum albumin (BSA) or purified protein derivative of tuberculin (PPD) also can be used as carrier proteins. Other suitable carrier proteins include inactivated bacterial toxins such as cholera toxoid (e.g., as described in WO 2004/083251), Escherichia coli LT, E. coli ST, and exotoxin A from P. aeruginosa, and immunological functional equivalents thereof may also be used as carrier proteins in the present invention. When each of the carrier proteins is referred to in the present specification, they are understood to encompass their immunological functional equivalents.

In certain embodiments, the carrier protein of the glycoconjugate is selected from the group consisting of TT (including fragment C of TT), DT (including DT variants, such as CRM₁₉₇ and the others discussed above), PD, PhtX, PhtD, PhtDE fusions (especially those disclosed in WO 01/98334 and WO 03/054007), detoxified pneumolysin, PorB, N19 protein, PspA, OMPC, toxin A or B of Clostridium difficile, and PsaA. When each of the carrier proteins is referred to in the present specification, they are understood to encompass their immunological functional equivalents. It will be appreciated by those skilled in the art that, for example, DT mutants, which are immunologically functional equivalents thereof, including but not limited to those discussed above, are also included when DT is referred to in the specification.

In certain embodiments, the carrier protein of the glycoconjugate is selected from the group consisting of DT (Diphtheria toxoid), CRM₁₉₇, TT (Tetanus toxoid), fragment C of TT and PD (protein D of Haemophilus influenza).

In one embodiment, the carrier protein of the glycoconjugate of the invention may be DT (diphtheria toxoid). Naturally occurring or wild-type diphtheria toxins can be obtained from toxin-producing strains available from various public sources including the American Type Culture Collection (ATCC). As used herein, the term DT (diphtheria toxoid) is meant to include all DT variants that function as functional equivalents thereof. Such DT mutants include, for example, CRM176, CRM228, CRM45, CRM9, CRM102, CRM103 or CRM107; Mutation or deletion of Glu148 to Asp as compared to wild type DT (disclosed in U.S. Pat. No. 4,709,017); deletion or mutation of Glu148 to Asp, deletion or mutation of Ala158 to Gly disclosed in U.S. Pat. Nos. 4,709,017 and 4,950,740; mutation of at least one or more residues selected from the group consisting of Lys 516, Lys 526, Phe 530 and Lys 534 disclosed in U.S. Pat. No. 5,917,017 and mutation of Glu148, Glu349, Lys516 or/and Phe530 disclosed in U.S. Pat. No. 6,455,673; or in U.S. Pat. No. 5,843,711, and the like, but are not limited thereto. In one embodiment, the isolated capsular saccharide is conjugated to the CRM₁₉₇ protein. The CRM₁₉₇ protein is a non-toxic form of the diphtheria toxin that retains the immunologic properties of the wild type diphtheria toxin. The CRM₁₉₇ is produced by Corynebacterium diphtheriae infected by the nontoxigenic phage β₁₉₇tox-created by nitrosoguanidine mutagenesis of the toxigenic corynephage beta (Uchida et al. (1971) Nature New Biology 233:8-11). The CRM₁₉₇ protein has the same molecular weight as the diphtheria toxin but differs therefrom by a single base change (guanine to adenine) in the structural gene. This single base change causes an amino acid substitution (glutamic acid for glycine) in the mature protein and eliminates the toxic properties of diphtheria toxin. The CRM₁₉₇ protein is a safe and effective T-cell dependent carrier for saccharides. Further details about CRM₁₉₇ and production thereof can be found, e.g., in U.S. Pat. No. 5,614,382, which is hereby incorporated by reference in its entirety.

In another embodiment, the carrier protein of the glycoconjugate is a TT (tetanus toxoid). Tetanus toxoid is prepared and used worldwide for large-scale immunization against tetanus (or lockjaw) caused by Clostridium tetani. Tetanus toxoid is also used both singly and in combination with diphtheria and/or pertussis vaccines. The parent protein, tetanus toxin, is generally obtained in cultures of Clostridium tetani. Tetanus toxin is a protein of about 150 kDa and consists of two subunits (about 100 kDa and about 50 kDa) linked by a disulfide bond. The toxin is typically detoxified with formaldehyde and can be purified from culture filtrates using known methods, such as ammonium sulfate precipitation (see, e.g., Levin and Stone, J Immunol., 67:235-242 (1951); W.H.O. Manual for the Production and Control of Vaccines: Tetanus Toxoid, 1977 (BLG/UNDP/77.2 Rev.I.)) or chromatography techniques, as disclosed, for example, in WO 1996/025425. Tetanus toxin may also be inactivated by recombinant genetic means.

In another embodiment, the carrier protein of the glycoconjugate may be PD (Protein D of Haemophilus influenza; see, e.g., EP 0594610B).

In certain embodiments, a single carrier protein is used in the multivalent pneumococcal conjugate composition. In certain embodiments, more than one protein carrier are used (“mixed carrier”). In these mixed carrier embodiments, 2, 3, 4, 5, 6, 7, 8, 9, or more carrier proteins can be used. Typically, the mixed carrier embodiments, include two carrier proteins. For example, in certain embodiments, certain capsular polysaccharides are conjugated to a first protein carrier and the remaining capsular polysaccharides are attached to a second protein carrier.

In one aspect, the first protein carrier is CRM₁₉₇ and the second protein carrier is tetanus toxoid. In certain embodiments, two of the capsular polysaccharides are conjugated to tetanus toxoid and the remaining capsular polysaccharides are conjugated to CRM₁₉₇. In certain embodiments, the two capsular polysaccharides that are conjugated to tetanus toxoid are selected from the group consisting of serotypes 1, 3, and 5. In certain embodiments, four of the capsular polysaccharides are conjugated to tetanus toxoid and the remaining capsular polysaccharides are conjugated to CRM₁₉₇. In certain embodiments, the four capsular polysaccharides that are conjugated to tetanus toxoid are selected from the group consisting of serotypes 1, 3, 5, 15B, and 22F. In certain embodiments, the four capsular polysaccharides that are conjugated to tetanus toxoid are serotypes 1, 5, 15B, and 22F; serotypes 1, 3, 15B, and 22F; or serotypes 3, 5, 15B, and 22F.

In some embodiments of PCV-22, PCV-23, PCV-24, PCV-25, PCV-26, or PCV-27, the capsular polysaccharides from serotypes 1 and 5 are conjugated to tetanus toxoid, and the capsular polysaccharides from the remaining serotypes are conjugated to CRM₁₉₇. In another embodiment of PCV-22, PCV-23, PCV-24, PCV-25, PCV-26, or PCV-27, the capsular polysaccharides from serotypes 1 and 3 are conjugated to tetanus toxoid, and the remaining capsular polysaccharides are conjugated to CRM₁₉₇. In another embodiment of PCV-22, PCV-23, PCV-24, PCV-25, PCV-26, or PCV-27, the capsular polysaccharides from serotypes 3 and 5 are conjugated to tetanus toxoid, and the remaining capsular polysaccharides are conjugated to CRM₁₉₇. In another embodiment of PCV-22, PCV-23, PCV-24, PCV-25, PCV-26, or PCV-27, the capsular polysaccharides from serotypes 1, 5, 15B, and 22F are conjugated to tetanus toxoid, and the remaining capsular polysaccharides are conjugated to CRM₁₉₇. In another embodiment of PCV-22, PCV-23, PCV-24, PCV-25, PCV-26, or PCV-27, the capsular polysaccharides from serotypes 1, 3, 15B, and 22F are conjugated to tetanus toxoid, and the remaining capsular polysaccharides are conjugated to CRM₁₉₇. In another embodiment of PCV-22, PCV-23, PCV-24, PCV-25, PCV-26, or PCV-27, the capsular polysaccharides from serotypes 3, 5, 15B, and 22F are conjugated to tetanus toxoid, and the remaining capsular polysaccharides are conjugated to CRM₁₉₇.

The pneumococcal capsular polysaccharides used in the compositions and vaccines described herein, including the capsular polysaccharides from serotypes 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B, may be prepared from Streptococcus pneumoniae using any available technique, including standard techniques known to one of ordinary skill in the art, including, for example, those disclosed in WO 2006/110381, WO 2008/118752, WO 2006/110352, and U.S. Patent App. Pub. Nos. 2006/0228380, 2006/0228381, 2007/0184071, 2007/0184072, 2007/0231340, 2008/0102498 and 2008/0286838, all of which are incorporated by reference in their entireties. For example, each pneumococcal capsular polysaccharide serotype may be grown in culture medium (e.g., a soy-based medium). The cells are lysed, and individual polysaccharides may be purified from the lysate through centrifugation, precipitation, ultra-filtration, and/or column chromatography. In addition, the pneumococcal capsular oligosaccharides can be produced using synthetic protocols.

Capsular polysaccharides of Streptococcus pneumoniae comprise repeating oligosaccharide units, which may contain up to 8 sugar residues. A capsular saccharide antigen may be a full length polysaccharide, or it may be reduced in size (e.g., a single oligosaccharide unit, or a shorter than native length saccharide chain of repeating oligosaccharide units). The size of capsular polysaccharides may be reduced by various methods known in the art, such as acid hydrolysis treatment, hydrogen peroxide treatment, sizing by a high pressure homogenizer, optionally followed by a hydrogen peroxide treatment to generate oligosaccharide fragments, or microfluidization. In certain embodiments, prior to reacting the purified capsular polysaccharide with an oxidizing agent to produce an activated capsular polysaccharide, the purified capsular polysaccharide is subjected to a sizing step, such as acid hydrolysis treatment or microfluidization, to reduce its size. In certain embodiments, the capsular polysaccharide is not subjected to a sizing step, such as acid hydrolysis treatment or microfluidization, prior to reacting the purified capsular polysaccharide with an oxidizing agent to produce an activated capsular polysaccharide.

The pneumococcal conjugate of each of the serotypes may be prepared by conjugating a capsular polysaccharide of each serotype to a carrier protein. The different pneumococcal conjugates may be formulated into a composition, including a single dosage formulation.

Activation of Capsular Polysaccharide

To prepare a polysaccharide-protein conjugate, the capsular polysaccharides prepared from each pneumococcal serotype may be chemically activated so that the capsular polysaccharides may react with a carrier protein. Once activated, each capsular polysaccharide may be separately conjugated to a carrier protein to form a glycoconjugate. The chemical activation of the polysaccharides and subsequent conjugation to the carrier protein may be achieved by conventional methods.

For example, vicinal hydroxyl groups at the end of the capsular polysaccharides can be oxidized to aldehyde groups by oxidizing agents such as periodates (including sodium periodate, potassium periodate, or periodic acid), as disclosed, for example, in U.S. Pat. Nos. 4,365,170, 4,673,574 and 4,902,506, which are hereby incorporated by reference in their entireties. The periodate randomly oxidizes the vicinal hydroxyl group of a carbohydrate to form a reactive aldehyde group and causes cleavage of a C—C bond. The term “periodate” includes both periodate and periodic acid. This term also includes both metaperiodate (IO₄₋) and orthoperiodate (IO₆⁵⁻). The term “periodate” also includes various salts of periodate including sodium periodate and potassium periodate. In certain embodiments, the polysaccharide may be oxidized in the presence of sodium metaperiodate.

In certain embodiments, the periodate may be used in an amount of about 0.03-0.17 μg per 1 μg of polysaccharide. In certain embodiments, the periodate may be used in an amount of about 0.025-0.18 μg or about 0.02-0.19 μg per 1 μg of polysaccharide. The saccharide may be activated as desired within the above range. Outside the range, the effect may be unsatisfactory.

Polysaccharides may also be activated with 1-cyano-4-dimethylamino pyridinium tetrafluoroborate (CDAP) to form a cyanate ester. The activated polysaccharide is then coupled directly or via a spacer or linker group to an amino group on the carrier protein.

For example, the spacer could be cystamine or cysteamine to give a thiolated polysaccharide which could be coupled to the carrier via a thioether linkage obtained after reaction with a maleimide-activated carrier protein (for example using N-[y-maleimidobutyrloxy]succinimide ester (GMBS)) or a haloacetylated carrier protein (for example using iodoacetimide, N-succinimidyl bromoacetate (SBA; SIB), N-succinimidyl(4-iodoacetyl)aminobenzoate (SlAB), sulfosuccinimidyl(4-iodoacetyl)aminobenzoate (sulfo-SIAB), N-succinimidyl iodoacetate (SIA) or succinimidyl 3-[bromoacetamido]proprionate (SBAP)). Preferably, the cyanate ester (optionally made by COAP chemistry) is coupled with hexane diamine or adipic acid dihydrazide (AOH) and the amino-derivatized saccharide is conjugated to the carrier protein using carbodiimide (e.g., EDAC or EDC) chemistry via a carboxyl group on the protein carrier. Such conjugates are described for example in WO 93/15760, WO 95/08348 and WO 96/129094, all of which are hereby incorporated by reference in their entireties.

After the activation step, the activated capsular polysaccharide is optionally lyophilized before mixing the activated polysaccharide with the carrier protein. The activated polysaccharide and the carrier protein may be lyophilized separately or may be combined with each other and then lyophilized.

The activated capsular polysaccharide may be lyophilized in the presence of any cryoprotectant, such as a saccharide. For example, the saccharide may be selected from, but is not limited to, sucrose, trehalose, raffinose, stachiose, melezitose, dextran, mannitol, lactitol and palatinit. In certain embodiments, the saccharide is sucrose. The lyophilized polysaccharide is then resuspended in a solvent before the conjugation reaction. The lyophilized activated capsular polysaccharides may be mixed with a solution comprising a carrier protein. Alternatively, the co-lyophilized polysaccharide and carrier protein are resuspended in a solvent before the conjugation reaction.

Conjugation of Activated Capsular Polysaccharide to Carrier Protein

The conjugation of the activated capsular polysaccharides and the carrier proteins may be achieved, for example, by reductive amination, as described, for example, in U.S. Patent Appl. Pub. Nos. 2006/0228380, 2007/0231340, 2007/0184071 and 2007/0184072, WO 2006/110381, WO 2008/079653, and WO 2008/143709, all of which are incorporated by reference in their entireties. For example, the activated capsular polysaccharides and the carrier protein may be reacted with a reducing agent to form a conjugate. Reducing agents which are suitable include borohydrides, such as sodium cyanoborohydride, borane-pyridine, sodium triacetoxyborohydride, sodium or borohydride, or borohydride exchange resin. At the end of the reduction reaction, there may be unreacted aldehyde groups remaining in the conjugates. The unreacted aldehyde groups may be capped using a suitable capping agent, such as sodium borohydride (NaBH₄). In an embodiment, the reduction reaction is carried out in aqueous solvent. In another embodiment the reaction is carried out in aprotic solvent. In an embodiment, the reduction reaction is carried out in DMSO (dimethylsulfoxide) or in DMF (dimethylformamide) solvent. Other possible reducing agents include, but are not limited to, amine-boranes such as pyridine-borane, 2-picoline-borane, 2,6-diborane-methanol, dimethylamine-borane, t-BuMeiPrN-BH3, benzylamine-BH3 or 5-ethyl-2-methylpyridine-borane (PEMB).

The activated capsular polysaccharides may be conjugated directly to the carrier protein or indirectly through the use of a spacer or linker, such as a bifunctional linker. The linker is optionally heterobifunctional or homobifunctional, having for example a reactive amino group and a reactive carboxylic acid group, 2 reactive amino groups or two reactive carboxylic acid groups.

Other suitable techniques for conjugation use carbodiimides, hydrazides, active esters, norborane, p-nitrobenzoic acid, N-hydroxysuccinimide, S-NHS, EDC, TSTU, as described, for example, in International Patent Application Publication No. WO 98/42721, which is incorporated by reference in their entirety. Conjugation may involve a carbonyl linker which may be formed by reaction of a free hydroxyl group of the saccharide with 1,1′-carbonyldiimidazole (CDl) (see Bethell et al. (1979) J Biol. Chem. 254:2572-2574; Hearn et al. (1981) J Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDl carbamate intermediate with an amino group on a protein.

The ratio of polysaccharide to carrier protein for pneumococcal conjugate vaccines is typically in the range 0.3-3.0 (w/w) but can vary with the serotype. The ratio can be determined either by independent measurement of the amounts of protein and polysaccharide present, or by methods that give a direct measure of the ratio known in the art. Methods including ¹H NMR spectroscopy or SEC-HPLC-UV/RI with dual monitoring (e.g. refractive index and UV, for total material and protein content respectively) can profile the saccharide/protein ratio over the size distribution of conjugates, as well as by SEC-HPLC-MALLS or MALDI-TOF-MS.

The polysaccharide-protein conjugates thus obtained may be purified and enriched by a variety of methods. These methods include concentration/diafiltration, column chromatography, and depth filtration. The purified polysaccharide-protein conjugates are combined to formulate the multivalent pneumococcal conjugate composition, which can be used as a vaccine.

Formulation

Formulation of a vaccine composition can be accomplished using art-recognized methods. A vaccine composition is formulated to be compatible with its intended route of administration. The individual pneumococcal capsular polysaccharide-protein conjugates can be formulated together with a physiologically acceptable vehicle to prepare the composition. Examples of such vehicles include, but are not limited to, water, buffered saline, polyols (e.g., glycerol, propylene glycol, liquid polyethylene glycol) and dextrose solutions.

In some embodiments, the multivalent pneumococcal conjugate composition further comprises an adjuvant. As used herein, an “adjuvant” refers to a substance or vehicle that non-specifically enhances the immune response to an antigen. Adjuvants can include but are not limited to, the following:

(1) aluminum salts (alum), such as aluminum hydroxide, aluminum phosphate, aluminum sulfate, aluminum hydroxy phosphate sulfate, etc.;

(2) oil-in-water emulsion formulations (with or without other specific immunostimulating agents such as muramyl peptides (defined below) or bacterial cell wall components), such as, for example, (a) MF59 (WO 90/14837), containing 5% Squalene, 0.5% Tween 80, and 0.5% Span 85 (optionally containing various amounts of MTP-PE (see below), although not required) formulated into submicron particles using a microfluidizer such as Model 110Y microfluidizer (Microfluidics, Newton, Mass.), (b) SAF, containing 10% Squalene, 0.4% Tween 80, 5% pluronic-blocked polymer L121, and thr-MDP (see below) either microfluidized into a submicron emulsion or vortexed to generate a larger particle size emulsion, and (c) Ribi™ adjuvant system (RAS), (Corixa, Hamilton, Mont.) containing 2% Squalene, 0.2% Tween 80, and one or more bacterial cell wall components from the group consisting of 3-O-deaylated monophosphorylipid A (MPL™) described in U.S. Pat. No. 4,912,094 (Corixa), trehalose dimycolate (TDM), and cell wall skeleton (CWS), preferably MPL+CWS (Detox™);

(3) saponin adjuvants, such as Quil A or STIMULON™ QS-21 (Antigenics, Framingham, Mass.) (U.S. Pat. No. 5,057,540) or particles generated therefrom such as ISCOMs (immunostimulating complexes);

(4) bacterial lipopolysaccharides, synthetic lipid A analogs such as aminoalkyl glucosamine phosphate compounds (AGP), or derivatives or analogs thereof, which are available from Corixa, and which are described in U.S. Pat. No. 6,113,918; one such AGP is 2-[(R)-3-Tetradecanoyloxytetradecanoylamino]ethyl 2-Deoxy-4-O-phosphono-3-0-[(R)-3-tetradecanoyloxytetradecanoy]-2-[(R)-3-tetr adecanoyloxytetradecanoylamino]-b-D-glucopyranoside, which is also known as 529 (formerly known as RC529), which is formulated as an aqueous form or as a stable emulsion,

(5) synthetic polynucleotides such as oligonucleotides containing CpG motif(s) (U.S. Pat. No. 6,207,646);

(6) cytokines, such as interleukins (e.g., IL-1, IL-2, IL-4, IL-5, IL-6, IL-7, IL-12, IL-15, IL-18, etc.), interferons (e.g., gamma interferon), granulocyte macrophage colony stimulating factor (GM-CSF), macrophage colony stimulating factor (M-CSF), tumor necrosis factor (TNF), costimulatory molecules B7-1 and B7-2, etc.;

(7) detoxified mutants of a bacterial ADP-ribosylating toxin such as a cholera toxin (CT) either in a wild-type or mutant form, for example, where the glutamic acid at amino acid position 29 is replaced by another amino acid, preferably a histidine, in accordance with WO 00/18434 (see also WO 02/098368 and WO 02/098369), a pertussis toxin (PT), or an E. coli heat-labile toxin (LT), particularly LT-K63, LT-R72, CT-S109, PT-K9/G129 (see, e.g., WO 93/13302 and WO 92/19265); and

(8) complement components such as trimer of complement component C3d;

(9) biological molecules, such as lipids and costimulatory molecules. Exemplary biological adjuvants include AS04, IL-2, RANTES, GM-CSF, TNF-α, IFN-γ, G-CSF, LFA-3, CD72, B7-1, B7-2, OX-40L and 41 BBL.

Muramyl peptides include, but are not limited to, N-acetyl-muramyl-L-threonyl-D-isoglutamine (thr-MDP), N-acetyl-normuramyl-L-alanine-2-(1′-2′dipalmitoyl-sn-glycero-3-hydroxyphosphoryloxy)-ethylamine (MTP-PE), etc.

The adjuvant is appropriately selected according to the amount and valence of the conjugate in the composition. In some embodiments, the adjuvant is an aluminum-based adjuvant. When using an aluminum-based adjuvant, the aluminum element in the composition based on the aluminum element may be added to comprise 0.01 mg/mL to 1 mg/mL. Typically, a single 0.5 ml vaccine dose is formulated to contain about 0.1 mg to 2.5 mg of the aluminum-based adjuvant. In other embodiments, a single 0.5 ml vaccine dose is formulated to contain between 0.1 mg to 2 mg, 0.1 mg to 1 mg, 0.1 mg to 0.5 mg, 0.1 mg to 0.2 mg, 0.125 mg to 2.5 mg, 0.125 mg to 0.5 mg, 0.125 mg to 0.2 mg or 0.125 to 0.25 mg of the aluminum-based adjuvant. In certain embodiments, a single 0.5 ml vaccine dose is formulated to contain about 0.125 mg to about 0.250 mg of the aluminum-based adjuvant. In certain embodiments, a single 0.5 ml vaccine dose is formulated to contain about 0.125 mg of the aluminum-based adjuvant. In certain embodiments, a single 0.5 ml vaccine dose is formulated to contain about 0.250 mg of the aluminum-based adjuvant.

In particular embodiments, the adjuvant is selected from the group consisting of aluminum phosphate, aluminum sulfate, and aluminum hydroxide.

In particular embodiments, the adjuvant is aluminum phosphate.

In some embodiments, the composition is for use as a vaccine against an infection of Streptococcus pneumoniae.

### Characterization of Pneumococcal Capsular Polysaccharide-Protein Carrier Conjugates

In certain embodiments, the polysaccharide-protein carrier conjugate may have a molecular weight of 100-10,000 kDa. In certain embodiments, the conjugate has a molecular weight of 200-9,000 kDa. In certain embodiments, the conjugate has a molecular weight of 300-8,000 kDa. In certain embodiments, the conjugate has a molecular weight of 400-7,000 kDa. In certain embodiments, the conjugate has a molecular weight of 500-6,000 kDa. In certain embodiments, the conjugate has a molecular weight of 600-5,000 kDa. In certain embodiments, the conjugate has a molecular weight of 500-4,000 kDa molecular weight. Any whole number within any of the above ranges is contemplated as an embodiment of the present disclosure.

When the molecular weight is within the above range, the conjugate may be formed stably with high yield. Also, the proportion of a free polysaccharide can be reduced. In addition, superior immunogenicity can be achieved within the above molecular weight range.

After the individual polysaccharide-protein conjugates are purified, they are compounded to formulate the immunogenic composition of the present disclosure.

The saccharide-protein conjugates of the serotypes of the present disclosure may be characterized by a ratio of the polysaccharide to the protein carrier (amount of polysaccharide/amount of protein carrier, w/w).

In certain embodiments, the ratio (w/w) of the polysaccharide to the protein carrier in the polysaccharide-protein carrier conjugate for each serotype is 0.5-2.5, 0.4-2.3, 0.3-2.1, 0.24-2, 0.2-1.8, 0.18-1.6, 0.16-1.4, 0.14-1.2, 0.12-1, 0.1-1, 0.4-1.3, 0.5-1, or 0.7-0.9 (e.g., about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4 or about 2.5).

When the ratio of the polysaccharide to the protein carrier is within the above range, the conjugate may be formed stably with high yield. Also, the proportion of a free polysaccharide can be reduced. In addition, superior immunogenicity can be achieved and the conjugate can be maintained stably without interference by other serotypes within the above range.

The conjugates and immunogenic compositions of the present disclosure may contain a free polysaccharide which is not covalently conjugated to the protein carrier but is nevertheless present in the polysaccharide-protein carrier conjugate composition. The free polysaccharide may be non-covalently associated with the polysaccharide-protein carrier conjugate (i.e., non-covalently bound to, adsorbed to, or entrapped in or by the polysaccharide-protein carrier conjugate).

In certain embodiments, the polysaccharide-protein carrier conjugate contains less than about 60%, about 50%, 45%, 40%, 35%, 30%, 25%, 20% or 15% of a free polysaccharide of each serotype based on the total amount of the polysaccharide of each serotype. In certain embodiments, the polysaccharide-protein carrier conjugate of each serotype contains less than about 60% of a free polysaccharide of each serotype based on the total amount of the polysaccharide of each serotype. In certain embodiments, the polysaccharide-protein carrier conjugate of each serotype contains less than about 50% of a free polysaccharide of each serotype based on the total amount of the polysaccharide of each serotype In certain embodiments, the polysaccharide-protein carrier conjugate of each serotype contains less than about 40% of a free polysaccharide of each serotype based on the total amount of the polysaccharide of each serotype. In certain embodiments, the polysaccharide-protein carrier conjugate of each serotype contains less than about 30% of a free polysaccharide of each serotype based on the total amount of the polysaccharide of each serotype. In certain embodiments, the polysaccharide-protein carrier conjugate of each serotype contains less than about 25% of a free polysaccharide of each serotype based on the total amount of the polysaccharide of each serotype In certain embodiments, the polysaccharide-protein carrier conjugate of each serotype contains less than about 20% of a free polysaccharide of each serotype based on the total amount of the polysaccharide of each serotype. In certain embodiments, the polysaccharide-protein carrier conjugate of each serotype contains less than about 15% of a free polysaccharide of each serotype based on the total amount of the polysaccharide of each serotype In certain embodiments, the polysaccharide-protein carrier conjugate of each serotype contains less than about 10% of a free polysaccharide of each serotype based on the total amount of the polysaccharide of each serotype.

The polysaccharide-protein carrier conjugate of each serotype may also be characterized by its molecular size distribution (K_{d}). A size exclusion chromatography medium (CL-4B; cross-linked agarose beads, 4%) may be used to determine the relative molecular size distribution of the conjugate. Size exclusion chromatography (SEC) is used in a gravity-fed column to profile the molecular size distribution of the conjugate. Large molecules excluded from the pores in the medium are eluted more quickly than small molecules. A fraction collector is used to collect the column eluate. The fractions are tested colorimetrically by saccharide assay. For the determination of K_{d}, the column is calibrated to establish the fraction at which molecules are completely excluded (V₀; K_{d}=0) and the fraction representing the maximum retention (Vᵢ; K_{d}=1). The fraction at which a specified sample attribute is reached (Vₑ) is related to K_{d} by the expression K_{d}=(Vₑ−V₀)/(Vᵢ−V₀).

In certain embodiments, at least 15% of the polysaccharide-protein carrier conjugate of each serotype may have a K_{d} of 0.3 or below in a CL-4B column.

In certain embodiments, at least 20% of the polysaccharide-protein carrier conjugate of each serotype may have a K_{d} of 0.3 or below in a CL-4B column. In certain embodiments, at least 15%, 20%, 25%, 30%, 35%, 40%, 45%, 50%, 55%, 60%, 65%, 70%, 75%, 80%, 85% or 90% of the polysaccharide-protein carrier conjugate of each serotype may have a K_{d} of 0.3 or below in a CL-4B column. In certain embodiments, at least 60% of the polysaccharide-protein carrier conjugate of each serotype may have a K_{d} of 0.3 or below in a CL-4B column. In certain embodiments, at least 50-80% of the polysaccharide-protein carrier conjugate of each serotype may have a K_{d} of 0.3 or below in a CL-4B column. In certain embodiments, at least 65-80% of the polysaccharide-protein carrier conjugate of each serotype may have a K_{d} of 0.3 or below in a CL-4B column. In certain embodiments, at least 15-60% of the saccharide-protein conjugate of each serotype may have a K_{d} of 0.3 or below in a CL-4B column.

### Prophylactic Methods and Uses

In one aspect, this disclosure provides a vaccine comprising a multivalent pneumococcal conjugate composition (e.g., PCV-22, PCV-23, PCV-24, PCV-25, PCV-26, or PCV-27) and a pharmaceutically acceptable excipient. In some embodiments, the pharmaceutically acceptable excipient comprises at least a buffer, such as a succinate buffer, a salt, such as sodium chloride, and/or a surface active agent, such as a polyoxyethylene sorbitan ester (e.g., polysorbate 80).

In some embodiments, the vaccine elicits a protective immune response in a human subject against disease caused by Streptococcus pneumoniae infection.

According to a further aspect, this disclosure provides a method for prophylaxis of Streptococcus pneumoniae infection or disease, the method comprising administering to a human subject a prophylactically effective amount of a multivalent pneumococcal conjugate composition (e.g., PCV-22, PCV-23, PCV-24, PCV-25, PCV-26, or PCV-27) or a vaccine comprising the same. The multivalent pneumococcal conjugate composition (e.g., PCV-22, PCV-23, PCV-24, PCV-25, PCV-26, or PCV-27) or vaccine comprising the same may be administered by any route, including, for example, by a systemic or mucosal route, as described below in further detail.

In certain embodiments, the human subject is an elderly subject and the disease is pneumonia or invasive pneumococcal disease (IPD). In certain embodiments, the elderly subject is at least 50 years old. In other embodiments, the elderly subject is at least 55 years old. In yet other embodiments, the elderly subject is at least 60 years old.

In other embodiments, the human subject is an infant and the disease is pneumonia, invasive pneumococcal disease (IPD), or acute otitis media (AOM). In certain embodiments, the infant is 0-2 years. In other embodiments, the infant is 2 to 15 months.

In yet another embodiment, the human subject is 6 weeks to 17 years of age and the disease is pneumonia, invasive pneumococcal disease (IPD) or acute otitis media (AOM). In certain embodiments, the human subject is 6 weeks to 5 years of age. In other embodiments, the human subject is 5 to 17 years of age.

The amount of conjugate in each vaccine dose or the prophylactically effective amount of the mixed carrier, multivalent pneumococcal conjugate composition may be selected as an amount that induces prophylaxis without significant, adverse effects. Such an amount can vary depending upon the pneumococcal serotype. Generally, each dose may include about 0.1 μg to about 100 μg of polysaccharide, specifically, about 0.1 to 10 μg, and, more specifically, about 1 μg to about 5 μg. Optimal amounts of components for a particular vaccine can be ascertained by standard studies involving observation of appropriate immune responses in subjects. For example, the amount for vaccination of a human subject can be determined by extrapolating an animal test result. In addition, the dose can be determined empirically.

In some embodiments, the vaccine or the multivalent pneumococcal conjugate composition (e.g., PCV-22, PCV-23, PCV-24, PCV-25, PCV-26, or PCV-27) may be a single 0.5 ml dose formulated to contain about 1 μg to about 5 μg of each capsular polysaccharide; about 20 μg to about 85 μg of carrier protein (e.g., CRM₁₉₇); and optionally about 0.1 mg to about 0.5 mg of elemental aluminum adjuvant. In some embodiments, the vaccine or the multivalent pneumococcal conjugate composition (e.g., PCV-22, PCV-23, PCV-24, PCV-25, PCV-26, or PCV-27) may be a single 0.5 ml dose formulated to contain about 2 μg to about 2.5 μg of each capsular polysaccharide except serotype 6B and optionally serotype 3, which is/are present in an amount of about 4 μg to about 5 μg; about 40 μg to about 75 μg of protein carrier (e.g., CRM₁₉₇); and optionally about 0.1 mg to about 0.25 mg of elemental aluminum adjuvant.

In some embodiments, the vaccine or the mixed carrier, multivalent pneumococcal conjugate composition (e.g., PCV-22, PCV-23, PCV-24, PCV-25, PCV-26, or PCV-27) may be a single 0.5 ml dose formulated to contain about 1 μg to about 5 μg of each capsular polysaccharide; about 1 μg to about 30 μg of a first carrier protein (e.g., TT); about 20 μg to about 100 μg of a second carrier protein (e.g., CRM₁₉₇); and optionally about 0.1 mg to about 0.5 mg of elemental aluminum adjuvant.

In some embodiments, the vaccine or the mixed carrier, multivalent pneumococcal conjugate composition (e.g., PCV-22, PCV-23, PCV-24, PCV-25, PCV-26, or PCV-27) may be a single 0.5 ml dose formulated to contain about 2 μg to about 2.5 μg of each capsular polysaccharide except serotype 6B and optionally serotype 3, which is/are present in an amount of about 4 μg to about 5 μg; about 2 μg to about 25 μg of a first carrier protein (e.g., TT); about 40 μg to about 100 μg of a second carrier protein (e.g., CRM₁₉₇); and optionally about 0.1 mg to about 0.25 mg of elemental aluminum adjuvant.

In some embodiments, the vaccine or the multivalent pneumococcal conjugate composition (e.g., PCV-22, PCV-23, PCV-24, PCV-25, PCV-26, or PCV-27) may be a single 0.5 ml dose formulated to contain about 2.2 μg of each capsular polysaccharide except serotype 6B, which is present in an amount of about 4.4 μg.

In some embodiments, the vaccine or the multivalent pneumococcal conjugate composition (e.g., PCV-22, PCV-23, PCV-24, PCV-25, PCV-26, or PCV-27) may be a single 0.5 ml dose formulated to contain about 2 μg to about 2.5 μg of each capsular polysaccharide except for up to six capsular polysaccharides selected from the group consisting of serotypes 1, 3, 4, 5, 6B, 9V, 19A, and 19F, each of which is present in an amount of about 4 μg to about 5 μg. In one embodiment, the up to six capsular polysaccharides, present in an amount of about 4 μg to about 5 μg, are selected from the group consisting of serotypes 1, 3, 4, 6B, 9V, 19A, and 19F. In other embodiments, the vaccine or the mixed carrier, multivalent pneumococcal conjugate composition (e.g., PCV-22, PCV-23, PCV-24, PCV-25, PCV-26, or PCV-27) may be a single 0.5 ml dose formulated to contain about 2.2 μg of each capsular polysaccharide except for up to six capsular polysaccharides selected from the group consisting of serotypes 1, 3, 4, 5, 6B, 9V, 19A, and 19F, each of which is present in an amount of about 4.4 μg. In one embodiment, the up to six capsular polysaccharides, present in an amount of about 4.4 μg, are selected from the group consisting of serotypes 1, 3, 4, 6B, 9V, 19A, and 19F.

In some embodiments, the vaccine or the multivalent pneumococcal conjugate composition (e.g., PCV-22, PCV-23, PCV-24, PCV-25, PCV-26, or PCV-27) may be a single 0.5 ml dose formulated to contain about 2 μg to about 2.5 μg of the capsular polysaccharides of serotypes 1, 5, 6A, 7F, 8, 9N, 10A, 11A, 12F, 14, 15A, 15B, 15C, 18C, 22F, 23A, 23B, 23F, 24F, 33F, and/or 35B and about 4 μg to about 5 μg of the capsular polysaccharides of serotypes 3, 4, 6B, 9V, 19A, and/or 19F.

In certain embodiments, the vaccine or the multivalent pneumococcal conjugate composition (e.g., PCV-22, PCV-23, PCV-24, PCV-25, PCV-26, or PCV-27) may be a single 0.5 ml dose formulated to contain about 2 to about 2.5 μg of the capsular polysaccharides of serotypes 1, 4, 5, 6A, 7F, 8, 9V, 9N, 10A, 11A, 12F, 14, 15A, 15B, 15C, 18C, 19A, 19F, 22F, 23A, 23B, 23F, 24F, 33F, and/or 35B and about 4 to about 5 μg of the capsular polysaccharides of serotypes 3 and/or 6B.

In some embodiments, the vaccine or the multivalent pneumococcal conjugate composition (e.g., PCV-22, PCV-23, PCV-24, PCV-25, PCV-26, or PCV-27) may be a single 0.5 ml dose formulated to contain about 2 to about 2.5 μg of the capsular polysaccharides of serotypes 1, 4, 5, 6A, 7F, 8, 9V, 9N, 10A, 11A, 12F, 14, 15A, 15B, 15C, 18C, 19A, 19F, 22F, 23A, 23B, 23F, 24F, 33F, and/or 35B and about 4 to about 5 μg of the capsular polysaccharides of serotype 6B and/or about 8 to about 9 μg of the capsular polysaccharides of serotype 3, and more preferably about 8.8 μg of the capsular polysaccharides of serotype 3.

In certain embodiments, the multivalent pneumococcal conjugate composition (e.g., PCV-22, PCV-23, PCV-24, PCV-25, PCV-26, or PCV-27) or vaccine comprising the same further comprises sodium chloride and sodium succinate buffer as excipients.

In some embodiments, the multivalent pneumococcal conjugate composition (e.g., PCV-22, PCV-23, PCV-24, PCV-25, PCV-26, or PCV-27) may be formulated into a liquid formulation in which each of the pneumococcal capsular polysaccharides from serotypes 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and/or 35B are conjugated to a carrier protein (e.g., CRM₁₉₇). Each 0.5 mL dose may be formulated into a liquid containing: about 2.2 μg of each capsular polysaccharide, except for serotype 6B at about 4.4 μg; about 40 μg to about 100 μg of carrier protein (e.g., CRM₁₉₇); about 0.125 to 0.250 mg of elemental aluminum (about 0.5 to about 1.2 mg aluminum phosphate) as an adjuvant; and sodium chloride and sodium succinate buffer as excipients.

In some embodiments, the mixed carrier, multivalent pneumococcal conjugate composition (e.g., PCV-22, PCV-23, PCV-24, PCV-25, PCV-26, or PCV-27) comprises two or more carrier proteins (mixed carrier). For example, in certain embodiments, at least two serotypes are conjugated to a first carrier protein (e.g., tetanus toxoid) and the remaining serotypes are conjugated to a second carrier protein (e.g., CRM₁₉₇). In certain embodiments, the two capsular polysaccharides that are conjugated to tetanus toxoid are selected from the group consisting of serotypes 1, 3, and 5. In certain embodiments, the two capsular polysaccharides that are conjugated to tetanus toxoid are selected from the group consisting of serotypes 1, 3, 5, 15B, and 22F. It may also be possible to conjugate one or more of serotypes 4, 6A, 6B, 7F, 8, 9N, 9V, 10A, 11A, 12F, 14, 15A, 15C, 18C, 19A, 19F, 23A, 23B, 23F, 24F, 33F, and/or 35B to tetanus toxoid instead of or in addition to the serotypes selected from serotypes 1, 3, 5, 15B, and 22F. Other serotypes of interest may be conjugated to tetanus toxoid.

In some embodiments, the mixed carrier, multivalent pneumococcal conjugate composition (e.g., PCV-22, PCV-23, PCV-24, PCV-25, PCV-26, or PCV-27) may be formulated into a liquid formulation in which each of the pneumococcal capsular polysaccharides of serotypes 1 and 3 is conjugated to TT and the capsular polysaccharides from serotypes 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and/or 35B are conjugated to CRM₁₉₇. Each 0.5 mL dose may be formulated into a liquid containing: about 2.2 μg of each capsular polysaccharide, except for serotype 6B at about 4.4 μg; about 2 μg to about 25 μg of TT carrier protein (only for the serotypes 1 and 3) and about 40 μg to about 100 μg of CRM₁₉₇ carrier protein; about 0.125 to 0.250 mg of elemental aluminum (about 0.5 to about 1.2 mg aluminum phosphate) as an adjuvant; and sodium chloride and sodium succinate buffer as excipients.

In some embodiments, the mixed carrier, multivalent pneumococcal conjugate composition may be formulated into a liquid formulation in which each of the pneumococcal capsular polysaccharides of serotypes 1 and 5 is conjugated to TT and the capsular polysaccharides from serotypes 3, 4, 6A, 6B, 7F, 8, 9N, 9V, 10A, 11A, 12F, 14, 15A, 15B, 15C, 18C, 19A, 19F, 22F, 23A, 23B, 23F, 24F, 33F, and/or 35B are conjugated to CRM₁₉₇. In one embodiment, each 0.5 mL dose may be formulated into a liquid containing: about 2.2 μg of each capsular polysaccharide, except for serotype 6B at about 4.4 μg and serotype 3 at about 2.2-8.8 μg; about 2 μg to about 25 μg of TT carrier protein (only for the serotypes 1 and 5) and about 40 μg to about 100 μg of CRM₁₉₇ carrier protein; about 0.125 to 0.250 mg of elemental aluminum (about 0.5 to 1.2 mg aluminum phosphate) adjuvant; and sodium chloride and sodium succinate buffer as excipients. In certain embodiments, serotype 3 is present at about 2.2 μg. In other embodiments, serotype 3 is present at about 4.4 μg. In other embodiments, serotype 3 is present at about 8.8 μg. In yet another embodiment, each 0.5 mL dose may be formulated into a liquid containing: about 2.2 μg of each capsular polysaccharide, except for up to six capsular polysaccharides selected from the group consisting of serotype 1, 3, 4, 5, 6B, 9V, 19A, and 19F at about 4.4 μg; about 2 μg to about 25 μg of TT carrier protein (only for the serotypes 1 and 5) and about 40 μg to about 100 μg of CRM₁₉₇ carrier protein; about 0.125 mg to 0.250 mg of elemental aluminum (0.5 mg to 1.2 mg aluminum phosphate) adjuvant; and sodium chloride and sodium succinate buffer as excipients. In one embodiment, the up to six capsular polysaccharides at about 4.4 μg are selected from the group consisting of serotype 1, 3, 4, 6B, 9V, 19A, and 19F. In another embodiment, each 0.5 mL dose may be formulated into a liquid containing: about 2.2 μg of each capsular polysaccharide, except for serotypes 3, 4, 6B, 9V, 19A, and 19F at about 4.4 μg; about 2 μg to about 25 μg of TT carrier protein (only for the serotypes 1 and 5) and about 40 μg to about 100 μg of CRM₁₉₇ carrier protein; about 0.125 mg to 0.250 mg of elemental aluminum (0.5 mg to 1.2 mg aluminum phosphate) adjuvant; and sodium chloride and sodium succinate buffer as excipients. In another embodiment, each 0.5 mL dose may be formulated into a liquid containing: about 2.2 μg of each capsular polysaccharide, except for serotypes 3 and 4 at about 4.4 μg; about 2 μg to about 25 μg of TT carrier protein (only for the serotypes 1 and 5) and about 40 μg to about 100 μg of CRM₁₉₇ carrier protein; about 0.125 mg to 0.250 mg of elemental aluminum (0.5 mg to 1.2 mg aluminum phosphate) adjuvant; and sodium chloride and sodium succinate buffer as excipients.

In some embodiments, the mixed carrier, multivalent pneumococcal conjugate composition may be formulated into a liquid formulation in which each of the pneumococcal capsular polysaccharides of serotypes 3 and 5 is conjugated to TT and the capsular polysaccharides from serotypes 1, 4, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B are conjugated to CRM₁₉₇. Each 0.5 mL dose may be formulated into a liquid containing: about 2.2 μg of each capsular polysaccharide, except for 6B at about 4.4 μg; about 2 μg to about 25 μg of TT carrier protein (only for the serotypes 3 and 5) and about 40 μg to about 100 μg of CRM₁₉₇ carrier protein; about 0.125 to 0.250 mg of elemental aluminum (about 0.5 to 1.2 mg aluminum phosphate) adjuvant; and sodium chloride and sodium succinate buffer as excipients.

In some embodiments, the mixed carrier, multivalent pneumococcal conjugate composition may be formulated into a liquid formulation in which at least two of the pneumococcal capsular polysaccharides of serotypes 1, 3, and 5 and both serotypes 15B and 22F are conjugated to tetanus toxoid, and the capsular polysaccharides from the remaining serotypes are conjugated to CRM₁₉₇.

In some embodiments, the mixed carrier, multivalent pneumococcal conjugate composition may be formulated into a liquid formulation in which each of the pneumococcal capsular polysaccharides of serotypes 1, 5, 15B and 22F are conjugated to tetanus toxoid, and the capsular polysaccharides from serotypes 3, 4, 6A, 6B, 7F, 8, 9N, 9V, 10A, 11A, 12F, 14, 15A, 15C, 18C, 19A, 19F, 23A, 23B, 23F, 24F, 33F, and 35B are conjugated to CRM₁₉₇. Each 0.5 mL dose may be formulated into a liquid containing: about 2.2 μg of each capsular polysaccharide, except for 6B at about 4.4 μg; about 2 μg to about 25 μg of TT carrier protein (only for the serotypes 3 and 5) and about 40 μg to about 100 μg of CRM₁₉₇ carrier protein; about 0.125 to 0.250 mg of elemental aluminum (about 0.5 to 1.2 mg aluminum phosphate) adjuvant; and sodium chloride and sodium succinate buffer as excipients.

In some embodiments, the mixed carrier, multivalent pneumococcal conjugate composition may be formulated into a liquid formulation in which each of the pneumococcal capsular polysaccharides of serotypes 1, 3, 15B and 22F are conjugated to tetanus toxoid, and the capsular polysaccharides from serotypes 4, 5, 6A, 6B, 7F, 8, 9N, 9V, 10A, 11A, 12F, 14, 15A, 15C, 18C, 19A, 19F, 23A, 23B, 23F, 24F, 33F, and 35B are conjugated to CRM₁₉₇. Each 0.5 mL dose may be formulated into a liquid containing: about 2.2 μg of each capsular polysaccharide, except for 6B at about 4.4 μg; about 2 μg to about 25 μg of TT carrier protein (only for the serotypes 3 and 5) and about 40 μg to about 100 μg of CRM₁₉₇ carrier protein; about 0.125 to 0.250 mg of elemental aluminum (about 0.5 to 1.2 mg aluminum phosphate) adjuvant; and sodium chloride and sodium succinate buffer as excipients.

In some embodiments, the mixed carrier, multivalent pneumococcal conjugate composition may be formulated into a liquid formulation in which each of the pneumococcal capsular polysaccharides of serotypes 3, 5, 15B and 22F are conjugated to tetanus toxoid, and the capsular polysaccharides from serotypes 1, 4, 6A, 6B, 7F, 8, 9N, 9V, 10A, 11A, 12F, 14, 15A, 15C, 18C, 19A, 19F, 23A, 23B, 23F, 24F, 33F, and 35B are conjugated to CRM₁₉₇. Each 0.5 mL dose may be formulated into a liquid containing: about 2.2 μg of each capsular polysaccharide, except for 6B at about 4.4 μg; about 2 μg to about 25 μg of TT carrier protein (only for the serotypes 3 and 5) and about 40 μg to about 100 μg of CRM₁₉₇ carrier protein; about 0.125 to 0.250 mg of elemental aluminum (about 0.5 to 1.2 mg aluminum phosphate) adjuvant; and sodium chloride and sodium succinate buffer as excipients.

In some embodiments, the mixed carrier, multivalent pneumococcal conjugate composition may be formulated into a liquid formulation in which each of the pneumococcal capsular polysaccharides of serotypes 1 and 5 is conjugated to TT.

In some embodiments, the mixed carrier, multivalent pneumococcal conjugate composition may be formulated into a liquid formulation in which each of the pneumococcal capsular polysaccharides of serotypes 3 and 5 is conjugated to TT.

In some embodiments, the mixed carrier, multivalent pneumococcal conjugate composition may be formulated into a liquid formulation in which each of the pneumococcal capsular polysaccharides of serotypes 1 and 3 is conjugated to TT.

In some embodiments, the mixed carrier, multivalent pneumococcal conjugate composition may be formulated into a liquid formulation in which each of the pneumococcal capsular polysaccharides of serotypes 1, 5, 15B, and 22F is conjugated to TT.

In some embodiments, the mixed carrier, multivalent pneumococcal conjugate composition may be formulated into a liquid formulation in which each of the pneumococcal capsular polysaccharides of serotypes 3, 5, 15B, and 22F is conjugated to TT.

In some embodiments, the mixed carrier, multivalent pneumococcal conjugate composition may be formulated into a liquid formulation in which each of the pneumococcal capsular polysaccharides of serotypes 1, 3, 15B, and 22F is conjugated to TT.

In some embodiments, the liquid formulation may be filled into a single dose syringe without a preservative. After shaking, the liquid formulation becomes a vaccine that is a homogeneous, white suspension ready for intramuscular administration.

The multivalent pneumococcal conjugate composition (e.g., PCV-22, PCV-23, PCV-24, PCV-25, PCV-26, or PCV-27) can be administered in a single injection or as part of an immunization series. For example, the multivalent pneumococcal conjugate composition (e.g., PCV-22, PCV-23, PCV-24, PCV-25, PCV-26, or PCV-27) can be administered 2, 3, 4, or more times at appropriately spaced intervals, such as, a 1, 2, 3, 4, 5, or 6 month interval or a combination thereof. In some embodiments, the multivalent pneumococcal conjugate composition (e.g., PCV-22, PCV-23, PCV-24, PCV-25, PCV-26, or PCV-27) is administered to an infant 4 times within the first 15 months of birth, including, for example, at about 2, 3, 4, and 12-15 months of age; at about 3, 4, 5, and 12-15 months of age; or at about 2, 4, 6, and 12-15 months of age. This first dose can be administered as early as 6 weeks of age. In another embodiment, the multivalent pneumococcal conjugate composition (e.g., PCV-22, PCV-23, PCV-24, PCV-25, PCV-26, or PCV-27) is administered to an infant 3 times within the first 15 months of birth, including, for example, at about 2, 4, and 11-12 months.

The multivalent pneumococcal conjugate composition (e.g., PCV-22, PCV-23, PCV-24, PCV-25, PCV-26, or PCV-27) may also include one or more proteins from Streptococcus pneumoniae. Examples of Streptococcus pneumoniae proteins suitable for inclusion include those identified in International Patent Application WO02/083855, as well as those described in International Patent Application WO02/053761.

The multivalent pneumococcal conjugate composition (e.g., PCV-22, PCV-23, PCV-24, PCV-25, PCV-26, or PCV-27) can be administered to a subject via one or more administration routes known to one of ordinary skill in the art such as a parenteral, transdermal, or transmucosal, intranasal, intramuscular, intraperitoneal, intracutaneous, intravenous, or subcutaneous route and be formulated accordingly. The multivalent pneumococcal conjugate composition (e.g., PCV-22, PCV-23, PCV-24, PCV-25, PCV-26, or PCV-27) can be formulated to be compatible with its intended route of administration.

In some embodiments, the multivalent pneumococcal conjugate composition (e.g., PCV-22, PCV-23, PCV-24, PCV-25, PCV-26, or PCV-27) can be administered as a liquid formulation by intramuscular, intraperitoneal, subcutaneous, intravenous, intraarterial, or transdermal injection or respiratory mucosal injection. The multivalent pneumococcal conjugate compositions (e.g., PCV-22, PCV-23, PCV-24, PCV-25, PCV-26, or PCV-27) can be formulated in liquid form or in a lyophilized form. In some embodiments, injectable compositions are prepared in conventional forms, either as liquid solutions or suspensions, solid forms suitable for solution or suspension in liquid prior to injection, or as emulsions. In some embodiments, injection solutions and suspensions are prepared from sterile powders or granules. General considerations in the formulation and manufacture of pharmaceutical agents for administration by these routes may be found, for example, in Remington's Pharmaceutical Sciences, 19^{th} ed., Mack Publishing Co., Easton, Pa., 1995; incorporated herein by reference. At present the oral or nasal spray or aerosol route (e.g., by inhalation) are most commonly used to deliver therapeutic agents directly to the lungs and respiratory system. In some embodiments, the multivalent pneumococcal conjugate composition (e.g., PCV-22, PCV-23, PCV-24, PCV-25, PCV-26, or PCV-27) is administered using a device that delivers a metered dosage of composition. Suitable devices for use in delivering intradermal pharmaceutical compositions described herein include short needle devices such as those described in U.S. Pat. Nos. 4,886,499, 5,190,521, 5,328,483, 5,527,288, 4,270,537, 5,015,235, 5,141,496, 5,417,662 (all of which are incorporated herein by reference). Intradermal compositions may also be administered by devices which limit the effective penetration length of a needle into the skin, such as those described in WO1999/34850, incorporated herein by reference, and functional equivalents thereof. Also suitable are jet injection devices which deliver liquid vaccines to the dermis via a liquid jet injector or via a needle which pierces the stratum corneum and produces a jet which reaches the dermis. Jet injection devices are described for example in U.S. Pat. Nos. 5,480,381, 5,599,302, 5,334,144, 5,993,412, 5,649,912, 5,569,189, 5,704,911, 5,383,851, 5,893,397, 5,466,220, 5,339,163, 5,312,335, 5,503,627, 5,064,413, 5,520,639, 4,596,556, 4,790,824, 4,941,880, 4,940,460, WO1997/37705, and WO1997/13537 (all of which are incorporated herein by reference). Also suitable are ballistic powder/particle delivery devices which use compressed gas to accelerate vaccine in powder form through the outer layers of the skin to the dermis. Additionally, conventional syringes may be used in the classical Mantoux method of intradermal administration.

Preparations for parenteral administration include sterile aqueous or nonaqueous solutions, suspensions, and emulsions. Examples of non-aqueous solvents are propylene glycol, polyethylene glycol, oils such as olive oil, and injectable organic esters such as ethyl oleate. Examples of oil include vegetable or animal oil, peanut oil, soybean oil, olive oil, sunflower oil, liver oil, synthetic oil such as marine oil, and lipids obtained from milk or eggs. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The multivalent pneumococcal conjugate composition (e.g., PCV-22, PCV-23, PCV-24, PCV-25, PCV-26, or PCV-27) can be formulated in the form of a unit dose vial, multiple dose vial, or pre-filled syringe. A pharmaceutically acceptable carrier for a liquid formulation includes aqueous or nonaqueous solvent, suspension, emulsion, or oil. The composition may be isotonic, hypertonic, or hypotonic. However, it is desirable that the composition for infusion or injection is basically isotonic. Thus, isotonicity or hypertonicity may be advantageous for storage of the composition. When the composition is hypertonic, the composition can be diluted to isotonicity before administration. A tonicity agent may be ionic tonicity agent such as salt or non-ionic tonicity agent such as carbohydrate. The ionic tonicity agent includes, but is not limited to, sodium chloride, calcium chloride, potassium chloride, and magnesium chloride. The nonionic tonicity agent includes, but is not limited to, sorbitol and glycerol. Preferably, at least one pharmaceutically acceptable buffer is included. For example, when the composition is an infusion or injection, it is preferable to be formulated in a buffer with a buffering capacity at pH 4 to pH 10, such as pH 5 to pH 9, or, pH 6 to pH 8. The buffer may be selected from those suitable for United States Pharmacopeia (USP). For example, the buffer can be selected from the group consisting of a monobasic acid, such as acetic acid, benzoic acid, gluconic acid, glyceric acid, and lactic acid; a dibasic acid, such as aconitic acid, adipic acid, ascorbic acid, carbonic acid, glutamic acid, malic acid, succinic acid, and tartaric acid; a polybasic acid such as citric acid and phosphoric acid; and a base such as ammonia, diethanolamine, glycine, triethanolamine, and TRIS.

The multivalent pneumococcal conjugate composition (e.g., PCV-22, PCV-23, PCV-24, PCV-25, PCV-26, or PCV-27) may comprise a surface active agent. Examples of the surface active agent include, but are not limited to, polyoxyethylene sorbitan ester (generally referred to as Tweens), in particular, polysorbate 20 and polysorbate 80; copolymers (such as DOWFAX) of ethylene oxide (EO), propylene oxide (PO), butylenes oxide (BO); octoxynols with different repeats of ethoxy(oxy-1,2-ethanediyl) group, in particular, octoxynol-9 (Triton-100); ethylphenoxypolyethoxyethanol (IGEPAL CA-630/NP-40); phospholipid such as lecithin; nonylphenol ethoxylate such as TERGITOL NP series; lauryl, cetyl, stearyl, oleyl alcohol-derived polyoxyethylene fatty ether (Brij surfactant), in particular, triethyleneglycol monolauryl ether (Brij 30); sorbitan ether known as SPAN, in particular, sorbitan trioleate (Span 85) and sorbitan monolaurate.

Mixtures of surface active agents such as Tween 80/Span 85 can be used. A combination of polyoxyethylene sorbitan ester such as Tween 80 and octoxynol such as Triton X-100 is also suitable. A combination of Laureth 9 and Tween and/or octoxynol is also advantageous. Preferably, the amount of polyoxyethylene sorbitan ester (such as Tween 80) included may be 0.01% to 1% (w/v), 0.01% to 0.1% (w/v), 0.01% to 0.05% (w/v), or about 0.02%; the amount of octylphenoxy polyoxyethanol or nonylphenoxy polyoxyethanol (such as Triton X-100) included may be 0.001% to 0.1% (w/v), in particular 0.005% to 0.02%; and the amount of polyoxyethylene ether (such as Laureth 9) included may be 0.1% to 20% (w/v), possibly 0.1% to 10%, in particular 0.1% to 1% or about 0.5%.

In some embodiments, the multivalent pneumococcal conjugate composition (e.g., PCV-22, PCV-23, PCV-24, PCV-25, PCV-26, or PCV-27) may be delivered via a release control system. For example, intravenous infusion, transdermal patch, liposome, or other routes can be used for administration. In one aspect, macromolecules such as microsphere or implant can be used.

The above disclosure generally describes the present invention. A more complete understanding can be obtained by reference to the following specific examples. These examples are described solely for the purpose of illustration and are not intended to limit the scope of the invention.

### EXAMPLES

### Example 1. Preparation of S. pneumoniae Capsular Polysaccharides

Cultivation of S. pneumoniae and purification of capsular polysaccharides were conducted as known to one of skill in the art. S. pneumoniae serotypes were obtained from the American Type Culture Collection (ATCC) (serotype 1: ATCC No. 6301; serotype 3: ATCC No. 6303; serotype 4: ATCC No. 6304; serotype 5: ATCC No. 6305; serotype 6A: ATCC No. 6306; serotype 6B: ATCC No. 6326; serotype 7F: ATCC No. 10351; serotype 9N: ATCC No. 6309; serotype 9V: ATCC No. 10368; serotype 14: ATCC No. 6314; serotype 18C: ATCC No. 10356; serotype 19A: ATCC No. 10357; serotype 19F: ATCC No. 6319; serotype 23B: ATCC No. 10364; serotype 23F: ATCC No. 6323). For serotypes 8, 10A, 11A, 12F, 15A, 15B, 15C, 22F, 23A, 23B, 24F, 33F, and 35B internal strains or strains obtained from other sources were used, but any publically available strain can be used. S. pneumoniae were characterized by capsules and motility, Gram-positive, lancet-shaped diplococcus, and alpha hemolysis in a blood agar medium. Serotypes were identified by Quelling test using specific anti-sera (U.S. Pat. No. 5,847,112).

Preparation of Cell Banks

Several generations of seed stocks were generated in order to expand the strains and remove components of animal origin (generations F1, F2, and F3). Two additional generations of seed stocks were produced. The first additional generation was cultured from an F3 vial, and the subsequent generation was cultured from a vial of the first additional generation. Seed vials were stored frozen (below −70° C.) with synthetic glycerol as a cryopreservative. For cell bank preparation, all cultures were grown in a soy-based medium. Prior to freezing, cells were concentrated by centrifugation, spent medium was removed, and cell pellets were re-suspended in a fresh medium containing a cryopreservative (such as synthetic glycerol).

Culturing and Harvesting

Cultures from the working cell bank were inoculated into seed bottles containing a soy-based medium and cultured. After the target optical density (absorbance) was reached, the seed bottle was used to inoculate a fermentor containing the soy-based medium. The culturing was terminated when an optical density value started to be maintained constant. After terminating the culturing, sodium deoxycholate was added to the culture to lyse the cells. The resulting fermentor contents were cooled, and protein precipitation was induced. Then, the mixture was centrifuged to remove precipitated proteins and cell debris.

Purification

The solution obtained from the centrifugation was filtered through a depth filter to remove the proteins and cell debris that had not precipitated in the centrifugation. The filtrate was concentrated on a 100 kDa MW membrane and the concentrate was diafiltered with 10 volumes of a 25 mM sodium phosphate buffer (pH 7.2) to obtain a sample. The sample was filtered to collect a supernatant from which polysaccharides were precipitated and filtered. The filtrate was concentrated on a 30 kDa membrane, and the concentrate was diafiltered using about 10 volumes of triple distilled water. After performing the diafiltration, the remaining solution was filtered through a 0.2 μm filter. An in-process control test was performed on the filtrate (appearance, remaining proteins, remaining nucleic acids, endotoxins, molecular weights, and the total amount of polysaccharides). The concentrate was sterile filtered and stored at −20° C.

### Example 2. Preparation of Conjugate of S. pneumoniae Capsular Polysaccharide and Carrier Protein (Serotypes 1, 3, 4, 5, 6A, 6B, 7F, 8, 9N, 9V, 10A, 11A, 12F, 14, 15B, 18C, 19A, 19F, 22F, 23F, and 33F)

Polysaccharides of different serotypes were activated following different pathways and then conjugated to a carrier protein, CRM₁₉₇ or TT. Specifically, a multivalent pneumococcal polysaccharide-protein conjugate comprising capsular polysaccharides from serotypes 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B was prepared by conjugating each of the capsular polysaccharides for serotypes 3, 4, 6A, 6B, 7F, 8, 9N, 9V, 10A, 11A, 12F, 14, 15B, 18C, 19A, 19F, 22F, 23F, and 33F to CRM₁₉₇ and by conjugating each of the capsular polysaccharides of the serotypes 1 and 5 to TT, as disclosed below. The conjugation of serotypes 15A, 15C, 23A, 23B, 24F, and 35B to CRM₁₉₇ is described in Examples 3-8. Another multivalent pneumococcal polysaccharide-protein conjugate comprising capsular polysaccharides from serotypes 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B was prepared by conjugating each of the capsular polysaccharides of serotypes 3, 4, 6A, 6B, 7F, 8, 9N, 9V, 10A, 11A, 12F, 14, 18C, 19A, 19F, 23F, and 33F to CRM₁₉₇ and by conjugating each of the capsular polysaccharides of the serotypes 1, 5, 15B, and 22F to TT, as disclosed below.

In place of or in addition to serotypes 1 or 5, it is also contemplated that serotype 3 may be conjugated to TT, as disclosed in WO2019/152925. Depending on the size of the native serotype the activation process may include reduction of the size of each capsular polysaccharide to the target molecular weight, chemical activation, and buffer exchange via ultrafiltration.

Polysaccharides of different serotypes were activated following different pathways and then conjugated to a carrier protein, CRM₁₉₇ or TT. Specifically, conjugates were prepared by conjugating each of the capsular polysaccharides of all serotypes, except 15B and 22F, to CRM₁₉₇ and by conjugating each of the capsular polysaccharides of the serotypes 1, 3, 5, 15B and 22F to TT. Depending on the size of the native serotype the activation process may include reduction of the size of each capsular polysaccharide to the target molecular weight, chemical activation, and buffer exchange via ultrafiltration. The conjugates were purified using ultrafiltration and finally filtered through 0.2 μm filter. The process parameters such as pH, temperature, concentration, and time were as follows.

(1) Activation Process

Step 1: Hydrolysis

Reductive amination is a known method for conjugating polymers in which an amide bond is formed between a primary amine (—NH₂) group of a protein and an aldehyde of a saccharide. Aldehyde groups are added to the pneumococcal capsular polysaccharide to promote conjugation to the carrier protein. A vicinal diol structure of a monosaccharide can be oxidized by sodium periodate (NaIO₄) to form aldehyde groups. The capsular polysaccharides from serotypes 1, 3, 4, 6A, 8, 11A, 12F, 14, 15B, 18C, 22F, and 33F were pre-treated as follows.

In the case of the serotype 1, sodium hydroxide (at a final base concentration of 0.05 M) was added to a solution of the capsular polysaccharide, and the solution was incubated at 50±2° C. The solution was then cooled to a temperature in a range of about 21° C. to about 25° C., and hydrochloric acid was added thereto to a final pH of 6.0±0.1, thereby stopping hydrolysis.

In the case of the serotype 3, 8, 11A, and 15B, hydrochloric acid (at a final acid concentration of 0.01 M) was added to a solution of the capsular polysaccharide, and the solution was incubated at 60±2° C. The solution was then cooled to a temperature in a range of about 21° C. to about 25° C., and 0.1M sodium phosphate was added thereto to a final pH of 6.0±0.1, thereby stopping hydrolysis.

In the case of the serotype 4, hydrochloric acid (at a final acid concentration of 0.1 M) was added to a solution of the capsular polysaccharide, and the solution was incubated at 45±2° C. The solution was then cooled to a temperature in a range of about 21° C. to about 25° C., and 1M sodium phosphate was added thereto to a final pH of 6.0±0.1, thereby stopping hydrolysis.

In the case of the serotype 6A, glacial acetic acid (at a final acid concentration of 0.1 M) was added to a solution of the capsular polysaccharide, and the solution was incubated at 60±2° C. The solution was then cooled to a temperature in a range of about 21° C. to about 25° C., and 1M sodium hydroxide was added thereto to a final pH of 6.0±0.1, thereby stopping hydrolysis.

In the case of the serotype 12F, hydrochloric acid (at a final acid concentration of 0.01 M) was added to a solution of the capsular polysaccharide, and the solution was incubated at 70±2° C. The solution was then cooled to a temperature in a range of about 21° C. to about 25° C., and 0.1M sodium phosphate was added thereto to a final pH of the solution of 6.0±0.1, thereby stopping hydrolysis.

In the case of the serotypes 14 and 18C, glacial acetic acid (at a final acid concentration of 0.2 M) was added to a solution of the capsular polysaccharide, and the solution was incubated at 94±2° C. The solution was then cooled to a temperature in a range of about 21° C. to about 25° C., and 1M sodium phosphate was added thereto so that a final pH of the solution was 6.0±0.1, thereby stopping hydrolysis.

In the case of the serotypes 22F and 33F, hydrochloric acid (at a final acid concentration of 0.01 M) was added to a solution of the capsular polysaccharide, and the solution was incubated at 60±2° C. The solution was then cooled to a temperature in a range of about 21° C. to about 25° C., and 0.1M sodium phosphate was added thereto to a final pH of 6.0±0.1, thereby stopping hydrolysis.

Each of the obtained capsular polysaccharides was diluted in water for injection (WFI), sodium acetate, and sodium phosphate to a final concentration between about 1.0 mg/mL and about 2.0 mg/mL.

Step 2: Periodate Reaction

The sodium periodate molar equivalent for each pneumococcal saccharide activation was determined based on repeating unit molar mass. With thorough mixing, the oxidation reaction was allowed to proceed for 16 to 20 hours at 21° C. to 25° C. for all serotypes except for 1, 7F, and 19F, for which the temperature was 10° C. or less. To help maintain consistent and stable production of conjugates, a range of degree of oxidation (Do) levels for each serotype is targeted during the conjugation process. A preferred, targeted range for the Do levels for each serotype is shown in Table 1 and Table 2.

TABLE 1
Range of Do for all serotypes to be conjugated to CRM₁₉₇

| Serotype | Range of Do |
|---|---|
| Serotype 1 | 4 to 10 |
| Serotype 3 | 2 to 8 |
| Serotype 4 | 1 to 5 |
| Serotype 6A | 5 to 15 |
| Serotype 6B | 7 to 13 |
| Serotype 7F | 2 to 8 |
| Serotype 8 | 1 to 17 |
| Serotype 9N | 5 to 10 |
| Serotype 9V | 4 to 9 |
| Serotype 10A | 1 to 12 |
| Serotype 11A | 1 to 15 |
| Serotype 12F | 1 to 9 |
| Serotype 14 | 6 to 13 |
| Serotype 18C | 6 to 14 |
| Serotype 19A | 20 to 40 |
| Serotype 19F | 20 to 40 |
| Serotype 23F | 6 to 14 |
| Serotype 33F | 1 to 15 |

TABLE 2
Range of Do for serotypes 1, 3, 5,
15B and 22F to be conjugated to TT

| Serotype | Range of Do |
|---|---|
| Serotype 1 (1-TT) | 1 to 15 |
| Serotype 3 (3-TT) | 2 to 14 |
| Serotype 5 (5-TT) | 1 to 15 |
| Serotype 15B (15B-TT) | 1 to 15 |
| Serotype 22F (22F-TT) | 20 to 50 |

Step 3: Ultrafiltration

The oxidized saccharide was concentrated and diafiltered with WFI on a 100 kDa MWCO ultrafilter (30 kDa ultrafilter for serotype 1 and 5 kDa ultrafilter for serotype 18C). Diafiltration was conducted using 0.9% sodium chloride solution for serotype 1, 0.01 M sodium acetate buffer (pH 4.5) for serotype 7F and 23F, and 0.01 M sodium phosphate buffer (pH 6.0) for serotype 19F. The permeate was discarded, and the retentate was filtered through a 0.2 μm filter.

Step 4: Lyophilization

For capsular polysaccharides of serotypes 3, 4, 5, 8, 9N, 9V, 10A, 14, and 33F that are to be conjugated to a carrier protein by using an aqueous solvent, mixed solution of polysaccharides and carrier protein was prepared without adding further sucrose, lyophilized, and then stored at −25° C.±5° C.

For capsular polysaccharides of serotypes 1 and 18C that are to be conjugated to a carrier protein by using an aqueous solvent, polysaccharides and carrier protein were independently prepared, without adding further sucrose, lyophilized, and then stored at −25° C.±5° C.

For capsular polysaccharides of serotypes 6A, 6B, 7F, 15B-TT, 19A, 19F, 22F-TT and 23F that are to be conjugated to a carrier protein by using a DMSO solvent, a predetermined amount of sucrose to reach a final sucrose concentration of 5%±3% (w/v) was added to the activated saccharides, and the samples were independently prepared, lyophilized, and then stored at −25° C.±5° C.

For capsular polysaccharide of serotype 11A, a predetermined amount of sucrose to reach a final sucrose concentration of 20%±5% (w/v) was added to the activated saccharide, and the polysaccharides and carrier protein were independently prepared, lyophilized, and then stored at −25° C.±5° C.

For capsular polysaccharide of serotype 12F, a predetermined amount of sucrose to reach a final sucrose concentration of 10%±5% (w/v) was added to the activated saccharide, and the polysaccharides and carrier protein were independently prepared, lyophilized, and then stored at −25° C.±5° C.

(2) Conjugation Process

Aqueous conjugation was conducted for serotypes 1, 3, 4, 5, 8, 9N, 9V, 10A, 14, 18C, and 33F, and DMSO conjugation was conducted for serotypes 6A, 6B, 7F, 11A, 12F, 15B-TT, 19A, 19F, 22F-TT and 23F. Each of the capsular polysaccharides was conjugated to a carrier protein at a ratio of 0.2 to 2:1.

Step 1: Dissolution

Aqueous Conjugation

For serotypes 1, 3, 4, 5, 8, 9N, 9V, 10A, 14, 18C, and 33F, the lyophilized sample was thawed and equilibrated at room temperature. The lyophilized sample was reconstituted to a reaction concentration by using a sodium phosphate buffer solution at 23±2° C. at a ratio set for each serotype.

Dimethyl Sulfoxide (DMSO) Conjugation

For serotypes 6A, 6B, 7F, 11A, 12F, 15B-TT, 19A, 19F, 22F-TT, and 23F, the lyophilized sample was thawed, equilibrated at room temperature, and reconstituted in DMSO.

Step 2: Conjugation Reaction

Aqueous Conjugation

For serotypes 3-TT, 4, 5-TT, 8, 9N, 9V, 10A, 14, 18C, and 33F, the conjugation reaction was initiated by adding the sodium cyanoborohydride solution (100 mg/mL) to 1.0 to 1.4 moles sodium cyanoborohydride per mole of saccharide. However, for serotypes 1, 1-TT and 3, the reaction was initiated by adding the sodium cyanoborohydride solution to 0.5 moles sodium cyanoborohydride per mole of saccharide.

The reaction mixture was incubated at 23° C. to 37° C. for 44 to 106 hours. The reaction temperature and time were adjusted by serotype. The temperature was then reduced to 23±2° C. and sodium chloride 0.9% was added to the reactor. Sodium borohydride solution (100 mg/mL) was added to achieve 1.8 to 2.2 molar equivalents of sodium borohydride per mole of saccharide. The mixture was incubated at 23±2° C. for 3 to 6 hours. This procedure reduced any unreacted aldehydes present on the saccharides. Then, the mixture was diluted with sodium chloride 0.9% and the diluted conjugation mixture was filtered using a 0.8 or 0.45 μm pre-filter.

DMSO Conjugation

For capsular polysaccharides of serotypes 6A, 6B, 7F, 11A, 12F, 15B-TT, 19A, 19F, 22F-TT and 23F, the conjugation reaction was initiated by adding the sodium cyanoborohydride solution (100 mg/mL) to a ratio of 0.8 to 1.2 molar equivalents of sodium cyanoborohydride per one mole of activated saccharide. WFI was added to the reaction mixture to a target concentration of 1% (v/v), and the mixture was incubated for 12 to 26 hours at 23±2° C. 100 mg/mL of a sodium borohydride solution (typical 1.8 to 2.2 molar equivalents sodium borohydride per mole activated saccharide) and WFI (target 5% v/v) were added to the reaction and the mixture was incubated for 3 to 6 hours at 23±2° C. This procedure reduced any unreacted aldehydes present on the saccharides. Then, the reaction mixture was diluted with sodium chloride 0.9%, and the diluted conjugation mixture was filtered using a 0.8 or 0.45 μm pre-filter.

Step 3: Ultrafiltration

The diluted conjugate mixture was concentrated and diafiltered on a 100 kDa MWCO ultrafiltration filter or a 300 kDa MWCO ultrafiltration filter with a minimum of 15 volumes of 0.9% sodium chloride or buffer. Also, the composition and pH of the buffer used in the process varied depending on each of the serotypes.

Step 4: Sterile Filtration

The retentate after the ultrafiltration was sterile filtered (0.2 μm), and in-process controls (appearance, free protein, free saccharide, molecular size distribution, sterility, saccharide content, protein content, pH, endotoxin, residual cyanide, residual DMSO, saccharide identity, TT identity, and CRM₁₉₇ identity) were performed on the filtered conjugates. The final concentrate was refrigerated and stored at 2° C. to 8° C.

The conjugation of serotypes 15A, 15C, 23A, 23B, 24F, and 35B to CRM₁₉₇ is described in Examples 3-8.

### Example 3. Preparation of Monoconjugate of Serotype 15A and CRM₁₉₇

A serotype 15A polysaccharide can be purified as discussed above or by reference to the methods described in WO2013/191459 for purifying polysaccharides of other serotypes. Acid hydrolysis was performed by applying acid and heat to the purified serotype 15A polysaccharide as shown in Table 1 and then an activation process was performed. It was observed that the conditions of hydrolysis affected the degree of oxidation (Do) and the molecular weight of the activated polysaccharide, as well as the conjugation results. The activation process and a conjugation process were performed under the same conditions. Sodium periodate was added and the oxidation reaction was carried out at 21 to 25° C. for 16 to 20 hours. The activated polysaccharide and CRM₁₉₇ protein were lyophilized and suspended in DMSO. The activated polysaccharide and protein were mixed at a ratio of 1:1 while the reaction concentration was 1.5 mg/mL based on the polysaccharide content. Cyanoborohydride was added to initiate the conjugation reaction, and the mixture was incubated at 23° C.±2° C. for 20 to 28 hours. The borohydride solution mixture was incubated at 23° C.±2° C. for 3 to 6 hours. Through this process, any unreacted aldehyde present in the saccharide was reduced, followed by concentration and dialysis with an ultrafiltration filter.

TABLE 3
15A conjugation results according to hydrolysis conditions

| Activated | | | 15A-CRM₁₉₇ | | | |
|---|---|---|---|---|---|---|
| polysaccharide | | | conjugate | | | |
| | | Activated | | Free | | |
| Hydrolysis | | PS M.W. | Ratio | PS | MSD | MALLS |
| Condition | Do | (kDa) | (PS/PR) | (%) | (%) | (kDa) |
| — | 13.4 | 340.8 | 0.98 | 76.5 | 86 | 30517 |
| 0.01M HCl, | 11.5 | 304.5 | 1.20 | 79.0 | 93 | 25933 |
| 60° C., | | | | | | |
| 60 min | | | | | | |
| 0.01M HCl, | 10.6 | 373.4 | 1.09 | 76.7 | 94 | 25899 |
| 60° C., | | | | | | |
| 90 min | | | | | | |
| 0.01M HC1. | 10.8 | 350.8 | 0.97 | 73.7 | 88 | 24734 |
| 60° C., | | | | | | |
| 120 min | | | | | | |
| 0.1M HCl. | 16.0 | 197.0 | 1.34 | 76.6 | | 32898 |
| 60° C., | | | | | | |
| 45 min | | | | | | |
| 0.1M HCl, | 7.9 | 116.3 | 0.73 | 11.4 | | 3924 |
| 60° C., | | | | | | |
| 90 min | | | | | | |

The effect of oxidation levels (Do) on the conjugation of serotype 15A and CRM₁₉₇ was assessed. 0.1M HCl was added to the 15A polysaccharide at 60° C. for 90 minutes. The amount of sodium periodate was adjusted and the oxidation reaction was carried out at 21 to 25° C. for 16 to 20 hours. The activated polysaccharide and CRM₁₉₇ protein were lyophilized and suspended in DMSO. The activated polysaccharide and protein were mixed at a ratio of 1:1 while the reaction concentration was 1.5 mg/mL based on the polysaccharide content and conjugation with cyanoborohydride was carried out at as described above when assessing the effect of acid hydrolysis on serotype 15A.

TABLE 4
15A conjugation results according to oxidation levels

| | | 15A-CRM₁₉₇ | | | | |
|---|---|---|---|---|---|---|
| Activated | | conjugate | | | | |
| polysaccharide | | | | | | Conju- |
| | Activated | | Free | | | gation |
| | PS M.W. | Ratio | PS | MSD | MALLS | yield |
| Do | (kDa) | (PS/PR) | (%) | (%) | (kDa) | (%) |
| 19.6 | 139.7 | — | — | — | 888 | 9.8 |
| 17.7 | 158.6 | — | — | — | 1753 | 3.9 |
| 7.9 | 116.3 | 0.73 | 11.4 | | 3924 | 43.8 |
| 4.4 | 75.7 | 0.77 | 1.3 | | 1411 | 47.1 |
| 3.9 | 69.3 | 0.71 | 1.1 | | 1217 | 43.7 |

The effect of the reaction ratio of polysaccharide to protein on conjugation was also assessed. The activated 15A polysaccharide and CRM₁₉₇ protein were lyophilized and suspended in DMSO. The activated polysaccharide and protein were mixed at a ratio described in Table 5 and the reaction concentration was 1.0 mg/mL based on the polysaccharide content and conjugation with cyanoborohydride was carried out at described above when assessing the effect of acid hydrolysis on serotype 15A.

TABLE 5
Conjugation results according to polysaccharide to protein ratio

| | | | 15A-CRM₁₉₇ | | | | |
|---|---|---|---|---|---|---|---|
| Activated | | Reaction | conjugate | | | | |
| polysaccharide | | ratio | | | | | Conju- |
| | Activated | Reaction | | Free | | | gation |
| | PS M.W. | ratio | Ratio | PS | MSD | MALLS | yield |
| Do | (kDa) | (PR:PS) | (PS/PR) | (%) | (%) | (kDa) | (%) |
| 4.7 | 67.9 | 2:1 | 0.44 | 3.2 | 69 | 3631 | 59.0 |
| | | 1.75:1 | 0.46 | 3.2 | 71 | 3616 | 50.8 |
| | | 1.5:1 | 0.52 | 1.9 | 71 | 3834 | 39.0 |
| | | 1.25:1 | 0.66 | 3.1 | 61 | 2027 | 50.5 |
| | | 1:1 | 0.62 | 3.3 | 59 | 1707 | 40.8 |

### Example 4. Preparation of Monoconjugate of Serotype 15C and CRM₁₉₇

A serotype 15C polysaccharide can be purified as discussed above or by reference to the methods described in WO2013/191459 for purifying polysaccharides of other serotypes. The amount of sodium periodate added to the 15C polysaccharide was adjusted and the oxidation reaction was carried out at 21 to 25° C. for 16 to 20 hours. The activated polysaccharide and CRM₁₉₇ protein were lyophilized and suspended in phosphate buffer. The activated polysaccharide and protein were mixed at a ratio of 1:1 while the reaction concentration was 15 mg/mL based on the polysaccharide content. Cyanoborohydride was added to initiate the conjugation reaction, and the mixture was incubated at 37° C.±2° C. for 44 to 52 hours. The borohydride solution mixture was incubated at 23° C.±2° C. for 3 to 6 hours. Through this process, any unreacted aldehyde present in the saccharide was reduced, followed by concentration and dialysis with an ultrafiltration filter.

TABLE 6
15C conjugation results according to oxidation
levels using phosphate buffer

| | | 15C-CRM₁₉₇ | | | | |
|---|---|---|---|---|---|---|
| Activated | | conjugate | | | | |
| polysaccharide | | | | | | Conju- |
| | Activated | | Free | | | gation |
| | PS M.W. | Ratio | PS | MSD | MALLS | yield |
| Do | (kDa) | (PS/PR) | (%) | (%) | (kDa) | (%) |
| 35.1 | 657.6 | 5.49 | 31.2 | 85 | 1506 | 67.4 |
| 31.1 | 549.9 | 5.71 | 37.8 | 77 | 1268 | 73.2 |
| 18.4 | 678.6 | 3.24 | 12.5 | 89 | 2885 | 67.0 |
| 16.3 | 525.3 | 3.29 | 17.8 | 76 | 1877 | 68.3 |
| 8.9 | 768.5 | 1.83 | 4.6 | 91 | 2767 | 54.8 |
| 8.1 | 510.1 | 2.26 | 7.8 | 78 | 4833 | 63.4 |
| 5.5 | 755.7 | 1.50 | 1.8 | 85 | 3899 | 17.4 |
| 4.5 | 472.4 | 1.67 | 7.2 | 75 | 5618 | 39.2 |
| 2.3 | 471.0 | 1.36 | 3.2 | 62 | 2610 | 13.8 |

The effect of oxidation levels (Do) on conjugation of serotype 15C and CRM₁₉₇ using DMSO was assessed. The amount of sodium periodate added to the 15C polysaccharide was adjusted and the oxidation reaction was carried out at 21 to 25° C. for 16 to 20 hours. The activated polysaccharide and CRM₁₉₇ protein were lyophilized and suspended in DMSO. The activated polysaccharide and protein were mixed at a ratio of 1:1 while the reaction concentration was 1.5 mg/mL based on the polysaccharide content. Cyanoborohydride was added to initiate the conjugation reaction, and the mixture was incubated at 23° C.±2° C. for 20 to 28 hours. The borohydride solution mixture was incubated at 23° C.±2° C. for 3 to 6 hours. Through this process, any unreacted aldehyde present in the saccharide was reduced, followed by concentration and dialysis with an ultrafiltration filter.

TABLE 7
15C conjugation results according to oxidation levels using DMSO

| | | 15C-CRM₁₉₇ | | | | |
|---|---|---|---|---|---|---|
| Activated | | conjugate | | | | |
| polysaccharide | | | | | | Conju- |
| | Activated | | Free | | | gation |
| | PS M.W. | Ratio | PS | MSD | MALLS | yield |
| Do | (kDa) | (PS/PR) | (%) | (%) | (kDa) | (%) |
| 31.1 | 549.9 | 1.28 | 19.8 | 91 | 13992 | 61.6 |
| 16.3 | 525.3 | 1.16 | 4.7 | 86 | 13733 | 57.2 |
| 8.1 | 510.1 | 1.11 | 2.7 | 77 | 6248 | 18.0 |
| 4.5 | 472.4 | 1.04 | 2.5 | 78 | 9364 | 31.0 |
| 2.3 | 471.0 | 0.87 | 2.7 | 80 | 8105 | 41.7 |

### Example 5. Preparation of Monoconjugate of Serotype 23A and CRM₁₉₇

A serotype 23A polysaccharide can be purified as discussed above or by reference to the methods described in WO2013/191459 for purifying polysaccharides of other serotypes. To assess the effect of degree of oxidation (Do) on conjugation, the amount of sodium periodate was adjusted and the oxidation reaction was carried out at 21 to 25° C. for 16 to 20 hours. The activated polysaccharide and CRM₁₉₇ protein were lyophilized and suspended in DMSO. The activated polysaccharide and protein were mixed at a ratio of 1:1 while the reaction concentration was 1 mg/mL based on the polysaccharide content. Alternatively, the activated polysaccharide and protein were mixed at a ratio described in Table 8, while the reaction concentration was 1.5 mg/mL based on the polysaccharide content. Cyanoborohydride was added to initiate the conjugation reaction, and the mixture was incubated at 23° C.±2° C. for 20 to 28 hours. The borohydride solution mixture was incubated at 23° C.±2° C. for 3 to 6 hours. Through this process, any unreacted aldehyde present in the saccharide was reduced, followed by concentration and dialysis with an ultrafiltration filter. The effect of varying Do levels on conjugation are shown in Table 8.

TABLE 8
23A conjugation results according to oxidation levels

| | | 23A-CRM₁₉₇ | | | | |
|---|---|---|---|---|---|---|
| Activated | | conjugate | | | | |
| polysaccharide | | | | | | Conju- |
| | Activated | | Free | | | gation |
| | PS M.W. | Ratio | PS | MSD | MALLS | yield |
| Do | (kDa) | (PS/PR) | (%) | (%) | (kDa) | (%) |
| 23.9 | 429.0 | 1.17 | 32.2 | 98 | 6561 | 27.9 |
| 17.3 | 472.6 | 1.11 | 19.7 | 96 | 4982 | 69.9 |
| 10.9 | 640.0 | 0.97 | 3.2 | 92 | 4335 | 62.2 |
| 9.7 | 486.8 | 1.08 | 21.5 | 89 | 2917 | 55.9 |
| 8.8 | 565.0 | 1.22 | 33.7 | 91 | 5081 | 45.3 |
| 6.7 | 489.0 | 1.01 | 19.2 | 87 | 2635 | 71.1 |

The effect of the reaction ratio of polysaccharide to protein on conjugation are shown in Table 9.

TABLE 9
23A conjugation results according
to polysaccharide to protein ratio

| | | | 23A-CRM₁₉₇ | | | | |
|---|---|---|---|---|---|---|---|
| Activated | | Reaction | conjugate | | | | |
| polysaccharide | | ratio | | | | | Conju- |
| | Activated | Reaction | | Free | | | gation |
| | PS M.W. | ratio | Ratio | PS | MSD | MALLS | yield |
| Do | (kDa) | (PR:PS) | (PS/PR) | (%) | (%) | (kDa) | (%) |
| 10.9 | 640.0 | 2:1 | 0.49 | 0 | 91 | 11065 | 36.0 |
| | | 1.75:1 | 0.59 | 0.2 | 91 | 8345 | 52.9 |
| | | 1.5:1 | 0.66 | 1.3 | 90 | 4963 | 26.7 |
| | | 1.25:1 | 0.80 | 1.1 | 91 | 5524 | 52.0 |
| | | 1:1 | 0.97 | 3.2 | 92 | 4335 | 62.2 |

The effect of the reaction concentration on conjugation between serotype 23A and CRM₁₉₇ was assessed. Purified serotype 23A polysaccharide was activated with sodium periodate as discussed above. The activated polysaccharide and CRM₁₉₇ protein were lyophilized and suspended in DMSO. The activated polysaccharide and protein were mixed at a ratio of 1:1, while the reaction concentration was as described in Table 10 based on the polysaccharide content and conjugation with cyanoborohydride was carried out at described above when assessing the effect of Do on serotype 23A.

TABLE 10
23A conjugation results according to
concentrations of conjugate reaction

| | | Reaction | 23A-CRM₁₉₇ | | | | |
|---|---|---|---|---|---|---|---|
| Activated | | concen- | conjugate | | | | |
| polysaccharide | | tration | | | | | Conju- |
| | Activated | Reaction | | Free | | | gation |
| | PS M.W. | Conc. | Ratio | PS | MSD | MALLS | yield |
| Do | (kDa) | (mg/ml) | (PS/PR) | (%) | (%) | (kDa) | (%) |
| 8.8 | 565.0 | 1.0 | 1.22 | 33.7 | 91 | 5081 | 45.3 |
| | | 1.5 | 1.27 | 20.5 | 93 | 6612 | 29.4 |
| | | 2.0 | 1.37 | 35.1 | 96 | 4373 | 19.3 |
| | | 2.5 | 2.63 | 21.3 | — | 2223 | 4.1 |
| | | 3.0 | — | — | — | 2471 | 2.3 |

The effect of acid hydrolysis on conjugation between serotype 23A and CRM₁₉₇ was assessed. Acid hydrolysis was performed by applying acid and heat to the purified serotype 23A polysaccharide as shown in Table 11 and then an activation process was performed. The activation process and a conjugation process were performed under the same conditions. Sodium periodate was added and the oxidation reaction was carried out at 21 to 25° C. for 16 to 20 hours. The activated polysaccharide and CRM₁₉₇ protein were lyophilized and suspended in phosphate buffer. The activated polysaccharide and protein were mixed at a ratio of 1:1 while the reaction concentration was 1.5 mg/mL based on the polysaccharide content. Cyanoborohydride was added to initiate the conjugation reaction, and the mixture was incubated at 37° C.±2° C. for 44 to 52 hours. The borohydride solution mixture was incubated at 23° C.±2° C. for 3 to 6 hours. Through this process, any unreacted aldehyde present in the saccharide was reduced, followed by concentration and dialysis with an ultrafiltration filter.

TABLE 11
Preparation of 23A glycoconjugates after
acid hydrolysis of polysaccharides

| | | | 23A-CRM₁₉₇ | | | | |
|---|---|---|---|---|---|---|---|
| Activated | | | conjugate | | | | |
| polysaccharide | | | | | | | Conju- |
| | | Activated | | Free | | | gation |
| Hydrolysis | | PS M.W. | Ratio | PS | MSD | MALLS | yield |
| condition | Do | (kDa) | (PS/PR) | (%) | (%) | (kDa) | (%) |
| X | 10.9 | 639.3 | 1.21 | 22.0 | 89 | 5981 | 19.6 |
| 0.1M HCl, | 9.4 | 595.4 | 1.17 | 17.0 | 91 | 5844 | 13.2 |
| 60° C., | | | | | | | |
| 30 min | | | | | | | |
| 0.1M HCl, | 9.1 | 571.1 | 1.21 | 10.9 | 82 | 3360 | 10.5 |
| 60° C., | | | | | | | |
| 60 min | | | | | | | |
| 0.1M HCl, | 9.0 | 535.3 | 1.07 | 9.9 | 83 | 2911 | 10.3 |
| 60° C., | | | | | | | |
| 90 min | | | | | | | |
| 0.1M HCl, | 9.3 | 507.9 | 1.26 | 5.2 | 64 | 1846 | 5.6 |
| 60° C., | | | | | | | |
| 120 min | | | | | | | |

The effect of using a phosphate buffer on conjugation between serotype 23A and CRM₁₉₇ was assessed. The amount of sodium periodate was adjusted in order to activate serotype 23A and the oxidation reaction was carried out at 21 to 25° C. for 16 to 20 hours. The activated serotype 23A polysaccharide and CRM₁₉₇ protein were lyophilized and suspended in phosphate buffer. The activated polysaccharide and protein were mixed at a ratio of 1:1 while the reaction concentration was 15 mg/mL based on the polysaccharide content. Cyanoborohydride was added to initiate the conjugation reaction, and the mixture was incubated at 23° C.±2° C. for 20 to 28 hours. The borohydride solution mixture was incubated at 23° C.±2° C. for 3 to 6 hours. Through this process, any unreacted aldehyde present in the saccharide was reduced, followed by concentration and dialysis with an ultrafiltration filter.

TABLE 12
Preparation of 23A glycoconjugates using phosphate buffer

| | | 23A-CRM₁₉₇ | | | | |
|---|---|---|---|---|---|---|
| Activated | | conjugate | | | | |
| polysaccharide | | | | | | Conju- |
| | Activated | | Free | | | gation |
| | PS M.W. | Ratio | PS | MSD | MALLS | yield |
| Do | (kDa) | (PS/PR) | (%) | (%) | (kDa) | (%) |
| 18.0 | 630.1 | 2.98 | 73.1 | 89 | 2790 | 58.9 |
| 9.5 | 633.1 | 2.41 | 61.5 | 88 | 1901 | 35.9 |
| 6.6 | 627.5 | 1.74 | 33.0 | 77 | 2067 | 21.8 |
| 5.1 | 613.4 | 1.50 | 27.9 | 80 | 2794 | 20.9 |
| 4.5 | 608.0 | 1.07 | 19.4 | 81 | 6182 | 37.5 |

### Example 6. Preparation of Monoconjugate of Serotype 23B and CRM₁₉₇

A serotype 23B polysaccharide can be purified as discussed above or by reference to the methods described in WO2013/191459 for purifying polysaccharides of other serotypes. To assess the effect of the degree of oxidation (Do) on conjugation, the amount of sodium periodate was adjusted in order to activate serotype 23B and the oxidation reaction was carried out at 21 to 25° C. for 16 to 20 hours. The activated polysaccharide and CRM₁₉₇ protein were lyophilized and suspended in DMSO. The activated polysaccharide and protein were mixed at a ratio of 1:1 while the reaction concentration was 1.5 mg/mL based on the polysaccharide content. Alternatively, the amount of sodium periodate was held constant and the activated polysaccharide and protein were mixed at a ratio described in Table 13, while the reaction concentration was 1.5 mg/mL based on the polysaccharide content. Cyanoborohydride was added to initiate the conjugation reaction, and the mixture was incubated at 23° C.±2° C. for 20 to 28 hours. The borohydride solution mixture was incubated at 23° C.±2° C. for 3 to 6 hours. Through this process, any unreacted aldehyde present in the saccharide was reduced, followed by concentration and dialysis with an ultrafiltration filter. The effect of varying Do levels on conjugation are shown in Table 13.

TABLE 13
23B conjugation results according to oxidation levels

| | | 23B-CRM₁₉₇ | | | | |
|---|---|---|---|---|---|---|
| Activated | | conjugate | | | | |
| polysaccharide | | | | | | Conju- |
| | Activated | | Free | | | gation |
| | PS M.W. | Ratio | PS | MSD | MALLS | yield |
| Do | (kDa) | (PS/PR) | (%) | (%) | (kDa) | (%) |
| 23.3 | 590.8 | 1.33 | 87.1 | 98 | 34676 | 67.7 |
| 14.2 | 583.7 | 1.25 | 74.4 | 99 | 20774 | 63.0 |
| 7.4 | 674.5 | 0.74 | 36.7 | 95 | 4891 | 47.0 |
| 6.6 | 547.3 | 0.95 | 65.4 | 97 | 9609 | 57.7 |
| 5.7 | 631.4 | 0.84 | 46.9 | 81 | 5149 | 52.9 |
| 5.5 | 549.7 | 1.12 | 85.6 | 92 | 8839 | 70.2 |
| 5.4 | 635.8 | 1.03 | 50.9 | 90 | 5647 | 63.9 |
| 2.8 | 391.4 | 0.64 | 50.3 | 77 | 5213 | 31.1 |
| 2.3 | 221.6 | 0.27 | 26.3 | 56 | 3878 | 21.2 |
| 0.9 | 267.5 | 0.23 | 39.9 | 49 | 2588 | 7.2 |

The effect of the reaction ratio of polysaccharide to protein on conjugation are shown in Table 14.

TABLE 14
23B conjugation results according to polysaccharide to protein ratio

| | | | 23B-CRM₁₉₇ | | | | |
|---|---|---|---|---|---|---|---|
| Activated | | Reaction | conjugate | | | | |
| polysaccharide | | Ratio | | | | | Conju- |
| | Activated | Reaction | | Free | | | gation |
| | PS M.W. | ratio | Ratio | PS | MSD | MALLS | yield |
| Do | (kDa) | (PR:PS) | (PS/PR) | (%) | (%) | (kDa) | (%) |
| 2.3 | 221.6 | 2:1 | 0.21 | 15.2 | 69 | 6720 | 25.0 |
| | | 1.75:1 | 0.22 | 20.5 | 71 | 6408 | 22.6 |
| | | 1.5:1 | 0.23 | 19.6 | 68 | 4694 | 16.7 |
| | | 1.25:1 | 0.31 | 34.0 | 58 | 2448 | 21.2 |
| | | 1:1 | 0.27 | 26.3 | 56 | 3878 | 12.0 |

### Example 7. Preparation of Monoconjugate of Serotype 24F and CRM₁₉₇

A serotype 24F polysaccharide can be purified as discussed above or by reference to the methods described in WO2013/191459 for purifying polysaccharides of other serotypes. Purified serotype 24F polysaccharide was subjected to an acid hydrolysis or a microfluidizer, followed by adding sodium periodate to serotype 24F polysaccharide, and the oxidation reaction was carried out at 21 to 25° C. for 16 to 20 hours. The activated polysaccharide and CRM₁₉₇ protein were lyophilized and suspended in phosphate buffer. The activated polysaccharide and protein were mixed at a ratio of 1:1 while the reaction concentration was 10 mg/mL based on the polysaccharide content. Cyanoborohydride was added to initiate the conjugation reaction, and the mixture was incubated at 37° C.±2° C. for 44 to 52 hours. The borohydride solution mixture was incubated at 23° C.±2° C. for 3 to 6 hours. Through this process, any unreacted aldehyde present in the saccharide was reduced, followed by concentration and dialysis with an ultrafiltration filter. The molar equivalent of cyanoborohydride and borohydride was as described in Table 15. As cyanoborohydride was added to the activated 24F polysaccharide and CRM₁₉₇ protein instead of capping reagent (borohydride), the quality of the conjugate improves, as indicated by increasing molecular weight of the conjugate. Adding an excess amount of capping reagent (borohydride) had a negative effect on the 24F-CRM₁₉₇ conjugate, as indicated by decreasing molecular weight of the conjugate.

TABLE 15
Conjugation results according to amount
of reducing agent (PO₄ buffer)

| Activated | | 24F-CRM₁₉₇ | | | | |
|---|---|---|---|---|---|---|
| polysaccharide | | conjugate | | | | |
| | Activated | | | | Free | |
| | PS M.W. | NaCNBH3 | NaBH4 | Ratio | PS | MALLS |
| Do | (kDa) | Meq | Meq | (PS/PR) | (%) | (kDa) |
| 98.5 | 241.8 | 1.2 | — | 3.72 | 35.5 | 3295 |
| | | 1.2/0.5 | — | 3.91 | 32.4 | 3413 |
| | | 1.2/1.0 | — | 3.81 | 29.5 | 4562 |
| | | 1.2 | 0.1 | 3.39 | 30.2 | 3096 |
| | | 1.2 | 0.5 | 3.33 | 23.0 | 2615 |
| | | 1.2 | 1.0 | 3.51 | 27.7 | 1807 |

### Example 8: Preparation of Monoconjugate of Serotype 35B and CRM₁₉₇

A serotype 35B polysaccharide can be purified as discussed above or by reference to the methods described in WO2013/191459 for purifying polysaccharides of other serotypes. Purified serotype 35B polysaccharide was diluted in DW (distilled water) to a final concentration of 1.0 mg/mL to 2.0 mg/mL.

Periodic Acid Reaction. To assess the effect of the degree of oxidation (Do) on conjugation, the amount of sodium periodate was adjusted in order to activate serotype 35B and the oxidation reaction was carried out at 21 to 25° C. for 16 to 20 hours. A molar equivalent of 0.007 to 0.15 sodium periodate relative to the polysaccharide content was used.

Ultrafiltration. The activated serotype 35B polysaccharides were concentrated and diafiltered with DW using a 30 kDa MWCO ultrafiltration filter. The permeate was discarded and the residue was filtered through a 0.22 μm filter.

Lyophilization. A specific amount of sucrose calculated to reach a 5%±3% sucrose concentration was added to the activated serotype 35B polysaccharide. Concentrated saccharide and CRM₁₉₇ carrier protein were each filled in vials and lyophilized. Alternatively, the activated serotype 35B polysaccharide and the carrier protein were mixed and filled into a glass bottle and lyophilized.

Dissolving. The lyophilized activated serotype 35B saccharide and lyophilized CRM₁₉₇ carrier protein were equilibrated at room temperature. The activated serotype 35B saccharide was resuspended in phosphate buffer at a concentration of 12.5 g/L to 17.5 g/L saccharide. The pH of the phosphate buffer for the conjugation reaction was adjusted to pH 6.0 to pH 7.2. At this time, the carrier protein was used at a concentration of 6.25 g/L to about 35 g/L (PR:PS weight ratio corresponds to 1:0.5 to 2).

Conjugation Reaction. The conjugation reaction was initiated by adding sodium cyanoborohydride solution (100 mg/mL) at a ratio of 1.0 to 1.4 molar equivalents per 1 mole of the activated saccharide. The mixture was incubated at 37±2° C. for 44 to 52 hours. A 100 mg/mL of sodium borohydride solution (usually 1.8 to 2.2 molar equivalents of sodium borohydride per 1 mole of activated saccharide) was added to the reaction material and the mixture was incubated at 23° C.±2° C. for 3 to 6 hours. Through this process, any unreacted aldehyde present in the saccharide was reduced, followed by concentration and dialysis with an ultrafiltration filter. The reaction mixture was then diluted with 0.9% sodium chloride and the diluted conjugate mixture was filtered through a 0.45 μm filter.

Ultrafiltration. The diluted conjugate mixture was concentrated and diafiltered using 100 kDa MWCO ultrafiltration filter with at least 20 volumes of 0.9% sodium chloride solution or buffer. The permeate was discarded.

Sterile Filtration. The residue after 100 kDa MWCO diafiltration was filtered through a 0.22 μm filter. In-process control (saccharide content, free protein, free saccharide, and residual cyanide) was performed on the filtered product 35B-CRM₁₉₇ conjugate. In-process control was performed on the filtered residue to determine whether additional concentration, diafiltration and/or dilution was needed. If necessary, the filtered conjugate was diluted with 0.9% sodium chloride so that the final concentration was less than 0.55 g/L. At this stage, tests for saccharide content, protein content and saccharide:protein ratios were performed. The conjugate was filtered (0.22 μm filter) and free tests (appearance, free proteins, free saccharides, endotoxins, molecular sizing, residual cyanide, saccharide identity and CRM₁₉₇ identity) were performed. The serotype 35B glycoconjugate comprises at least 0.2 mM of acetate per mM of 35B polysaccharide. The final conjugate concentrate was refrigerated at 2 to 8° C. Analysis results for some representative preparation examples of the serotype 35B glycoconjugate are shown in Table 16 below.

TABLE 16
Conjugation results according to oxidation levels (PO₄ buffer)

| | | 35B-CRM₁₉₇ | | | | |
|---|---|---|---|---|---|---|
| Activation conditions | | conjugate | | | | |
| and results | | | | | | Conju- |
| Molecular | | | | Free | | gation |
| weight | Degree of | buffer | Ratio | saccharide | MALLS | yield |
| (kDa) | activation | pH | (PS/PR) | (%) | (kDa) | (%) |
| 103.6 | 26.8 | 7.2 | 1.51 | 26.8 | 1162 | 48.4 |
| 85.7 | 26.2 | 7.2 | 1.74 | 26.0 | 413 | 75.2 |
| 97.8 | 26.2 | 7.2 | 2.04 | 29.6 | 323 | 30.9 |
| 97.8 | 26.2 | 6.0 | 1.27 | 19.1 | 3153 | 40.9 |
| 104.5 | 25.0 | 7.2 | 1.80 | 15.8 | 449 | 46.2 |
| 101.8 | 23.3 | 7.2 | 1.55 | 37.5 | 433 | 23.6 |
| 99.0 | 23.0 | 6.0 | 1.76 | 28.0 | 2211 | 43.7 |
| 93.8 | 21.3 | 7.2 | 2.20 | 32.8 | 568 | 21.6 |
| 80.6 | 20.5 | 6.0 | 1.53 | 35.4 | 2621 | 23.4 |
| 84.8 | 20.5 | 6.0 | 1.93 | 26.1 | 4301 | 31.6 |
| 63.1 | 20.1 | 7.2 | 1.31 | 23.1 | 370 | 43.7 |
| 63.1 | 20.1 | 6.0 | 1.08 | 17.5 | 1966 | 42.0 |
| 81.0 | 19.5 | 6.0 | 1.37 | 23.4 | 1043 | 25.2 |
| 58.9 | 18.5 | 7.2 | 1.74 | 14.6 | 1211 | 66.6 |
| 70.5 | 17.7 | 7.2 | 1.56 | 18.1 | 786 | 37.4 |
| 56.6 | 17.1 | 6.0 | 1.42 | 17.3 | 1044 | 51.3 |
| 61.9 | 16.5 | 7.2 | 1.39 | 12.5 | 722 | 60.4 |
| 48.6 | 14.6 | 7.2 | 1.21 | 18.5 | 584 | 47.0 |
| 45.4 | 12.9 | 7.2 | 1.12 | 12.6 | 716 | 60.9 |
| 41.8 | 12.8 | 7.2 | 1.51 | 14.6 | 1031 | 49.0 |
| 46.1 | 12.7 | 6.0 | 1.21 | 14.8 | 1539 | 49.1 |
| 45.0 | 12.6 | 6.0 | 1.17 | 11.2 | 1219 | 54.8 |
| 45.0 | 12.6 | 7.2 | 1.36 | 12.4 | 676 | 42.4 |
| 36.5 | 12.2 | 6.0 | 0.96 | 7.7 | 1155 | 66.0 |
| 34.8 | 12.0 | 7.2 | 0.90 | 5.0 | 643 | 23.3 |
| 47.0 | 11.9 | 7.2 | 1.33 | 19.1 | 826 | 47.5 |
| 37.1 | 9.7 | 6.0 | 1.10 | 11.7 | 1202 | 41.8 |
| 35.9 | 7.1 | 6.0 | 0.96 | 12.6 | 1079 | 33.4 |

As seen in Table 16, the methods described herein for making a serotype 35B glycoconjugate showed good conjugation yields and allows for the preparation of conjugates with low free saccharide % and good stability.

### Example 9: Serotype Specific IgG Concentration Measurements

A serotype 15A-CRM₁₉₇ monoconjugate prepared in Example 3, a serotype 15C-CRM₁₉₇ monoconjugate prepared in Example 4, a serotype 23A-CRM₁₉₇ monoconjugate prepared in Example 5, a serotype 23B-CRV1197 monoconjugate prepared in Example 6, a serotype 24F-CRM₁₉₇ monoconjugate prepared in Example 7, and a serotype 35B-CRM₁₉₇ monoconjugate prepared in Example 8 were tested for the ability to induce an immunogenic response in rabbits. Immunogenicity assessment was performed by antigen-specific ELISA for serum IgG concentrations and by opsonophagocytic assay (OPA) for antibody functionality. New Zealand White rabbits were immunized intramuscularly at week 0 and week 2 with the human dose (2.2 μg of polysaccharide). Sera were sampled every 2 weeks post immunization.

Capsular polysaccharides (PnPs) for each of serotypes 15A, 15C, 23A, 23B, 24F, and 35B were coated on a 96-well plate at 0.5 μg/well to 1 μg/well. An equivalent amount of serum was sampled from each subject and was pooled by group. The plates were washed with washing buffer and incubated with blocking buffer for 1 hour at 37° C. The serum pool was serially diluted by 2.5 times with an antibody dilution buffer comprising Tween 20 and pneumococcal cell-wall polysaccharide (CWPS) obtained from Statens Serum Institut (5 μg/mL) and then reacted at room temperature for 30 minutes. The plate was washed 5 times with a washing buffer and then pre-adsorbed and diluted serum 50 μl was added to the coated well plate, followed by incubation at room temperature for 2 hours to 18 hours. The well plate was washed in the same way and then goat anti-Rabbit IgG-alkaline phosphatase conjugates were added to each well, followed by incubation at room temperature for 2 hours. Plates were washed as described above and 1 mg/mL p-nitrophenylamine buffer as substrate was added to each well and then reacted at room temperature for 2 hours. The reaction was quenched by adding 50 μl of 3 M NaOH and absorbances at 405 nm and 690 nm were measured. The results are shown in Table 17.

TABLE 17
IgG concentration (U/mL) at 2 weeks
after secondary immunization

| Serotype | Pre-1 | Post-1 | Pre-2 | Post-2 |
|---|---|---|---|---|
| 15A | 312.5 | 5637.2 | 312.5 | 5703.2 |
| 15C | 130 | 84369.0 | — | — |
| 23A | 130 | 367.9 | — | — |
| 23B | 141.5 | 30775.9 | 152.1 | 185474.0 |
| 24F | 309.0 | 3200.1 | 476.1 | 4529.5 |

For serotype 35B, the IgG concentration was measured for different groups of glycoconjugates based on the pH of the conjugation reaction and the molecular weight of the 35B glycoconjugate, as set forth in Table 18.

TABLE 18
IgG concentration at 2 weeks after secondary immunization

| | 35B glycoconjugate | | | |
|---|---|---|---|---|
| | pH of | Molecular | | |
| | conjugation | weight | IgG Concentration U/mL (95% CI) | |
| Group | reaction | (kDa) | Pretest | Post |
| 1 | 7.2 | 1162 | 130 | 19333 |
| | | | (130-130) | (7330-50991) |
| 2 | 7.2 | 323 | 130 | 10309 |
| | | | (130-130) | (5271-20160) |
| 3 | 6.0 | 3153 | 130 | 12317 |
| | | | (130-130) | (6718-22584) |

* CI: confidence interval
The significance of group 1, 2 and 3: P = 0.384

Functional Immunogenicity Test (MOPA) for Monovalent Conjugates

Antibody functions were evaluated by testing serum in a MOPA assay. S. pneumoniae MOPA strain stored at −70° C. or lower was diluted to the corresponding final dilution fold so that a concentration of each strain was about 50,000 CFU/mL. An equivalent amount of serum was sampled from each subject, pooled by group and 2-fold serially diluted so that 20 μl of serum remained in a U-bottom plate. After diluting the sample, 10 μl of the strain prepared for each serotype was mixed with the diluted sample, and the mixture was allowed to react at room temperature for 30 minutes so that S. pneumoniae and the antibody were well mixed. A mixture of pre-differentiated HL-60 cells and complement was added and reacted in a CO₂ incubator (37° C.) for 45 minutes. The temperature was reduced to stop phagocytosis and 10 μl of the reaction solution was spotted onto a THY agar plate pre-dried for 30 to 60 minutes, and then allowed to be absorbed onto the plate for 20 minutes until drying. A 25 mg/mL TTC stock solution was added to a prepared overlay agar, and an antibody appropriate for the corresponding strain was added thereto. The mixture was thoroughly mixed, and then about 25 mL of the mixture was added onto the plate and hardened for about 30 minutes. The completely hardened plate was incubated in a C02 incubator (37° C.) for 12 to 18 hours and then colonies were counted. MOPA titer was expressed as a dilution rate at which 50% killings were observed. The results are shown in Table 19.

TABLE 19
MOPA titers for Monoconjugates at 2
weeks after secondary immunization

| Serotype | Pre-1 | Post-1 | Pre-2 | Post-2 |
|---|---|---|---|---|
| 15A | 5 | 3555 | 10 | 3107 |
| 15C | 2 | 31575.3 | — | — |
| 23A | 2 | 313.7 | — | — |
| 23B | 28 | 18807 | 57 | 7375 |
| 24F | 2 | 260 | 2 | 367 |

For serotype 35B, the MOPA titers were measured for different groups of glycoconjugates based on the pH of the conjugation reaction and the molecular weight of the 35B glycoconjugate, as set forth in Table 20.

TABLE 20
MOPA titers for 35B-CRM₁₉₇ at 2 weeks
after secondary immunization

| | 35B glycoconjugate | | | |
|---|---|---|---|---|
| | pH of | Molecular | | |
| | conjugation | weight | Geometric mean titer (95% CI*) | |
| Group | reaction | (kDa) | Pretest | Post |
| 1 | 7.2 | 1162 | 2 | 16019 |
| | | | | (8621-29766) |
| 2 | 7.2 | 323 | 2 | 11568 |
| | | | | (6434-20798) |
| 3 | 6.0 | 3153 | 2 | 14151 |
| | | | | (9027-22183) |

*CI: confidence interval
The significance of group 1, 2 and 3: P = 0.621

### Example 10: Formulation of 27-Valent Pneumococcal Conjugate Vaccine with Polysaccharides from Serotypes 1 and 5 Conjugated to Tetanus Toxoid

The desired volumes of final bulk concentrates obtained from Examples 2-8 were calculated based on the batch volume and the bulk saccharide concentrations. After the 0.85% sodium chloride (physiological saline), polysorbate 80, and succinate buffer were added to the pre-labeled formulation vessel, bulk concentrates were added. The preparation was then thoroughly mixed and sterile filtered through a 0.2 μm membrane. The formulated bulk was mixed gently during and following the addition of bulk aluminum phosphate. The pH was checked and adjusted if necessary. The formulated bulk product was stored at 2 to 8° C. The following multivalent pneumococcal conjugate vaccine formulation was prepared and named, PCV27-(1/5)-TT.

PCV27(1/5)-TT includes polysaccharide-conjugates prepared by conjugating each polysaccharide of the serotypes 1 and 5 to TT and each polysaccharides of the serotypes 3, 4, 6A, 6B, 7F, 8, 9N, 9V, 10A, 11A, 12F, 14, 15A, 15B, 15C, 18C, 19A, 19F, 22F, 23A, 23B, 23F, 24F, 33F and 35B to CRM₁₉₇.

The PCV27(1/5)-TT in a total dose of 0.5 ml included 2.2 μg of each saccharide, except for serotype 6B at 4.4 μg; about 2 μg to 25 μg of TT (for serotypes 1 and 5) and about 45 μg to 100 μg of CRM₁₉₇; 0.125 mg of elemental aluminum (0.5 mg aluminum phosphate) adjuvant; 4.25 mg of sodium chloride; about 295 μg of a succinate buffer solution; and about 120 μg of polysorbate 80.

### Example 11. Immunogenicity of Multivalent Pneumococcal Conjugate Vaccine (PCV27(1/5)-TT)

The mixed carrier, multivalent pneumococcal vaccine, PCV27(1/5)-TT prepared in Example 10, was tested for the ability to induce an immunogenic response in rabbits. Immunogenicity assessment was performed by antigen-specific ELISA for serum IgG concentrations and by opsonophagocytic assay (OPA) for antibody functionality. New Zealand White rabbits were immunized intramuscularly at week 0 and week 2 with the human dose (2.2 μg of each polysaccharide, except for 6B at 4.4 μg). Sera were sampled every 2 weeks post immunization.

Serotype Specific IgG Concentration Measurement

Capsular polysaccharides (PnPs) for each serotype were coated on a 96-well plate at 0.5 μg/well to 1 μg/well. An equivalent amount of serum was sampled from each subject and was pooled by group. The serum pool was serially diluted by 2.5 times with an antibody dilution buffer comprising Tween 20 and pneumococcal cell-wall polysaccharide (CWPS) obtained from Statens Serum Institut (5 μg/mL) and then reacted at room temperature for 30 minutes. The plate was washed 5 times with a washing buffer and then pre-adsorbed and diluted serum 50 μl was added to the coated well plate, followed by incubation at room temperature for 2 hours to 18 hours. The well plate was washed in the same way and then goat anti-Rabbit IgG-alkaline phosphatase conjugates were added to each well, followed by incubation at room temperature for 2 hours. Plates were washed as described above and 1 mg/mL p-nitrophenylamine buffer as substrate was added to each well and then reacted at room temperature for 2 hours. The reaction was quenched by adding 50 μl of 3 M NaOH and absorbances at 405 nm and 690 nm were measured. The results are shown in Table 21.

TABLE 21
IgG concentration (U/mL) at 2 weeks
after secondary immunization

| | PCV27(1/5)-TT | PCV27(1/5)-TT |
|---|---|---|
| Serotype | Pre | Post |
| 1 | 130.0 | 5929.1 |
| 3 | 130.0 | 3161.6 |
| 4 | 130.0 | 5883.6 |
| 5 | 130.0 | 14645.5 |
| 6A | 130.0 | 5823.0 |
| 6B | 130.0 | 725.1 |
| 7F | 130.0 | 6569.9 |
| 8 | 251.4 | 11743.1 |
| 9N | 130.0 | 20552.6 |
| 9V | 130.0 | 13890.2 |
| 10A | 130.0 | 5400.2 |
| 11A | 130.0 | 5769.0 |
| 12F | 130.0 | 2004.1 |
| 14 | 147.9 | 2631.1 |
| 15A | 130.0 | 1632.9 |
| 15B | 130.0 | 13427.9 |
| 15C | 130.0 | 22777.7 |
| 18C | 130.0 | 12984.3 |
| 19A | 130.0 | 2117.9 |
| 19F | 130.0 | 5464.1 |
| 22F | 130.0 | 11996.1 |
| 23A | 130.0 | 173.7 |
| 23B | 155.4 | 4392.8 |
| 23F | 130.0 | 568.7 |
| 24F | 361.1 | 1141.5 |
| 33F | 130.0 | 11650.0 |
| 35B | 130.0 | 656.6 |

Functional Immunogenicity Test (MOPA)

Antibody functions were evaluated by testing serum in a MOPA assay. S. pneumoniae MOPA strain stored at −70° C. or lower was diluted to the corresponding final dilution fold so that a concentration of each strain was about 50,000 CFU/mL. An equivalent amount of serum was sampled from each subject, pooled by group and 2-fold serially diluted so that 20 μl of serum remained in a U-bottom plate. After diluting the sample, 10 μl of the strain prepared for each serotype was mixed with the diluted sample, and the mixture was allowed to react at room temperature for 30 minutes so that S. pneumoniae and the antibody were well mixed. A mixture of pre-differentiated HL-60 cells and complement was added and reacted in a CO₂ incubator (37° C.) for 45 minutes. The temperature was reduced to stop phagocytosis and 10 μl of the reaction solution was spotted onto an agar plate pre-dried for 30 to 60 minutes, and then allowed to be absorbed onto the plate for 20 minutes until drying. A 25 mg/mL TTC stock solution was added to a prepared overlay agar, and an antibody appropriate for the corresponding strain was added thereto. The mixture was thoroughly mixed, and then about 25 mL of the mixture was added onto the plate and hardened for about 30 minutes. The completely hardened plate was incubated in a CO₂ incubator (37° C.) for 12 to 18 hours and then colonies were counted. MOPA titer was expressed as a dilution rate at which 50% killings were observed. The results are shown in Table 22.

TABLE 22
MOPA titers at 2 weeks after secondary immunization

| | PCV27(1/5)-TT | PCV27(1/5)-TT |
|---|---|---|
| Serotype | Pre | Post |
| 1 | 2 | 237 |
| 3 | 2 | 240 |
| 4 | 2 | 1564 |
| 5 | 2 | 975 |
| 6A | 2 | 981 |
| 6B | 2 | 241 |
| 7F | 2 | 479 |
| 8 | 36 | 901 |
| 9N | 2 | 2108 |
| 9V | 2 | 261 |
| 10A | 2 | 219 |
| 11A | 16 | 730 |
| 12F | 2 | 193 |
| 14 | 22 | 416 |
| 15A | 2 | 3099 |
| 15B | 22 | 501 |
| 15C | 2 | 6743 |
| 18C | 2 | 816 |
| 19A | 2 | 261 |
| 19F | 2 | 571 |
| 22F | 6 | 477 |
| 23A | 2 | 203 |
| 23B | 2 | 565 |
| 23F | 2 | 221 |
| 24F | 2 | 143 |
| 33F | 2 | 312 |
| 35B | 6 | 627 |

### Example 12: Formulation of 27-Valent Pneumococcal Conjugate Vaccine with

Polysaccharides from Serotypes 1, 5, 15B, and 22F Conjugated to Tetanus Toxoid

Monoconjugates were obtained following the general methods described in Examples 2-8. The desired volumes of final bulk concentrates were calculated based on the batch volume and the bulk saccharide concentrations. After the 0.85% sodium chloride (physiological saline), polysorbate 80, and succinate buffer were added to the pre-labeled formulation vessel, bulk concentrates were added. The preparation was then thoroughly mixed and sterile filtered through a 0.2 μm membrane. The formulated bulk was mixed gently during and following the addition of bulk aluminum phosphate. The pH was checked and adjusted if necessary. The formulated bulk product was stored at 2 to 8° C. The following multivalent pneumococcal conjugate vaccine formulation was prepared and named, PCV27-(1/5/15B/22F)-TT.

PCV27(1/5/15B/22F)-TT includes polysaccharide-conjugates prepared by conjugating each polysaccharide of the serotypes 1, 5, 15B, and 22F to TT and each polysaccharides of the serotypes 3, 4, 6A, 6B, 7F, 8, 9N, 9V, 10A, 11A, 12F, 14, 15A, 15C, 18C, 19A, 19F, 23A, 23B, 23F, 24F, 33F and 35B to CRM₁₉₇.

The PCV27(1/5/15B/22F)-TT in a total dose of 0.5 ml included 2.2 μg of each saccharide, except for serotype 6B at 4.4 μg; about 2 μg to 25 μg of TT (for serotypes 1, 5, 15B, 22F) and about 45 μg to 100 μg of CRM₁₉₇; 0.125 mg of elemental aluminum (0.5 mg aluminum phosphate) adjuvant; 4.25 mg of sodium chloride; about 295 μg of a succinate buffer solution; and about 120 μg of polysorbate 80.

### Example 13. Immunogenicity of Multivalent Pneumococcal Conjugate Vaccine, PCV27(1/5/15B/22F)-TT

The mixed carrier, multivalent pneumococcal vaccine, PCV27(1/5/15B/22F)-TT prepared in Example 12, was tested for the ability to induce an immunogenic response in rabbits. Immunogenicity assessment was performed by antigen-specific ELISA for serum IgG concentrations and by opsonophagocytic assay (OPA) for antibody functionality. New Zealand White rabbits were immunized intramuscularly at week 0 and week 2 with the human dose (2.2 μg of each polysaccharide, except for 6B at 4.4 μg). Sera were sampled every 2 weeks post immunization.

Serotype Specific IgG Concentration Measurement

Capsular polysaccharides (PnPs) for each serotype were coated on a 96-well plate at 0.5 μg/well to 1 μg/well. An equivalent amount of serum was sampled from each subject and was pooled by group. The serum pool was serially diluted by 2.5 times with an antibody dilution buffer comprising Tween 20 and pneumococcal cell-wall polysaccharide (CWPS) obtained from Statens Serum Institut (5 μg/mL) and then reacted at room temperature for 30 minutes. The plate was washed 5 times with a washing buffer and then pre-adsorbed and diluted serum 50 μl was added to the coated well plate, followed by incubation at room temperature for 2 hours to 18 hours. The well plate was washed in the same way and then goat anti-Rabbit IgG-alkaline phosphatase conjugates were added to each well, followed by incubation at room temperature for 2 hours. Plates were washed as described above and 1 mg/mL p-nitrophenylamine buffer as substrate was added to each well and then reacted at room temperature for 2 hours. The reaction was quenched by adding 50 μl of 3 M NaOH and absorbances at 405 nm and 690 nm were measured. As a comparative example, the commercially available, 13-valent vaccine (PREVNAR13) was subjected to the same procedure. The results are shown in Table 23.

TABLE 23
IgG concentration (U/mL) at 2 weeks
after secondary immunization

| | |
|---|---|
| Prevnar13 | PCV27(1/5/15B/22F)-TT |

| Serotype | Pre | Post | Pre | Post |
|---|---|---|---|---|
| 1 | 130.0 | 471.2 | 130.0 | 6174.5 |
| 3 | 173.7 | 1052.8 | 130.0 | 4296.6 |
| 4 | 130.0 | 1267.1 | 130.0 | 5237.9 |
| 5 | 130.0 | 1551.7 | 130.0 | 28839.0 |
| 6A | 130.0 | 722.4 | 130.0 | 2538.2 |
| 6B | 130.0 | 252.9 | 130.0 | 671.5 |
| 7F | 130.0 | 5969.8 | 130.0 | 6056.5 |
| 8 | 130.0 | 130.0 | 140.6 | 7108.9 |
| 9N | 169.4 | 225.0 | 211.2 | 25748.1 |
| 9V | 130.0 | 7085.3 | 130.0 | 6228.0 |
| 10A | 130.0 | 130.0 | 130.0 | 3022.1 |
| 11A | 130.0 | 130.0 | 130.0 | 3928.1 |
| 12F | 130.0 | 130.0 | 130.0 | 1393.4 |
| 14 | 130.0 | 777.7 | 107.3 | 1132.9 |
| 15A | 130.0 | 130.0 | 130.0 | 416.8 |
| 15B | 130.0 | 130.0 | 130.0 | 4196.6 |
| 15C | 130.0 | 130.0 | 130.0 | 5882.5 |
| 18C | 143.2 | 2387.1 | 130.0 | 7544.2 |
| 19A | 147.7 | 1643.5 | 130.0 | 899.9 |
| 19F | 130.0 | 6623.0 | 130.0 | 12785.6 |
| 22F | 130.0 | 130.0 | 130.0 | 5492.0 |
| 23A | 130.0 | 158.5 | 130.0 | 840.1 |
| 23B | 203.4 | 2098.3 | 141.6 | 2765.6 |
| 23F | 130.0 | 566.4 | 130.0 | 588.8 |
| 24F | 377.6 | 362.7 | 362.7 | 2351.9 |
| 33F | 130.0 | 130.0 | 130.0 | 3573.9 |
| 35B | 148.0 | 150.5 | 130.0 | 936.6 |

Functional Immunogenicity Test (MOPA)

When the capsular polysaccharides of serotypes 1 and 5 were conjugated to TT, the serotype specific IgG concentration significantly increased compared to that obtained when they were conjugated to CRM₁₉₇. Rabbits immunized with PCV27(1/5/15B/22F)-TT also demonstrated significant increases in IgG concentration against the additional fourteen serotypes not present in PREVNAR13 (i.e., 8, 9N, 10A, 11A, 12F, 15A, 15B, 15C, 22F, 23A, 23B, 24F, 33F and 35B). Serotypes 8 and 9N, in particular, had a greater than 50-fold increase in serum specific IgG concentration relative to PREVNAR13.

Antibody functions were evaluated by testing serum in a MOPA assay. S. pneumoniae MOPA strain stored at −70° C. or lower was diluted to the corresponding final dilution fold so that a concentration of each strain was about 50,000 CFU/mL. An equivalent amount of serum was sampled from each subject, pooled by group and 2-fold serially diluted so that 20 μl of serum remained in a U-bottom plate. After diluting the sample, 10 μl of the strain prepared for each serotype was mixed with the diluted sample, and the mixture was allowed to react at room temperature for 30 minutes so that S. pneumoniae and the antibody were well mixed. A mixture of pre-differentiated HL-60 cells and complement was added and reacted in a C02 incubator (37° C.) for 45 minutes. The temperature was reduced to stop phagocytosis and 10 μl of the reaction solution was spotted onto an agar plate pre-dried for 30 to 60 minutes, and then allowed to be absorbed onto the plate for 20 minutes until drying. A 25 mg/mL TTC stock solution was added to a prepared overlay agar, and an antibody appropriate for the corresponding strain was added thereto. The mixture was thoroughly mixed, and then about 25 mL of the mixture was added onto the plate and hardened for about 30 minutes. The completely hardened plate was incubated in a C02 incubator (37° C.) for 12 to 18 hours and then colonies were counted. MOPA titer was expressed as a dilution rate at which 5000 killings were observed. As a comparative example, the commercially available, 13-valent vaccine (PREVNAR13) was subjected to the same procedure. The results are shown in Table 24.

TABLE 24
MOPA titers at 2 weeks after secondary immunization

| | |
|---|---|
| Prevnar13 | PCV27(1/5/15B/22F)-TT |

| Serotype | Pre | Post | Pre | Post |
|---|---|---|---|---|
| 1 | 2 | 72 | 61 | 527 |
| 3 | 18 | 154 | 131 | 280 |
| 4 | 2 | 91 | 2 | 374 |
| 5 | 2 | 213 | 182 | 1967 |
| 6A | 2 | 253 | 2 | 292 |
| 6B | 2 | 250 | 2 | 591 |
| 7F | 2 | 218 | 2 | 259 |
| 8 | 2 | 2 | 2 | 671 |
| 9N | 2 | 2 | 2 | 1564 |
| 9V | 2 | 289 | 2 | 99 |
| 10A | 2 | 2 | 2 | 92 |
| 11A | 2 | 2 | 2 | 10923 |
| 12F | 2 | 2 | 2 | 140 |
| 14 | 2 | 213 | 2 | 273 |
| 15A | 2 | 80 | 2 | 635 |
| 15B | 10 | 40 | 49 | 199 |
| 15C | 25 | 73 | 56 | 1837 |
| 18C | 2 | 356 | 2 | 940 |
| 19A | 2 | 468 | 2 | 211 |
| 19F | 2 | 240 | 2 | 628 |
| 22F | 2 | 2 | 2 | 639 |
| 23A | 2 | 66 | 2 | 404 |
| 23B | 2 | 426 | 2 | 823 |
| 23F | 2 | 223 | 2 | 222 |
| 24F | 2 | 2 | 2 | 169 |
| 33F | 2 | 2 | 2 | 80 |
| 35B | 2 | 51 | 2 | 237 |

When the serotypes 1 and 5 were conjugated to TT, functional MOPA titers significantly increased compared to MOPA titers obtained when they were conjugated to CRM₁₉₇. Rabbits immunized with PCV27(1/5/15B/22F)-TT also demonstrated significant increases in functional MOPA titers against each of the additional fourteen serotypes that are not present in PREVNAR13 (i.e., 8, 9N, 10A, 1 IA, 12F, 15A, 15B, 15C, 22F, 23A, 23B, 24F, 33F and 35B).

While one or more exemplary embodiments have been described in the specification, it will be understood by those of ordinary skill in the art that various changes in form and details may be made therein without departing from the spirit and scope of the inventive concept as defined by the following claims.

1. A multivalent pneumococcal conjugate composition, comprising 22-27 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae serotypes are selected from 1, 3, 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B.

2. The multivalent pneumococcal conjugate composition of claim 1, comprising 27 different pneumococcal capsular polysaccharide-protein conjugates, wherein each pneumococcal capsular polysaccharide-protein conjugate comprises a protein carrier conjugated to a capsular polysaccharide from a different serotype of Streptococcus pneumoniae, wherein the Streptococcus pneumoniae serotypes are selected from 1, 3, 4, 5, 6A, 6B, 7F, 8, 9N, 9V, 10A, 11A, 12F, 14, 15A, 15B, 15C, 18C, 19A, 19F, 23A, 23B, 22F, 23F, 24F, 33F, and 35B.

3. (canceled)

4. (canceled)

5. (canceled)

6. (canceled)

7. (canceled)

8. The multivalent pneumococcal conjugate composition of claim 2, wherein the protein carrier comprises CRM₁₉₇ and/or tetanus toxoid.

9. The multivalent pneumococcal conjugate composition of claim 8, wherein at least two of the capsular polysaccharides are conjugated to tetanus toxoid and the remaining capsular polysaccharides are conjugated to CRM₁₉₇, wherein the at least two capsular polysaccharides that are conjugated to tetanus toxoid are selected from the group consisting of serotypes 1, 3, 5, 15B, and 22F.

10. The multivalent pneumococcal conjugate composition of claim 2, wherein the capsular polysaccharides from serotypes 1 and 5 are conjugated to the tetanus toxoid, and the capsular polysaccharides from serotypes 3, 4, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B are conjugated to CRM₁₉₇.

11. The multivalent pneumococcal conjugate composition of claim 2, wherein the capsular polysaccharides from serotypes 1 and 3 are conjugated to the tetanus toxoid, and the capsular polysaccharides from serotypes 4, 5, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B are conjugated to CRM₁₉₇.

12. The multivalent pneumococcal conjugate composition of claim 2, wherein the capsular polysaccharides from serotypes 3 and 5 are conjugated to the tetanus toxoid, and the capsular polysaccharides from serotypes 1, 4, 6A, 6B, 7F, 8, 9N, 9V, 10A, 11A, 12F, 14, 15A, 15B, 15C, 18C, 19A, 19F, 22F, 23A, 23B, 23F, 24F, 33F, and 35B are conjugated to CRM₁₉₇.

13. The multivalent pneumococcal conjugate composition of claim 2, wherein the capsular polysaccharides from serotypes 1, 5, 15B, and 22F are conjugated to the tetanus toxoid, and the capsular polysaccharides from serotypes 3, 4, 6A, 6B, 7F, 8, 9N, 9V, 10A, 11A, 12F, 14, 15A, 15C, 18C, 19A, 19F, 23A, 23B, 23F, 24F, 33F, and 35B are conjugated to CRM₁₉₇.

14. The multivalent pneumococcal conjugate composition of claim 2, wherein the capsular polysaccharides from serotypes 1, 3, 15B, and 22F are conjugated to the tetanus toxoid, and the capsular polysaccharides from serotypes 4, 5, 6A, 6B, 7F, 8, 9N, 9V, 10A, 11A, 12F, 14, 15A, 15C, 18C, 19A, 19F, 23A, 23B, 23F, 24F, 33F, and 35B are conjugated to CRM₁₉₇.

15. The multivalent pneumococcal conjugate composition of claim 2, wherein the capsular polysaccharides from serotypes 3, 5, 15B, and 22F are conjugated to the tetanus toxoid, and the capsular polysaccharides from serotypes 1, 4, 6A, 6B, 7F, 8, 9N, 9V, 10A, 11A, 12F, 14, 15A, 15C, 18C, 19A, 19F, 23A, 23B, 23F, 24F, 33F, and 35B are conjugated to CRM₁₉₇.

16. The multivalent pneumococcal conjugate composition of claim 9, further comprising an adjuvant.

17. The multivalent pneumococcal conjugate composition of claim 16, wherein the adjuvant is an aluminum-based adjuvant.

18. The multivalent pneumococcal conjugate composition of claim 17, wherein the adjuvant is selected from the group consisting of aluminum phosphate, aluminum sulfate, and aluminum hydroxide.

19. The multivalent pneumococcal conjugate composition of claim 18, wherein the adjuvant is aluminum phosphate.

20. (canceled)

21. A vaccine comprising the multivalent pneumococcal conjugate composition of claim 9 and a pharmaceutically acceptable excipient.

22. A method for prophylaxis of Streptococcus pneumoniae infection or disease in a subject, the method comprising administering a prophylactically effective amount of the multivalent pneumococcal conjugate composition of or the vaccine of claim 21 to the subject.

23. The method of claim 22, wherein the subject is a human who is at least 50 years old and the disease is pneumonia or invasive pneumococcal disease (IPD).

24. The method of claim 22, wherein the subject is a human who is at least 6 weeks old and the disease is pneumonia, invasive pneumococcal disease (IPD), or acute otitis media (AOM).

25. The method of claim 24, wherein the subject is 6 weeks to 5 years of age, 2 to 15 months of age, or 6 to 17 years of age.

26. The method of claim 22, wherein the subject is a human.

27. The method of claim 22, wherein the multivalent pneumococcal conjugate composition or the vaccine is administered by intramuscular injection.

28. The method of claim 22, wherein the multivalent pneumococcal conjugate composition or the vaccine is administered as part of an immunization series.

29. An immunogenic composition comprising at least one polysaccharide-protein conjugate, wherein the polysaccharide in the at least one polysaccharide-protein conjugate is a capsular polysaccharide from Streptococcus pneumoniae serotype 15A, serotype 15C, serotype 23A, serotype 23B, serotype 24F, or serotype 35B.

30. A method of making a capsular polysaccharide from Streptococcus pneumoniae serotype 15A, serotype 15C, serotype 23A, serotype 23B, serotype 24F, or serotype 35B as described herein.

31. (canceled)

32. (canceled)

33. (canceled)

34. (canceled)

36. (canceled)
